(19) United States
(12) Patent Application Publication
(10) Pub. No.: US 2026/0115209 A1
(43) Pub. Date: Apr. 30, 2026

(54) COMBINATION THERAPY INCLUDING COX-2 INHIBITOR FOR THE TREATMENT OF CANCER

(71) Applicant: THE REGENTS OF THE UNIVERSITY OF CALIFORNIA, Oakland, CA (US)

(72) Inventor: Jean-Philippe COPPE, San Francisco, CA (US)

(21) Appl. No.: 19/101,992

(22) PCT Filed: Aug. 7, 2023
(86) PCT No.: PCT/US2023/071796

§ 371 Date: Feb. 7, 2025

Related U.S. Application Data
(60) Provisional application No. 63/396,165, filed on Aug. 8, 2022.

(51) Int. Cl. A61K 31/635; A61K 31/437; A61K 31/506; A61K 31/517; A61K 31/519; A61K 31/5377; A61K 39/395; A61P 35/00

(52) U.S. Cl. CPC A61K 31/635; A61K 31/437; A61K 31/506; A61K 31/517; A61K 31/519; A61K 31/5377; A61K 39/3955; A61P 35/00

(57) ABSTRACT

Provided herein, inter alia, are compositions and methods comprising combination therapies including a BRAE axis inhibitor and a COX-2 inhibitor for the treatment of cancer. In embodiments, the combination therapies further include an EGER inhibitor. The combination therapies provided herein are particularly effective for treating BRAE intrinsically resistant cancers.

### CROSS-REFERENCES TO RELATED APPLICATIONS

This application claims the benefit of U.S. Provisional Application No. 63/396,165, filed Aug. 8, 2022, which is incorporated herein by reference in its entirety and for all purposes.

### STATEMENT AS TO RIGHTS TO INVENTIONS MADE UNDER FEDERALLY SPONSORED RESEARCH AND DEVELOPMENT

This invention was made with government support under grant RO1 CA229447 awarded by The National Institutes of Health. The government has certain rights in the invention.

### REFERENCE TO AN ELECTRONIC SEQUENCE LISTING

The contents of the electronic sequence listing (048536-740001WO_Sequence_Listing_ST26.xml; Size: 27,995 bytes; and Date of Creation: Aug. 4, 2023) is hereby incorporated by reference in its entirety.

### BACKGROUND

Presence of a BRAF^{V600E} kinase mutation predicts the worst prognosis form of metastatic colorectal cancer (mCRC). About 8% of mCRCs harbor a BRAF^{V600E} mutation. Because mCRC is the second leading cause of cancer death, it is estimated that more patients die of BRAF^{V600E} mCRC than melanoma each year. Unlike melanoma, BRAF^{V600E} mCRC does not respond to BRAF inhibitor monotherapy, and it responds only poorly to conventional chemotherapy¹⁻³. Response rates have increased by combining BRAF inhibitors with MEK inhibitors and/or an anti-EGFR antibody, however the majority of mCRC tumors still fail to regress and durability of disease control remains a challenge. Clinical outcomes have been remarkably similar with combinations of BRAF inhibitors (vemurafenib, dabrafenib, or encorafenib) and/or MEK inhibitors (trametinib or binimetinib) and/or anti-EGFR antibodies (cetuximab or panitumumab): with a median confirmed response rate of ˜20%, progression free survival of ˜4 months, and overall survival of ˜9 months⁴⁻⁶. In April 2020, the Federal Drug Administration (FDA) granted approval to the encorafenib plus cetuximab doublet for the treatment of patients with BRAF^{V600E} mCRC, based on comparable outcomes with this doublet vs. triplet combinations evaluated in clinical trials.

The objective of targeting BRAF±MEK±EGFR is to reinforce the inhibition of the main oncogenic driver pathway (BRAF-MEK) while jointly shutting down the activation of a drug resistance mechanism (EGFR)^{5,6}. However, the observed ceiling effect with this approach suggests that other prevalent mechanisms (i.e. dependencies) must cooperate to circumvent therapeutic effectiveness.

Disclosed herein, inter alia, are solutions to these and other problems in the art.

### BRIEF SUMMARY OF THE INVENTION

Provided herein, inter alia, are compositions and methods for treating cancer. The compositions and methods provided herein include combination therapies including a BRAF axis inhibitor and a COX-2 inhibitor. In embodiments, the compositions and methods provided herein further include an EGFR inhibitor.

In an aspect is provided a method of treating a cancer in a subject, including administering to the subject a combined effective amount of a serine/threonine-protein kinase B-Raf (BRAF) axis inhibitor and a cyclooxygenase-2 (COX-2) inhibitor.

In another aspect is provided a method of treating a serine/threonine-protein kinase B-Raf (BRAF) resistant cancer in a subject, including administering to the subject a combined effective amount of BRAF axis inhibitor and a cyclooxygenase-2 (COX-2) inhibitor.

In an aspect is provided a method of treating a serine/threonine-protein kinase B-Raf (BRAF) intrinsically resistant cancer in a subject, including administering to the subject a combined effective amount of a BRAF axis inhibitor and a cyclooxygenase-2 (COX-2) inhibitor.

In an aspect is provided a method of treating cancer in a subject, including administering to the subject a combined effective amount of a BRAF axis inhibitor, an epidermal growth factor receptor (EGFR) inhibitor, and a cyclooxygenase-2 (COX-2) inhibitor.

In an aspect is provided a method of treating a serine/threonine-protein kinase B-Raf (BRAF) resistant cancer in a subject, including administering to the subject a combined effective amount of a BRAF axis inhibitor, an epidermal growth factor receptor (EGFR) inhibitor, and a cyclooxygenase-2 (COX-2) inhibitor.

In an aspect is provided a method of treating a serine/threonine-protein kinase B-Raf (BRAF) intrinsically resistant cancer in a subject, including administering to the subject a combined effective amount of a BRAF axis inhibitor, an epidermal growth factor receptor (EGFR) inhibitor, and a cyclooxygenase-2 (COX-2) inhibitor.

In an aspect is provided a pharmaceutical kit including: (i) a serine/threonine-protein kinase B-Raf (BRAF) axis inhibitor; and (b) a and a cyclooxygenase-2 (COX-2) inhibitor.

In another aspect is provided a pharmaceutical kit including: (i) a serine/threonine-protein kinase B-Raf (BRAF) axis inhibitor; (ii) an epidermal growth factor receptor (EGFR) inhibitor, and (iii) a and a cyclooxygenase-2 (COX-2) inhibitor.

### BRIEF DESCRIPTION OF THE DRAWINGS

FIGS. 1A-1G. SRC is activated following BRAF/MEK/EGFR inhibition in BRAF^{V600}E CRC. FIGS. 1A-1B, Unsupervised hierarchical clustering of the phospho-catalytic activity signatures of WiDr cells treated with vemurafenib (VEM; n=13)±gefitinib (GEF; n=5) or cetuximab (CET; n=5) as compared to their untreated control counterparts (n=23). FIG. 1A, ATP consumption in cell extracts using 228 peptide sensors. FIG. 1B, Kinase signatures deconvoluted from peptide phosphorylation profiles in (FIG. 1A). Bar graphs next to the heatmaps display the p-values (p-val) for each of the peptides (FIG. 1A) or kinases (FIG. 1B) comparing all treated samples to controls. FIG. 1C, Volcano plot of data in (FIG. 1B) displays change in kinase activity versus p-values for each treatment arm. FIG. 1D, Bar graphs representing the shift in activity of SRC, SFK, EGFR and HER family kinases when cells are treated with VEM alone or in combination with GEF or CET. Kinase activity is compared to the untreated control cells and data displayed as the average±standard error. FIG. 1E, Representative IHC images showing active SFK (phosphotyrosine-419 epitope in the SRC activation site) staining intensity following treatment of a BRAF^{V600E} CRC PDX model with vehicle control, dabrafenib (DAB) and/or trametinib (TRA) for 3 or 21 days. The bottom panel highlights differences in bin intensities resulting from automated image analysis. FIG. 1F, Quantification of IHC staining intensity for total and activated SFK in two PDX models treated for 3 or 21 days with DAB±TRA vs. vehicle control. Intensity levels (e.g. 4-bin intensity scale) decrease from top to bottom for each bar shown. FIG. 1G, Proposed parallel mechanism of SRC activation in response to BRAF/MEK/EGFR therapies in BRAF^{V600E} CRC. BRAF*: BRAF^{V600E}.

FIGS. 2A-2E. SRC kinase inhibition sensitizes BRAF^{V600E} CRC cell lines to vemurafenib. FIG. 2A, Cell viability assays evaluating WiDr cells treated with vemurafenib (VEM) plus a panel of kinase inhibitors selected based on results in (FIG. 1B). The size of the bubble indicates the magnitude of the change in kinase activity induced by VEM treatment (FIG. 1), with gray scale signifying increased (light gray) or decreased (dark gray) activity; y-axis: log 2 scale; rs: Spearman's rho correlation, p: p-value for 2-tailed t-test. FIG. 2B, Shift in VEM sensitivity, measured via cell viability assays (left panel) and calculation of the combination index (right panel) upon treatment of BRAF^{V600E} CRC or melanoma (MEL) cell lines with VEM together with a SRC inhibitor: dasatinib (DAS), saracatinib (SAR) or bosutinib (BOS), or an EGFR inhibitor: gefitinib (GEF), for three days. CI scores are averaged from individual experimental CIs calculated at 1×GI50, 2×GI50, 0.5×GI50 concentrations of each drug (n≥2). FIG. 2C, Colony formation assays where BRAF^{V600E} CRC or melanoma (Mel888) cells were treated with an increasing concentration of VEM alone (control, CON) or with a fixed dose of DAS. FIG. 2D, Western blot showing knockdown of SRC in BRAF^{V600E} CRC cell lines stably transfected with a control shRNA (shCON) or two different SRC-targeting shRNAs (shSRC). HSP90 serves as loading control. FIG. 2E, Bar graphs representing fold-change (log 2 scale)±standard error for change in sensitivity to VEM with knockdown of SRC in 3-day cell viability assays. Top panel: combination index, Bliss model score. FIG. 2F, Colony formation in BRAF^{V600E} CRC cells treated with an increasing concentration of VEM with or without SRC knockdown.

FIGS. 3A-3I. Coordinated targeting of SRC with BRAF+EGFR increases efficacy in BRAF^{V600E} CRC cell lines and xenografts. FIG. 3A, BRAF^{V600E} CRC cell lines treated with vemurafenib (VEM)±gefitinib (GEF) were lysed and immunoblotted with the indicated antibodies. SFK activation is reflected by increased phosphorylation of the SRC activation site, tyrosine 419 (pY419), and non-phosphorylation of the inhibitory site, tyrosine 530 (non-pY530). Active SRC can be deactivated by re-phosphorylation of Y530 by C-terminal SRC kinase (CSK). HSP90 serves as loading control. FIG. 3B, Shift in VEM sensitivity measured via cell viability assays (left panel) and calculation of the combination index (right panel) upon treatment of BRAF^{V600E} CRC or melanoma (MEL) cell lines with VEM+GEF±a SRC inhibitor, dasatinib (DAS), for three days. FIG. 3C, Colony formation assays where BRAF^{V600E} CRC cells were treated with an increasing concentration of VEM alone (control, CON) or with a fixed dose of GEF±DAS. FIG. 3D, Treatment of cell line-derived xenograft mouse models with a VEM progenitor, PLX4720 (PLX); DAS; saracatinib (SAR); and/or GEF for 21 days. Plotted is the percent change in tumor volume relative to baseline (day 1). Data is displayed as the average for all mice in a specified treatment group±standard error. FIG. 3E, Treatment of patient-derived xenograft models with VEM±GEF±DAS for 21 days, plotted as in (FIG. 3D). FIGS. 3F-3G, Generalized Linear Model (GLM) to test the association of change in tumor volume between treatment arms and vehicle over time. Effect size is measured as the GLM standard coefficient. GLM was applied to each tumor model separately or combined. Results for cell line xenografts are shown in FIG. 3F and results for the PDXs are shown in FIG. 3G. GLM p-values corrected for false discovery rate (FDR) are shown in FIG. 3G. FIGS. 3H-3I, Comparison of effect size and FDR-corrected p-values of treatment arms with (FIG. 3H) and without (FIG. 3I) a SRC inhibitor.

FIGS. 4A-4D. SRC regulates the phosphorylation of beta-catenin. FIG. 4A, Western blot of BRAF^{V600E} CRC cell lines treated with VEM±GEF or DAS. The tyrosine 654 (Y654) of beta-catenin (CTNNB1) is a reported phospho-target site of SRC kinases 8. ERK1/2 phospho-T202/Y204 serves as a control for the effect of BRAF-inhibition (with VEM). SFK phospho-Y419 serves as a control for the effect of SFK-inhibition (with DAS). FIG. 4B, Expression levels of beta-catenin target genes (MYC, AXTN2, ASCL2, S100A6, LEF1, NOTCH2, SP5) measured using quantitative real time (qRT) PCR in BRAF^{V600E} CRC cell lines treated with VEM±GEF or DAS. Expression profiles are shown as fold change against the mean mRNA expression level in VEM, VEM+GEF, VEM+DAS. Percentages indicate the proportion of measurements across 8 cell lines where the expression of the indicated gene (top) was lower with VEM+DAS than with VEM+GEF or VEM alone. The right-most columns indicate p-values (student t-test) comparing gene expression in VEM+DAS versus VEM alone. n.a.: not available due to expression levels that were too low. FIG. 4C, The expression profiles displayed in (FIG. 4B) were averaged across cell lines. FIG. 4D, Proposed mechanism regulated by SRC and that drives resistance to BRAF/MEK/EGFR therapies in BRAF^{V600E} CRC.

FIGS. 5A-5J. COX2/PGE2 upregulation mediates SRC activation in BRAF^{V600E} CRC cell lines and PDXs. FIG. 5A, PGE2 secreted levels were measured by ELISA in the conditioned media of BRAF^{V600E} CRC cell lines treated with vemurafenib (VEM)±gefitinib (GEF). Data for untreated, VEM treated cells, and VEM+GEF treated cells are shown from left to right for each cell line indicated. Data is displayed as the average PGE2 secretion in pg/mL per 100,000 cells±standard deviation. FIG. 5B, BRAF^{V600E} CRC cell lines were treated with exogenous PGE2. Cell lysates were assayed by western blot as indicated. Y419 phosphorylation and lack of phosphorylation of Y530 (non-pY530) are used as readouts of SFK activation. HSP90 serves as loading control. FIG. 5C, Bar graphs representing fold-change (log 2 scale)±standard error for change in sensitivity to VEM upon further treatment with PGE2 or untreated control (CON) in 3-day cell viability assays. Top panel: combination index, Bliss model score. FIG. 5D, Three BRAF^{V600E} CRC cell lines engineered with a doxycycline-inducible constitutively active GNAS construct, iGNAS^{R201C}, were treated with doxycycline. Cell lysates were assayed by western blot as indicated. FIG. 5E, Bar graphs representing fold-change (log 2 scale)±standard error for change in sensitivity to VEM or VEM+GEF after iGNAS^{R201C} induction in 3-day cell viability assays. Top panel: combination index, as in (FIG. 5C). FIG. 5F, GNAS was knocked out in BRAF^{V600E} CRC cells using CRISPR (GNAS-KO). GNAS-KO was validated by western blot (top panel). GNAS-KO cells were treated with VEM and cell lysates were assayed by western blot with the indicated antibodies (bottom panel). FIG. 5G, Bar graphs representing fold-change (log 2 scale)±standard error for change in sensitivity to VEM or VEM+GEF with GNAS-KO in 3-day cell viability assays. Top panel: combination index, as in (FIG. 5C). FIG. 5H, Representative immunohistochemistry (IHC) images showing COX2 staining intensity following treatment of a BRAF^{V600E} CRC PDX model with vehicle control, dabrafenib (DAB) and/or trametinib (TRA) for 3 or 21 days. The bottom panel highlights differences in bin intensities resulting from automated image analysis. FIG. 5I, Quantification of COX2 staining intensity by IHC for two PDX models treated for 3 or 21 days with DAB±TRA vs. vehicle control. The intensity levels (e.g. 4-bin intensity scale) decrease from top to bottom for each bar. FIG. 5J, Proposed mechanism of COX2/PEG2 mediated SRC-driven resistance to BRAF/MEK/EGFR therapies in BRAF^{V600E} CRC.

FIGS. 6A-6G. Coordinated targeting of COX2 with BRAF/MEK/EGFR improves efficacy in BRAF^{V600E} CRC cell lines and PDXs. FIG. 6A, Shift in vemurafenib (VEM) sensitivity measured via cell viability assays (left panel) and calculation of the combination index (right panel) upon treatment of BRAF^{V600E} CRC or melanoma (MEL) cell lines with VEM together with a COX2 inhibitor: celecoxib (CEL) or valdecoxib (VAL), for three days. CI averaged from experimentally measured CIs at 1×GI50, 2×GI50, 0.5×GI50 concentrations of each drug (n>2). FIG. 6B, Treatment of BRAF^{V600E} CRC or MEL cell lines with up to four inhibitors: TRA, GEF and CEL at concentrations that result in at most 10% of maximal inhibition of cell proliferation (GI10) when dosed individually. Cell growth inhibition across treatments permutations, normalized to VEM monotherapy (left panel), was used to calculate the combination index relative to all other treatment arms and subjected to unsupervised hierarchical clustering comparing cell lines and treatment arms (right panel). FIG. 6C, Mouse weight as a surrogate for toxicity following treatment of BRAF^{V600E} CRC PDXs (23 mice per treatment arm) with vehicle control, dabrafenib (DAB), trametinib (TRA), celecoxib (CEL) and/or PAN (panitumumab). Data is displayed as the average weight in grams±standard deviation. FIG. 6D, Tumor growth inhibition in BRAF^{V600E} CRC PDX models following treatment with DAB+TRA±CEL±PAN or vehicle (control). Waterfall plots show the relative change in tumor volume: each bar represents one tumor; and the height of the bar compares the final volume at day 21(d21) to the starting volume at day 1. Volume changes are capped at 2-fold of the starting volume (i.e. 200%). Average final tumor volumes per treatment group are indicated underneath the graph (black font). T-test p-values are indicated when p<0.05. FIG. 6E, Semi-supervised hierarchical clustering of the percentages of regressing tumors per treatment arm are indicated, comparing day 21 vs. day 1 and day 21 vs. day 10. FIG. 6F, Generalized Linear Model (GLM) to test the association of change in tumor volume between treatment arms and vehicle over time. GLM was applied to each PDX model and all PDX combined. Left panel, effect size measured as the GLM standard coefficient; semi-unsupervised hierarchical clustering further compares the efficacy of the treatment arms. Right panel, ranking by GLM p-values corrected for false discovery rate (FDR). FIG. 6G, Comparison of effect size and FDR-corrected p-values of treatment arms with and without the addition of celecoxib.

FIGS. 7A-7F. Coordinated targeting of COX2 with BRAF+EGFR improves long term efficacy in BRAF^{V600E} CRC PDXs. FIG. 7A-7B, Tumor growth profiles in BRAF^{V600E} CRC PDX models #1 (FIG. 7A) and #2 (FIG. 7B) treated with ENC±PAN±CEL or vehicle (control). Data markers for vehicle (control) are triangle; ENC are diamonds; ENC+PAN are squares; and ENC+PAN+CEL are circles. Changes in tumor volumes relative to starting volume at day 1 (average±standard error) are plotted over time. Student t-test p-values across all time points comparing treatment arms are shown as a grey scale underneath each graph. n.s. not significant; x: not available. FIG. 7C, GLM to test the association of change in tumor volume over time, either between treatment arms and vehicle (left panel) or between combination therapy and ENC alone (right panel). GLM was applied to each individual PDX model and to both PDXs combined. The top section shows effect size measured as the GLM standard coefficient comparing the efficacy of the treatment arms. The bottom section shows GLM FDR-corrected p-values. FIG. 7D, Comparison of effect size and FDR-corrected p-values of treatment arms with and without celecoxib, using vehicle or ENC treatment as the baseline (respectively left and right). FIG. 7E, Mouse weight as a surrogate for treatment toxicity. Data is displayed as the average weight in grams±standard deviation. FIG. 7F, Schematic summary of the states of signaling pathways depending on treatment: (i) untreated tumors, with BRAF-MEK-ERK as the main driver of progression, and baseline activity of the EGFR and COX2/SRC signaling pathways; (ii-iv) tumors treated with drugs (listed on top) inhibiting the activity of the three distinct signaling axes: BRAF-MEK, EGFR and COX2-SRC-beta Catenin; (iv) triple treatment collectively blocks the cooperative dependencies that drive resistance and progression.

FIGS. 8A-8D. SRC is activated consequent to BRAF/MEK/EGFR inhibition in BRAF^{V600E} CRC specifically. FIG. 8A, BRAF^{V600E} CRC cell lines were treated with vemurafenib (VEM) for 7 h. Vemurafenib was used at 1.5 uM in WiDr, 1.75 uM in HT29 and 0.15 uM in LIM2405 and SNUC5 cells. Cell lysates were assayed by western blot with the indicated antibodies. Upper panels: SFK activation is reflected by increased phosphorylation of the SRC activation site, Y419 (pY419). HSP90 is used as loading control. Bottom panel: reduction in ERK 1/2 phosphorylation as control of BRAF inhibition. FIG. 8B, Representative IHC images showing total SRC staining intensity following treatment of a BRAF^{V600E} CRC PDX model with vehicle control, dabrafenib (DAB) and/or trametinib (TRA) for 3 or 21 days. The bottom panel highlights differences in bin intensities resulting from automated image analysis. FIG. 8C, Quantification of IHC staining intensity for total and activated SFK in two PDX models treated for 3 or 21 days with DAB±TRA vs. vehicle control. FIG. 8D, SRC staining score by IHC in untreated patient CRC tumor specimens with or without a BRAF^{V600E} mutation, from primary (prim.) or metastatic (met.) sites.

FIGS. 9A-9C. SRC kinase activity is inversely correlated with sensitivity to vemurafenib in BRAF^{V600E} CRC cell lines. FIG. 9A, Shift in vemurafenib (VEM) sensitivity, measured via cell viability assays upon treatment of BRAF^{V600E} or KRAS mutated or MAP3K8 amplified CRC, or melanoma (MEL) cell lines with VEM together with: a SRC inhibitor, dasatinib (DAS), saracatinib (SAR) or bosutinib (BOS), or an EGFR inhibitor, gefitinib (GEF), for three days. Data for VEM+DAS, VEM+SAR, VEM+BOS, and VEM+GEF are shown from left to right for each cell line indicated. Calculation of the combination index (CI score) was also completed. FIG. 9B, BRAF^{V600E} CRC cell lines were transfected with a siRNA against CSK, a negative regulator of SFKs, and siRNA Control. Cell lysates were assayed by western blot with the indicated antibodies, showing the effect of CSK depletion on SFK activation. FIG. 9C, Bar graphs representing fold-change (log scale)±standard error for change in sensitivity to VEM with knockdown of CSK in 3-day cell viability assays. Top panel: combination index, Bliss model score.

FIGS. 10A-10E. Coordinated targeting of SRC with BRAF±EGFR improves efficacy in BRAF^{V600E} CRC cell lines and xenografts without increasing toxicity. FIG. 10A, Shift in vemurafenib (VEM) sensitivity, measured via cell viability assays upon treatment of BRAF^{V600E} CRC or melanoma (MEL) cell lines with VEM+gefitinib (GEF)±dasatinib (DAS) and VEM+DAS±GEF, for three days. The addition of DAS to VEM+GEF increases sensitivity to VEM to a greater extent than the addition of GEF to VEM+DAS, highlighting the contribution of SFK and supporting that SFK activation upon VEM treatment is EGFR-independent. Data for VEM+GEF, VEM+GEF+DAS, VEM+DAS, and VEM+DAS+GEF are shown from left to right for each cell line indicated. Calculation of the combination index (CI score) was also completed. FIGS. 10B-10C, Mouse weight as a surrogate for toxicity following treatment of BRAF^{V600E} CRC cell line- (FIG. 10B) or patent-derived xenografts (FIG. 10C) with vehicle control or the inhibitors listed (PLX: PLX4720; SAR: saracatinib). Data is displayed as the average weight in grams±standard deviation. FIG. 10D, Tumor growth inhibition in BRAF^{V600E} CRC PDX models following treatment with VEM±GEF±DAS or vehicle (control). Waterfall plots show the relative change in tumor volume: each bar represents one tumor; and the height of the bar compares the final volume at day 21 to the starting volume at day 1. Volume changes are capped at 5-fold of the starting volume (i.e. 500%). Average final tumor volumes per treatment group are indicated underneath the graph (black font). T-test p-values are indicated when p<0.05. Percentages of regressing tumors per group are indicated underneath the graph (gray font). FIG. 10E, The GLM p-values corrected for false discovery rate (FDR) corresponding to the main in (FIG. 3F) are shown.

FIGS. 11A-11C. Mechanisms underlying the synergistic effects of co-targeting SRC and the BRAF±EGFR pathways. FIG. 11A, Western blots to detect phospho-T202/Y204 ERK1/2 and total ERK1/2 in BRAF^{V600E} CRC cell lines treated with vemurafenib (VEM)±gefitinib (GEF) or dasatinib (DAS) collected after 8 h, 24 h, 48 h or 72 h. HSP90 is used as a loading control. FIG. 11B, Quantification of western blots shown in panel (FIG. 11A). The bar plot (averages and standard deviations per treatment condition across cell lines) was overlaid with a dot plot displaying individual measurements per cell line and condition. Data are normalized to p-ERK levels after 8 h treatment with VEM alone. See table below for detailed values. FIG. 11C, Western blots to detect total and phospho-Y654 beta-catenin (CTNNB1) in BRAF^{V600E} CRC cell lines treated with VEM, or GEF, or DAS, or combinations of VEM+GEF, or VEM+DAS, or VEM+GEF+DAS. The detection of phospho-Y419 and total SFK serves as a control for the effect of SFK-inhibition (with DAS).

FIGS. 12A-12D. PGE2/GNAS signaling in BRAF^{V600E} CRC cell lines and tumors FIG. 12A, Western blots to detect phospho-Y654 of beta-catenin (CTNNB1) in BRAF^{V600E} CRC cell lines treated with exogenous PGE2. FIGS. 12B-12C, Western blots to detect phospho-T202/Y204 ERK1/2, total ERK1/2, phospho-S217/S221 MEK1/2, total MEK1/2 in BRAF^{V600E} CRC cell lines modified for GNAS expression, and treated with vemurafenib (VEM). FIG. 12B, BRAF^{V600E} CRC cell lines engineered with a doxycycline-inducible constitutively active GNAS construct, iGNAS^{R201C} with or without doxycycline treatment (+ or − at the bottom of each panel). FIG. 12C, BRAF^{V600E} CRC cell lines knocked out for GNAS using CRISPR (GNAS-KO or CRIPSR control; indicated as + or − at the bottom of each panel). FIG. 12D, COX2 staining score by IHC in untreated patient CRC tumor specimens with or without a BRAF^{V600E} mutation, from primary (prim.) or metastatic (met.) sites.

### DETAILED DESCRIPTION

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. For example, the nucleic acid provided herein may be part of a vector. For example, the nucleic acid provided herein may be part of a lentiviral vector, which may be transduced into a cell. Polynucleotides useful in the methods of the disclosure may comprise natural nucleic acid sequences and variants thereof, artificial nucleic acid sequences, or a combination of such sequences.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 75%, 85%, 90%, 91%, 92%, 93%, 94%, 95%, 96%, 97%, 98%, 99%, or higher identity over a specified region).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

The term “amino acid side chain” refers to the functional substituent contained on amino acids. For example, an amino acid side chain may be the side chain of a naturally occurring amino acid. Naturally occurring amino acids are those encoded by the genetic code (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine), as well as those amino acids that are later modified, e.g., hydroxyproline, γ-carboxyglutamate, and O-phosphoserine. In embodiments, the amino acid side chain may be a non-natural amino acid side chain. In embodiments, the amino acid side chain is H,

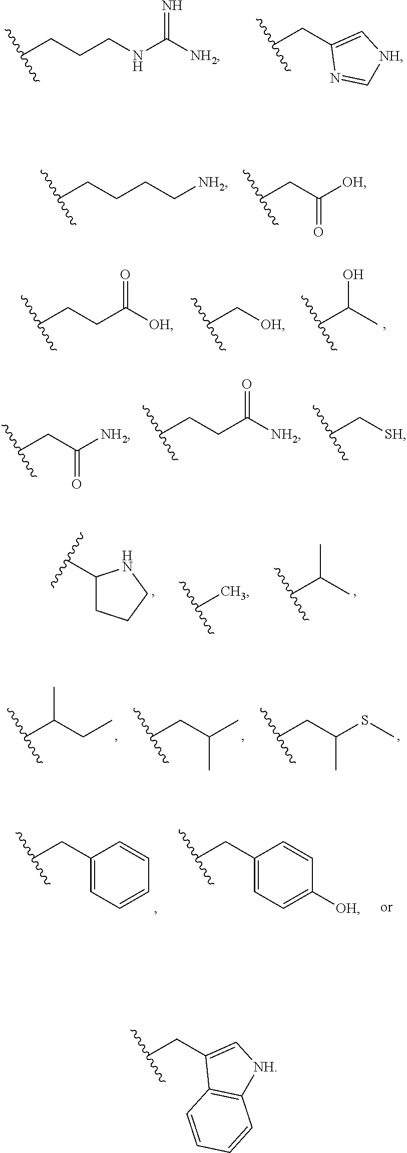

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may In embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers.

A “fusion protein” refers to a chimeric protein encoding two or more separate protein sequences that are recombinantly expressed as a single moiety. Because the different proteins in fusion proteins may affect the functionality of other proteins under certain circumstances, peptide linkers may be used between different proteins within the same fusion protein. These peptide linkers may have a flexible structure and separate the proteins within the fusion protein so that each protein in the fusion proteins substantially retains its function. Peptide linkers are known in the art and described, for example, in Chen et al, Adv Drug Deliv Rev, 65(10); 1357-1369 (2013).

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. One skilled in the art will immediately recognize the identity and location of residues corresponding to a specific position in a protein in other proteins with different numbering systems. For example, by performing a simple sequence alignment with a protein the identity and location of residues corresponding to specific positions of the protein are identified in other protein sequences aligning to the protein. For example, a selected residue in a selected protein corresponds to glutamic acid at position 138 when the selected residue occupies the same essential spatial or other structural relationship as a glutamic acid at position 138. In some embodiments, where a selected protein is aligned for maximum homology with a protein, the position in the aligned selected protein aligning with glutamic acid 138 is the to correspond to glutamic acid 138. Instead of a primary sequence alignment, a three dimensional structural alignment can also be used, e.g., where the structure of the selected protein is aligned for maximum correspondence with the glutamic acid at position 138, and the overall structures compared. In this case, an amino acid that occupies the same essential position as glutamic acid 138 in the structural model is the to correspond to the glutamic acid 138 residue.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
(see, e.g., Creighton, Proteins (1984)).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 75%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. The preferred algorithms can account for gaps and the like. Preferably, identity exists over a region that is at least about 25 amino acids or nucleotides in length, or more preferably over a region that is 50-100 amino acids or nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

For specific proteins described herein, the named protein includes any of the protein's naturally occurring forms, variants or homologs that maintain activity of the protein (e.g., within at least 50%, 75%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference, homolog or functional fragment thereof.

The term “BRAF protein” or “BRAF” as used herein includes any of the recombinant or naturally-occurring forms of Serine/threonine-protein kinase B-raf (BRAF) also known as, Proto-oncogene B-Raf, v-Raf murine sarcoma viral oncogene homolog B1, or variants or homologs thereof that maintain BRAF activity (e.g. within at least 50%, 80%, 90%, 95%, 96%, 97%, 98%, 99% or 100% activity compared to BRAF). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring BRAF protein. In embodiments, the BRAF protein is substantially identical to the protein identified by the UniProt reference number P³15056 or a variant or homolog having substantial identity thereto. In embodiments, the BRAF protein is substantially identical to the sequence of SEQ ID NO:23 or a variant or homolog having substantial identity thereto.

In embodiments, the BRAF protein includes a valine to glutamic acid mutation at a position corresponding to position 600 relative to UniProt reference number P15056. In embodiments, the BRAF protein includes a valine to lysine mutation at a position corresponding to position 600 relative to UniProt reference number P³15056. In embodiments, the BRAF protein includes a valine to aspartic acid mutation at a position corresponding to position 600 relative to UniProt reference number P15056. In embodiments, the BRAF protein includes a valine to arginine mutation at a position corresponding to position 600 relative to UniProt reference number P15056. In embodiments, the BRAF protein includes a lysine to glutamic acid mutation at position 601 relative to UniProt reference number P15056. In embodiments, the BRAF protein includes a lysine to asparagine mutation at a position corresponding to position 601 relative to UniProt reference number P15056. In embodiments, the BRAF protein includes a leucine to serine mutation at a position corresponding to position 597 relative to UniProt reference number P15056. In embodiments, the BRAF protein includes a leucine to arginine mutation at a position corresponding to position 597 relative to UniProt reference number P15056. In embodiments, the BRAF protein includes a leucine to glutamine mutation at a position corresponding to position 597 relative to UniProt reference number P15056.

In embodiments, the BRAF protein includes a valine to glutamic acid mutation at a position corresponding to position 600 relative to SEQ ID NO:23. In embodiments, the BRAF protein includes a valine to lysine mutation at a position corresponding to position 600 relative to SEQ ID NO:23. In embodiments, the BRAF protein includes a valine to aspartic acid mutation at a position corresponding to position 600 relative to SEQ ID NO:23. In embodiments, the BRAF protein includes a valine to arginine mutation at a position corresponding to position 600 relative to SEQ ID NO:23. In embodiments, the BRAF protein includes a lysine to glutamic acid mutation at position 601 relative to SEQ ID NO:23. In embodiments, the BRAF protein includes a lysine to asparagine mutation at a position corresponding to position 601 relative to SEQ ID NO:23. In embodiments, the BRAF protein includes a leucine to serine mutation at a position corresponding to position 597 relative to SEQ ID NO:23. In embodiments, the BRAF protein includes a leucine to arginine mutation at a position corresponding to position 597 relative to SEQ ID NO:23. In embodiments, the BRAF protein includes a leucine to glutamine mutation at a position corresponding to position 597 relative to SEQ ID NO:23.

In embodiments, BRAF is BRAF^{V600E}. In embodiments, BRAF is BRAF^{V600K}. In embodiments, BRAF is BRAF^{V600D}. In embodiments, BRAF is BRAF^{V600R}. In embodiments, BRAF is BRAF^{K6001E}. In embodiments, BRAF is BRAF^{K6001N}. In embodiments, BRAF is BRAF^{L597S}. In embodiments, BRAF is BRAF^{L597R}. In embodiments, BRAF is BRAF^{L597Q}.

The term “MEK protein” or “MEK” as used herein includes any of the recombinant or naturally-occurring forms of mitogen-activated protein kinase kinase (MEK) also known as dual specificity mitogen-activated protein kinase kinase 2, or variants or homologs thereof that maintain MEK activity (e.g. within at least 50%, 80%, 90%, 95%, 96%, 97%, 98%, 99% or 100% activity compared to MEK). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring MEK protein. In embodiments, the MEK protein is substantially identical to the protein identified by the UniProt reference number P36507 or a variant or homolog having substantial identity thereto.

The term “ERK protein” or “ERK” as used herein includes any of the recombinant or naturally-occurring forms of Mitogen-activated protein kinase 3 also known as Extracellular signal-regulated kinase 1, Microtubule-associated protein 2 kinase, MAP kinase 3, or variants or homologs thereof that maintain ERK activity (e.g. within at least 50%, 80%, 90%, 95%, 96%, 97%, 98%, 99% or 100% activity compared to ERK). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring ERK protein. In embodiments, the ERK protein is substantially identical to the protein identified by the UniProt reference number P27361 or a variant or homolog having substantial identity thereto.

The term “COX-2 protein” or “COX-2” as used herein includes any of the recombinant or naturally-occurring forms of Prostaglandin G/H synthase 2 (COX-2) also known as Cyclooxygenase-2, PHS II, or variants or homologs thereof that maintain COX-2 activity (e.g. within at least 50%, 80%, 90%, 95%, 96%, 97%, 98%, 99% or 100% activity compared to COX-2). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring COX-2 protein. In embodiments, the COX-2 protein is substantially identical to the protein identified by the UniProt reference number P35354 or a variant or homolog having substantial identity thereto.

The term “EGFR protein” or “EGFR” as used herein includes any of the recombinant or naturally-occurring forms of epidermal growth factor receptor (EGFR) also known as ErbB-1 or HER1 in humans, or variants or homologs thereof that maintain EGFR activity (e.g. within at least 50%, 80%, 90%, 95%, 96%, 97%, 98%, 99% or 100% activity compared to EGFR). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring EGFR protein. In embodiments, the EGFR protein is substantially identical to the protein identified by the UniProt reference number P00533 or a variant or homolog having substantial identity thereto.

The term “SRC protein” or “SRC” as used herein includes any of the recombinant or naturally-occurring forms of Proto-oncogene tyrosine-protein kinase Src (SRC) also known as Proto-oncogene c-Src, or variants or homologs thereof that maintain SRC activity (e.g. within at least 50%, 80%, 90%, 95%, 96%, 97%, 98%, 99% or 100% activity compared to SRC). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring SRC protein. In embodiments, the SRC protein is substantially identical to the protein identified by the UniProt reference number P12931 or a variant or homolog having substantial identity thereto.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be, for example, a nucleic acid as provided herein and a cell. In embodiments contacting includes, for example, allowing a cancer therapeutic (e.g. BRAF axis inhibitor, COX-2 inhibitor, etc.) as described herein to interact with a cancer cell. In embodiments contacting includes, for example, allowing a combination therapy including a plurality of cancer therapeutics as described herein to interact with a cancer cell.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization. In embodiments, the cell is a cancer cell. In embodiments, the cancer is a BRAF intrinsically resistant cancer.

The term “inhibition”, “inhibit”, “inhibiting” and the like in reference to a protein-inhibitor interaction means negatively affecting (e.g. decreasing) the activity or function of the protein (e.g. BRAF, COX-2, EGFR, etc.) relative to the activity or function of the protein in the absence of the inhibitor. In aspects inhibition means negatively affecting (e.g. decreasing) the concentration or levels of the protein relative to the concentration or level of the protein in the absence of the inhibitor. In aspects inhibition refers to reduction of a disease or symptoms of disease. In aspects, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity (e.g. kinase activity (e.g. phosphorylation), etc.) or the amount of a protein. In aspects, inhibition refers to a reduction of activity of a target protein resulting from a direct interaction (e.g. an inhibitor binds to the target protein). In aspects, inhibition refers to a reduction of activity of a target protein from an indirect interaction (e.g. an inhibitor binds to a protein that activates the target protein, thereby preventing target protein activation).

The terms “inhibitor,” “repressor” or “antagonist” or “downregulator” interchangeably refer to a substance capable of detectably decreasing the expression or activity of a given gene or protein. The inhibitor can decrease expression or activity of the protein 10%, 20%, 30%, 40%, 50%, 60%, 70%, 75%, 90% or more in comparison to a control in the absence of the protein. In certain instances, expression or activity of the protein is 1.5-fold, 2-fold, 3-fold, 4-fold, 5-fold, 10-fold or lower than the expression or activity in the absence of the inhibitor. In embodiments, the inhibitor is a BRAF inhibitor, a MEK inhibitor, or an ERK inhibitor. In embodiments, the inhibitor is an EGFR inhibitor. In embodiments, the inhibitor is a COX-2 inhibitor.

In embodiments, the inhibitor is a MEK inhibitor. In embodiments, the MEK inhibitor is trametinib, binimetinib, cobimetinib, selumetinib, or a combination thereof. In embodiments, the MEK inhibitor is trametinib. In embodiments, the MEK inhibitor is binimetinib. In embodiments, the MEK inhibitor is cobimetinib. In embodiments, the MEK inhibitor is selumetinib. In embodiments, the inhibitor is a BRAF inhibitor. In embodiments, the BRAF inhibitor is encorafenib, vemurafenib, or dabrafenib, or a combination thereof. In embodiments, the BRAF inhibitor is encorafenib. In embodiments, the BRAF inhibitor is vemurafenib. In embodiments, the BRAF inhibitor is dabrafenib. In embodiments, the inhibitor is an ERK inhibitor. In embodiments, the ERK inhibitor is ulixertinib (BVD-523), ravoxertinib (GDC-0994), LY3214996, LTT462, or a combination thereof. In embodiments, the ERK inhibitor is ulixertinib. In embodiments, the ERK inhibitor is ravoxertinib. In embodiments, the ERK inhibitor is LY3214996. In embodiments, the ERK inhibitor is LTT462. In embodiments, the inhibitor is a COX-2 inhibitor. In embodiments, the COX-2 inhibitor is celecoxib, valdecoxib, ketorolac, rofecoxib, or a combination thereof. In embodiments, the COX-2 inhibitor is celecoxib. In embodiments, the COX-2 inhibitor is valdecoxib. In embodiments, the COX-2 inhibitor is ketorolac. In embodiments, the COX-2 inhibitor is rofecoxib. In embodiments, the inhibitor is an EGFR inhibitor. In embodiments, the EGFR inhibitor is panitumumab, cetuximab, gefitinib, erlotinib, neratinib, lapatinib, necitumumab, or a combination thereof. In embodiments, the EGFR inhibitor is panitumumab. In embodiments, the EGFR inhibitor is cetuximab. In embodiments, the EGFR inhibitor is gefitinib. In embodiments, the EGFR inhibitor is erlotinib. In embodiments, the EGFR inhibitor is neratinib. In embodiments, the EGFR inhibitor is lapatinib. In embodiments, the EGFR inhibitor is necitumumab.

The term “trametinib,” also known as MEKINIST®, GSK1120212 or the like, refers in the usual and customary sense, to N-[3-[3-cyclopropyl-5-(2-fluoro-4-iodoanilino)-6,8-dimethyl-2,4,7-trioxopyrido[4,3-d]pyrimidin-1-yl]phenyl]acetamide (CAS Registry number 871700-17-3).

The term “binimetinib,” also known as MEKTOVI®, ARRY-162 or the like, refers in the usual and customary sense, to 5-[(4-bromo-2-fluorophenyl)amino]-4-fluoro-N-(2-hydroxyethoxy)-1-methyl-iH-benzimidazole-6-carboxamide (CAS Registry number 606143-89-9).

The term “cobimetinib,” also known as COTELLIC®, GDC-0973 or the like, refers in the usual and customary sense, to [3,4-difluoro-2-(2-fluoro-4-iodoanilino)phenyl]-[3-hydroxy-3-[(2S)-piperidin-2-yl]azetidin-1-yl]methanone (CAS Registry number 934660-93-2).

The term “selumetinib,” also known as KOSELUGO®, AZD6244 or the like, refers in the usual and customary sense, to 6-(4-bromo-2-chloroanilino)-7-fluoro-N-(2-hydroxyethoxy)-3-methylbenzimidazole-5-carboxamide (CAS Registry number 606143-52-6).

The term “ulixertinib,” also known as BVD-523 or the like, refers in the usual and customary sense, to N-[(1S)-1-(3-chlorophenyl)-2-hydroxyethyl]-4-[5-chloro-2-(propan-2-ylamino)pyridin-4-yl]-1H-pyrrole-2-carboxamide (CAS Registry number 869886-67-9).

The term “ravoxertinib,” also known as GDC-0994 or the like, refers in the usual and customary sense, to 1-[(1S)-1-(4-chloro-3-fluorophenyl)-2-hydroxyethyl]-4-[2-[(2-methylpyrazol-3-yl)amino]pyrimidin-4-yl]pyridin-2-one (CAS Registry number 1453848-26-4).

The term “temuterkib,” also known as LY3214996 or the like, refers in the usual and customary sense, to 6,6-dimnethyl-2-[2-[(2-methylpyrazol-3-yl)amino]pyrimidin-4-yl]-5-(2-morpholin-4-ylethyl)thieno[2,3-c]pyrrol-4-one (CAS Registry number 1951483-29-6).

The term “rineterkib,” also known as LTT462, ERK-IN-1 or the like, refers in the usual and customary sense, to 4-[3-amino-6-[(1 S,3S,4S)-3-fluoro-4-hydroxycyclohexyl]pyrazin-2-yl]-N-[(1 S)-1-(3-bromo-5-fluorophenyl)-2-(nethylamino)ethyl]-2-fluorobenzamide (CAS Registry number 1715025-32-3).

The term “encorafenib,” also known as BRAFTOVI®, GSK1120212 or the like, refers in the usual and customary sense, to methyl N-[(2S)-1-[[4-[3-[5-chloro-2-fluoro-3-(methanesulfonamido)phenyl]-1-propan-2-ylpyrazol-4-yl]pyrimidin-2-yl]amino]propan-2-yl]carbamate (CAS Registry number 1269440-17-6).

The term “vemurafenib,” also known as ZELBORAF® or the like, refers in the usual and customary sense, to N-[3-[5-(4-chlorophenyl)-1H-pyrrolo[2,3-b]pyridine-3-carbonyl]-2,4-difluorophenyl]propane-1-sulfonamide (CAS Registry number 918504-65-1).

The term “dabrafenib,” also known as TAFINLAR®, GSK 436, or the like, refers in the usual and customary sense, to N-[3-[5-(2-Amino-4-pyrimidinyl)-2-(2-methyli-2-propanyl)-1,3-tliazol-4-yl]-2-fluorophenyl]-2,6-difluorobenzenesulfonarmide (CAS Registry number 1195765-45-7).

The term “celecoxib,” also known as CELEBREX®, SC 58635, or the like, refers in the usual and customary sense, to 2,3,5,6-tetradeuterio-4-[5-(4-rnethylphenyl)-3-(trifluoronethyl)pyrazol-1-yl]benzenesulfonanide (CAS Registry number 169590-42-5).

The term “valdecoxib,” also known as BEXTRA®, or the like, refers in the usual and customary sense, to 4-(5-methyl-3-phenyl-1,2-oxazol-4-yl)benzenesulfonamide (CAS Registry number 181695-72-7).

The term “ketorolac,” also known as RS 37619, or the like, refers in the usual and customary sense, to 5-benzoyl-2,3-dihydro-1H-pyrrolizine-1-carboxylic acid (CAS Registry number 74103-06-3).

The term “rofecoxib,” also known as RS 37619, or the like, refers in the usual and customary sense, to 3-(4-methylsulfonylphenyl)-4-phenyl-2H-furan-5-one (CAS Registry number 162011-90-7).

The term “gefitinib,” also known as IRESSA®, ZD1839, or the like, refers in the usual and customary sense, to N-(3-chloro-4-fluorophenyl)-7-methoxy-6-(3-morpholin-4-ylpropoxy)quinazolin-4-amine (CAS Registry number 184475-35-2).

The term “erlotinib,” also known as TARCEVA®, or the like, refers in the usual and customary sense, to N-(3-ethynylphenyl)-6,7-bis(2-mnethoxyethoxy)quinazoiin-4-amine (CAS Registry number 183321-74-6).

The term “neratinib,” also known as HNK-272, or the like, refers in the usual and customary sense, to (E)-N-[4-[3-chloro-4-(pyridin-2-ylmethoxy)anilino]-3-cyano-7-ethoxyquinolin-6-yl]-4-(dimethylamnino)but-2-enamide (CAS Registry number 698387-09-6).

The term “lapatinib,” also known as TYKERB®, or the like, refers in the usual and customary sense, to N-[3-chloro-4-[(3-fluorophenyl)methoxy]phenyl]-6-[5-[(2-methylsulfonylethylamino))methyl]furan-2-yl]quinazolin-4-amine (CAS Registry number 231277-92-2).

The term “dasatinib,” also known as BMS 354825, or the like, refers in the usual and customary sense, to N-(2-chloro-6-mnethylphenyl)-2-[[6-[4-(2-hydroxyethyl)piperazin-1-yl]-2-methylpyrimidin-4-yl]amino]-1,3-thiazole-5-carboxamide (CAS Registry number 302962-49-8).

The term “saracatinib,” also known as AZD0530, or the like, refers in the usual and customary sense, to N-(5-chioro-1,3-benzodioxol-4-yl)-7-[2-(4-methylpiperazin-1-yl)ethoxy]-5-(oxan-4-yloxy)quinazolin-4-amine (CAS Registry number 379231-04-6).

The term “bosutinib,” also known as BOSULIF®, AZD0530, or the like, refers in the usual and customary sense, to 4-(2,4-dichloro-5-methoxyanilino)-6-methoxy-7-[3-(4-methylpiperazin-1-yl)propoxy]quinoline-3-carbonitrile (CAS Registry number 380843-75-4).

The term “ponatinib,” also known as AP24534, ICLUSIG®, or the like, refers in the usual and customary sense, to 3-(2-imidazo[1,2-b]pyridazin-3-ylethynvl)-4-netil-N-[4-[(4-methylpiperazin-1-yl)methyl]-3-(trifluoromethyl)phenyl]benzamide (CAS Registry number 943319-70-8).

The term “imatinib,” also known as GLEE.VEC®, or the like, refers in the usual and customary sense, to 4-[(4-methylpiperazin-1-yl)methyl]-N-[4-methyl-3-[(4-pyridin-3-ylpyrimidin-2-yl)amino]phenyl]benzamide (CAS Registry number 152459-95-5).

In embodiments, the EGFR inhibitor is an anti-EGFR antibody or fragment thereof that specifically binds to EGFR. In embodiments, the anti-EGFR antibody is cetuximab or a fragment thereof. In embodiments, the anti-EGFR antibody includes the CDR sequences of cetuximab (i.e. the sequences of CDR1, CDR2, and CDR3). In embodiments, cetuximab includes a heavy chain variable domain including the sequence of SEQ ID NO:24, and a light chain variable domain including the sequence of SEQ ID NO:25. In embodiments, the anti-EGFR antibody is panitumumab or a fragment thereof. In embodiments, the anti-EGFR antibody includes the CDR sequences of panitumumab (i.e. the sequences of CDR1, CDR2, and CDR3). In embodiments, panitumumab includes a heavy chain variable domain including the sequence of SEQ ID NO:26, and a light chain variable domain including the sequence of SEQ ID NO:27. In embodiments, the anti-EGFR antibody is necitumumab or a fragment thereof. In embodiments, the anti-EGFR antibody includes the CDR sequences of necitumumab (i.e. the sequences of CDR1, CDR2, and CDR3). In embodiments, necitumumab includes a heavy chain variable domain including the sequence of SEQ ID NO:28, and a light chain variable domain including the sequence of SEQ ID NO:29.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.). Expression can be detected by, for example, measuring the enzymatic activity of the polypeptide. For example, expression can be detected by measuring the phospho-catalytic of activity of a kinase (e.g. BRAF).

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

A “control” or “standard control” refers to a sample, measurement, or value that serves as a reference, usually a known reference, for comparison to a test sample, measurement, or value. For example, a test sample can be taken from a patient suspected of having a given disease (e.g. cancer) and compared to a known normal (non-diseased) individual (e.g. a standard control subject). A standard control can also represent an average measurement or value gathered from a population of similar individuals (e.g. standard control subjects) that do not have a given disease (i.e. standard control population), e.g., healthy individuals with a similar medical background, same age, weight, etc. A standard control value can also be obtained from the same individual, e.g. from an earlier-obtained sample from the patient prior to disease onset. For example, a control can be devised to compare therapeutic benefit based on pharmacological data (e.g., half-life) or therapeutic measures (e.g., comparison of side effects). Controls are also valuable for determining the significance of data. For example, if values for a given parameter are widely variant in controls, variation in test samples will not be considered as significant. One of skill will recognize that standard controls can be designed for assessment of any number of parameters (e.g. RNA levels, protein levels, specific cell types, specific bodily fluids, specific tissues, etc). One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

“Patient”, “subject” or “subject in need thereof” refers to a living organism suffering from or prone to a disease or condition (e.g. a BRAF intrinsically resistant cancer (e.g. colorectal cancer, etc.)) that can be treated by administration of a combination therapy as provided herein. Non-limiting examples include humans, other mammals, bovines, rats, mice, dogs, monkeys, goat, sheep, cows, deer, and other non-mammalian animals. In some embodiments, a patient is human.

The terms “disease” or “condition” refer to a state of being or health status of a patient or subject capable of being treated with the compounds or methods provided herein. The disease may be a cancer. The disease may be an autoimmune disease. The disease may be an inflammatory disease. The disease may be an infectious disease. In some further instances, “cancer” refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), or multiple myeloma.

As used herein, the term “cancer” refers to all types of cancer, neoplasm or malignant tumors found in mammals (e.g., humans), including leukemia, lymphoma, carcinomas and sarcomas. Cancers that may be treated with a compound or method provided herein include cancer of the thyroid, endocrine system, brain, breast, cervix, colon, head and neck, liver, kidney, lung, non-small cell lung, melanoma, mesothelioma, ovary, sarcoma, stomach, uterus medulloblastoma, colorectal cancer, or pancreatic cancer. Additional examples include Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, lymphomas, thyroid cancer, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer. In embodiments, the cancer expresses BRAF^{V600E}. In embodiments, the cancer is a BRAF intrinsically resistant cancer as provided herein.

Exemplary cancers that may be treated with a compound or method provided herein include colorectal cancer, thyroid cancer, glioblastoma, sarcoma, and hepto-sarcoma. In embodiments, the cancer is colorectal cancer. In embodiments, the cancer is thyroid cancer. In embodiments, the cancer is glioblastoma. In embodiments, the cancer is sarcoma. In embodiments, the cancer is hepto-sarcoma. Additional examples include cholangiocarcinoma, chronic lymphocytic leukemia, Erdheim-Chester disease, ganglioglioma, gastrointestinal stromal tumor (GIST), hairy cell leukemia, kidney cancer, lung cancer adenocarcinoma, Langerhans cell histiocytosis, melanoma, multiple myeloma, ovarian cancer, pancreatic cancer, pilocystic astrocytoma, pleomorphic xanthoastrocytoma, prostate cancer, and papillary thyroid cancer.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., colon or rectum, which site is referred to as a primary tumor, e.g., primary colon cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if colorectal cancer metastasizes to the liver, the secondary tumor at the site of the liver consists of abnormal colorectal cells and not abnormal liver cells. The secondary tumor in the liver is referred to a colorectal lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic colorectal cancer refers to a disease in a subject with or with a history of a primary colorectal tumor and with one or more secondary tumors at a second location or multiple locations, e.g., in the liver.

Cancer model organism, as used herein, is an organism exhibiting a phenotype indicative of cancer, or the activity of cancer causing elements, within the organism. The term cancer is defined above. A wide variety of organisms may serve as cancer model organisms, and include for example, cancer cells and mammalian organisms such as rodents (e.g. mouse or rat) and primates (such as humans). Cancer cell lines are widely understood by those skilled in the art as cells exhibiting phenotypes or genotypes similar to in vivo cancers. Cancer cell lines as used herein includes cell lines from animals (e.g. mice) and from humans.

The term “associated” or “associated with” in the context of a substance or substance activity or function associated with a disease means that the disease (e.g. BRAF intrinsically resistant cancer) is caused by (in whole or in part), or a symptom of the disease is caused by (in whole or in part) the substance or substance activity or function. As used herein, what is described as being associated with a disease, if a causative agent, could be a target for treatment of the disease.

The term “aberrant” as used herein refers to different from normal. When used to describe enzymatic activity or protein function, aberrant refers to activity or function that is greater or less than a normal control or the average of normal non-diseased control samples. Aberrant activity may refer to an amount of activity that results in a disease, wherein returning the aberrant activity to a normal or non-disease-associated amount (e.g. by administering a compound or using a method as described herein), results in reduction of the disease or one or more disease symptoms.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is no prophylactic treatment.

The term “prevent” refers to a decrease in the occurrence of disease symptoms in a patient. As indicated above, the prevention may be complete (no detectable symptoms) or partial, such that fewer symptoms are observed than would likely occur absent treatment.

As used herein, the term “administering” is used in accordance with its plain and ordinary meaning and includes oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

“Co-administer” it is meant that a composition described herein is administered at the same time, just prior to, or just after the administration of one or more additional therapies. The compounds provided herein can be administered alone or can be coadministered to the patient. Co-administration is meant to include simultaneous or sequential administration of the compounds individually or in combination (more than one compound). Thus, the preparations can also be combined, when desired, with other active substances (e.g., to reduce metabolic degradation). The compositions of the present disclosure can be delivered transdermally, by a topical route, or formulated as applicator sticks, solutions, suspensions, emulsions, gels, creams, ointments, pastes, jellies, paints, powders, and aerosols.

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

A “therapeutic agent”, “therapeutic” or “therapy” as used herein refers to an agent (e.g., compound or composition described herein) that when administered to a subject will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms or the intended therapeutic effect, e.g., treatment or amelioration of an injury, disease, pathology or condition, or their symptoms including any objective or subjective parameter of treatment such as abatement; remission; diminishing of symptoms or making the injury, pathology or condition more tolerable to the patient; slowing in the rate of degeneration or decline; making the final point of degeneration less debilitating; or improving a patient's physical or mental well-being. In embodiments, the therapeutic is a combination therapeutic/combination therapy including a plurality of agents (e.g. a BRAF axis inhibitor and a COX-inhibitor; a BRAF axis inhibitor, EGFR inhibitor and a COX-inhibitor etc.). In embodiments, the agents of the combination therapy may be administered sequentially or simultaneously. In embodiments, the agents of the combination therapy are anti-cancer agents.

“Anti-cancer agent” and “anticancer agent” are used in accordance with their plain ordinary meaning and refers to a composition (e.g. compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer. In some embodiments, the anti-cancer agent is a BRAF inhibitor, a MEK inhibitor, an ERK inhibitor, or a COX-2 inhibitor. In embodiments, the anti-cancer agent is an EGFR inhibitor. In embodiments, the anti-cancer agent is a targeted anti-cancer therapeutic (e.g. targeted therapy). As used herein, “targeted anti-cancer therapeutic” or “targeted therapy” refers to a compound that targets a specific protein or gene associated with the cancer. For example, the targeted anti-cancer therapeutic may target BRAF protein. For example, the targeted anti-cancer therapeutic may target a nucleic acid encoding BRAF protein. In embodiments, the targeted anti-cancer therapeutic targets a mutated BRAF protein. In embodiments, the targeted anti-cancer therapeutic targets a nucleic acid encoding a mutated BRAF protein. In embodiments, the targeted anti-cancer therapeutic targets BRAF^{V600E}. In embodiments, the targeted anti-cancer therapeutic targets BRAF^{V600K}. In embodiments, the targeted anti-cancer therapeutic targets BRAF^{V600D}. In embodiments, the targeted anti-cancer therapeutic targets BRAF^{V600R}. In embodiments, the targeted anti-cancer therapeutic targets BRAF^{K6001E}. In embodiments, the targeted anti-cancer therapeutic targets BRAF^{K6001N}. I In embodiments, the targeted anti-cancer therapeutic targets BRAF^{L597S}. In embodiments, the targeted anti-cancer therapeutic targets BRAF^{L597R}. In embodiments, the targeted anti-cancer therapeutic targets BRAF^{L597Q}.

A “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” In the methods described herein, an effective amount of a BRAF axis inhibitor is combined with an effective amount of a COX-2 inhibitor. In these methods, the effective amount of the BRAF axis inhibitor is an amount effective to accomplish the stated purpose of the method in combination with an effective amount of a COX-2 inhibitor. Likewise, an effective amount of a COX-2 inhibitor is an amount effective to accomplish the stated purpose of the method in combination with an effective amount of BRAF inhibitor. In this context, the effective amount of BRAF axis inhibitor and the effective amount of COX-2 inhibitor may be referred to as a “combined effective amount” of BRAF axis inhibitor and COX-2 inhibitor. For the methods described herein, in embodiments, an effective amount of a BRAF axis inhibitor is combined with an effective amount of an EGFR inhibitor and an effective amount of a COX-2 inhibitor. In these methods, the effective amount of the BRAF axis inhibitor is an amount effective to accomplish the stated purpose of the method in combination with an effective amount of an EGFR inhibitor and an effective amount of a COX-2 inhibitor. Likewise, an effective amount of an EGFR inhibitor is an amount effective to accomplish the stated purpose of the method in combination with an effective amount of BRAF inhibitor and effective amount of COX-2 inhibitor. Likewise, an effective amount of a COX-2 inhibitor is an amount effective to accomplish the stated purpose of the method in combination with an effective amount of BRAF inhibitor and an effective amount of EGFR inhibitor. In this context, the effective amount of BRAF axis inhibitor, the effective amount of EGFR inhibitor, and the effective amount of COX-2 inhibitor may be referred to as a “combined effective amount” of BRAF axis inhibitor, EGFR inhibitor, and COX-2 inhibitor. An example of an “effective amount” or “combined effective amount” is an amount sufficient to contribute to the treatment, prevention, or reduction of a symptom or symptoms of a disease, which could also be referred to as a “therapeutically effective amount” or “therapeutically combined effective amount.” A “reduction” of a symptom or symptoms (and grammatical equivalents of this phrase) means decreasing of the severity or frequency of the symptom(s), or elimination of the symptom(s). The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist.

For any compound described herein, the therapeutically effective amount can be initially determined from cell culture assays. Target concentrations will be those concentrations of active compound(s) that are capable of achieving the methods described herein, as measured using the methods described herein or known in the art.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control. As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Methods of Treatment

Provided herein, inter alia, are methods of treating cancer in a subject, including administering a combination treatment including a BRAF axis inhibitor and a COX-2 inhibitor. In embodiments, the combination treatment includes a BRAF axis inhibitor, a COX-2 inhibitor, and an EGFR inhibitor. Applicant has demonstrated that the methods provided herein including embodiments thereof are effective for treating cancers that express a mutated BRAF protein, for example, BRAF^{V600E}. In embodiments, the cancer is a BRAF resistant cancer. A “BRAF resistant cancer” as used herein refers to a cancer that is nonresponsive or poorly responsive to a BRAF axis inhibitor. Anti-cancer response to the BRAF axis inhibitor can be measured by, for example, cancer cell death, inhibition or slowing of tumor growth, lack of progression of the cancer, prolonged survival, or desired or beneficial clinical results. An anti-cancer response may be measured in vitro (e.g. in a cancer cell culture), for example, to measure cancer cell viability/proliferation, apoptosis (cancer cell death), cellular senescence, invasion and migration, etc. An anti-cancer response can further be measured in vivo, for example in a cancer model organism, a patient derived xenograph mouse model, etc. In embodiments, a BRAF resistant cancer has less than about 40%, 30%, 20%, 18%, 16%, 14%, 12%, 10%, 8%, 6%, 4%, 2% or 1% anti-cancer response to the BRAF axis inhibitor. In embodiments, a BRAF resistant cancer has less than about 40% anti-cancer response to the BRAF axis inhibitor. In embodiments, a BRAF resistant cancer has less than about 30% anti-cancer response to the BRAF axis inhibitor. In embodiments, a BRAF resistant cancer has less than about 20% anti-cancer response to the BRAF axis inhibitor. In embodiments, a BRAF resistant cancer has less than about 18% anti-cancer response to the BRAF axis inhibitor. In embodiments, a BRAF resistant cancer has less than about 16% anti-cancer response to the BRAF axis inhibitor. In embodiments, a BRAF resistant cancer has less than about 14% anti-cancer response to the BRAF axis inhibitor. In embodiments, a BRAF resistant cancer has less than about 12% anti-cancer response to the BRAF axis inhibitor. In embodiments, a BRAF resistant cancer has less than about 10% anti-cancer response to the BRAF axis inhibitor. In embodiments, a BRAF resistant cancer has less than about 8% anti-cancer response to the BRAF axis inhibitor. In embodiments, a BRAF resistant cancer has less than about 6% anti-cancer response to the BRAF axis inhibitor. In embodiments, a BRAF resistant cancer has less than about 4% anti-cancer response to the BRAF axis inhibitor. In embodiments, a BRAF resistant cancer has less than about 3% anti-cancer response to the BRAF axis inhibitor. In embodiments, a BRAF resistant cancer has less than about 2% anti-cancer response to the BRAF axis inhibitor. In embodiments, a BRAF resistant cancer has less than about 1% anti-cancer response to the BRAF axis inhibitor. In embodiments, a BRAF resistant cancer is nonresponsive to the BRAF axis inhibitor.

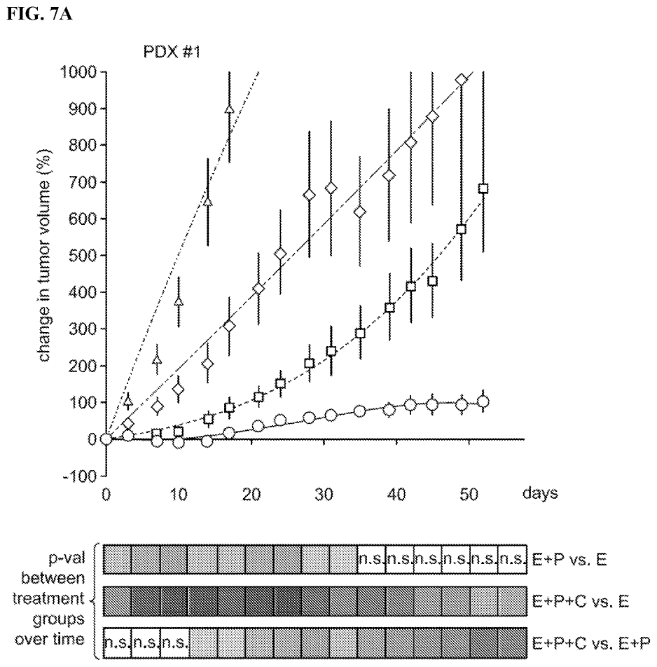

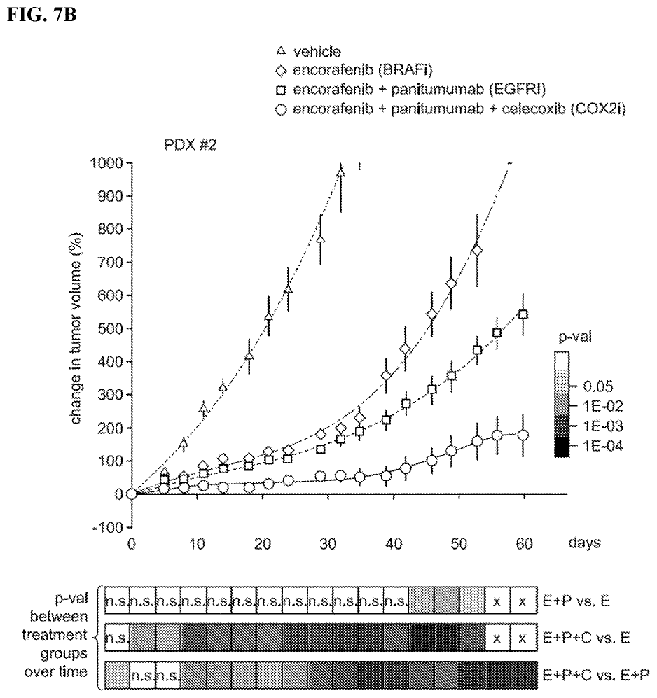

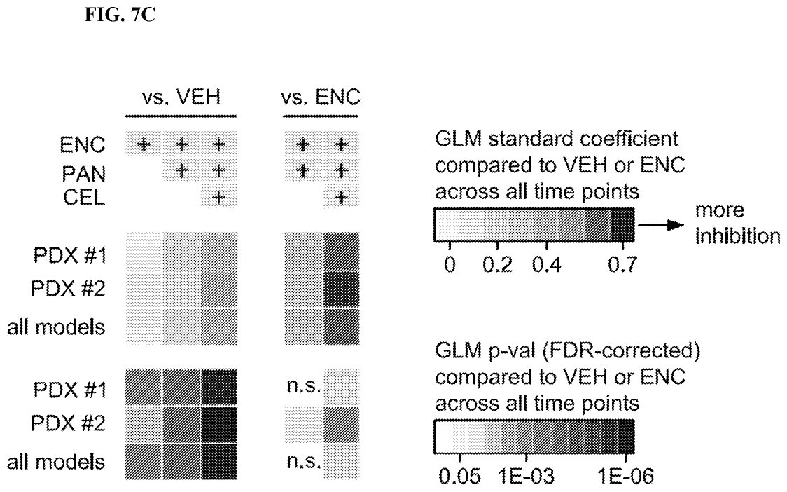

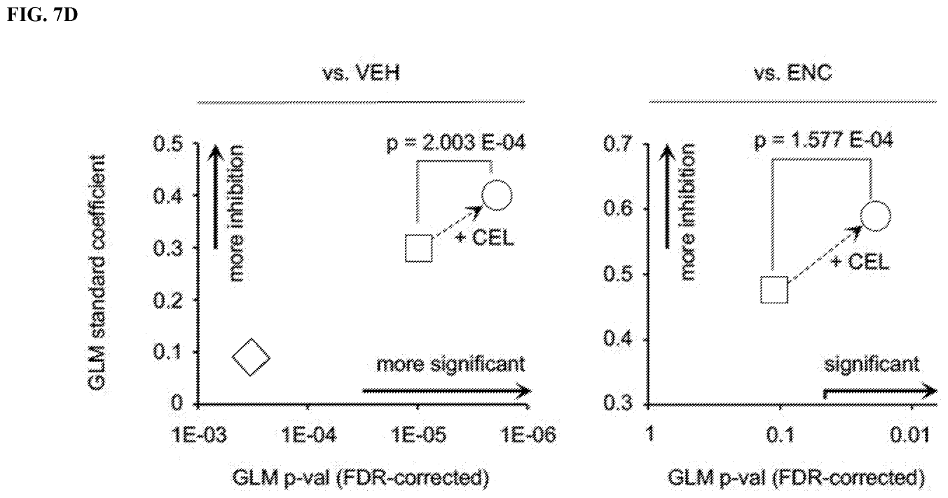

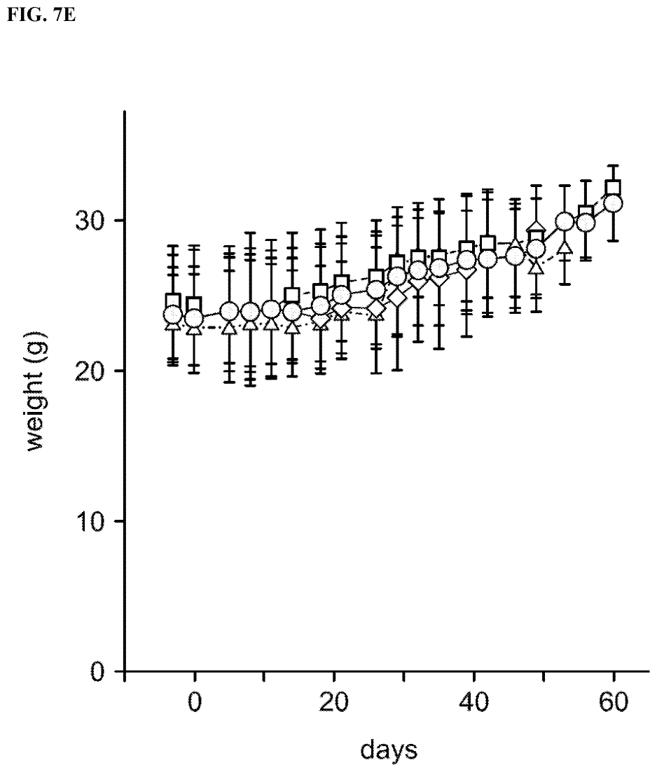

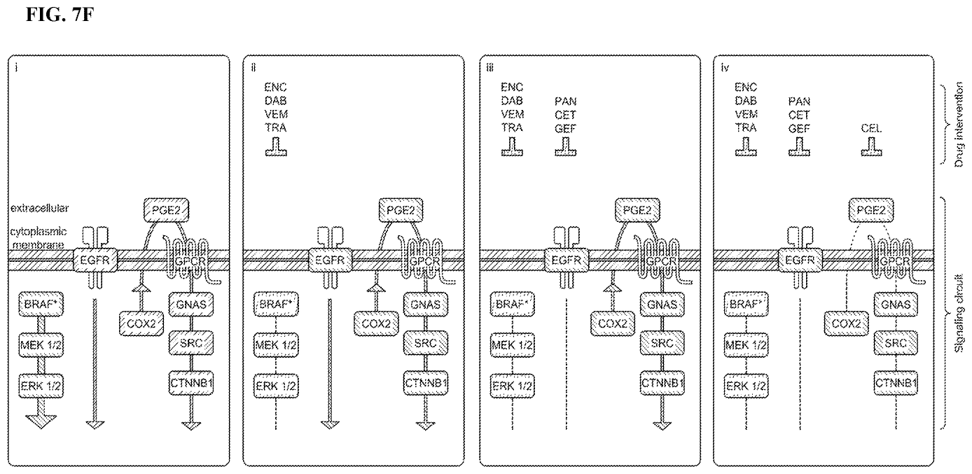

The methods provided herein are effective for treating BRAF axis intrinsically resistant cancers, which have previously been be shown to be nonresponsive or poorly responsive to combination treatments known in the art, for example, a BRAF inhibitor and MEK inhibitor, or a BRAF inhibitor and EGFR inhibitor. Without wishing to be bound by scientific theory, Applicant has discovered that BRAF axis inhibitors activate an adaptive response in BRAF intrinsically resistant cancer cells, thereby resulting in nonresponse, low response, or resistance to the therapeutic (e.g. BRAF inhibitor, BRAF inhibitor and MEK inhibitor, BRAF inhibitor and EGFR inhibitor, etc.). Without wishing to be bound by scientific theory, Applicant discovered that, surprisingly, the adaptive response to the BRAF axis inhibitor is independent of the BRAF and EGFR signaling pathways. See, for example, FIG. 7F. Applicant demonstrates herein that administration of a COX-2 inhibitor in combination with a BRAF axis inhibitor decreases or inhibits the adaptive response to the BRAF axis inhibitor.

As used herein, “B-Raf intrinsically resistant cancer” or “BRAF intrinsically resistant cancer” refers to a cancer that is resistant to a BRAF axis inhibitor prior to the initial treatment with the BRAF axis inhibitor (e.g. the cancer has pre-existing resistance to the treatment). For example, a BRAF intrinsically resistant cancer (e.g. colorectal cancer) may not previously be sensitive to the BRAF axis inhibitor prior to starting treatment with the BRAF axis inhibitor. In embodiments, a BRAF intrinsically resistant cancer may have a lack of anti-cancer response to an initial treatment with the BRAF axis inhibitor. Anti-cancer response to the BRAF axis inhibitor can be measured by, for example, cancer cell death, inhibition or slowing of tumor growth, lack of progression of the cancer, prolonged survival, or desired or beneficial clinical results. In embodiments, a BRAF intrinsically resistant cancer has less than about 40% anti-cancer response to the BRAF axis inhibitor. In embodiments, a BRAF intrinsically resistant cancer has less than about 40%, 30%, 20%, 18%, 16%, 14%, 12%, 10%, 8%, 6%, 4%, 2% or 1% anti-cancer response to the BRAF axis inhibitor. In embodiments, a BRAF intrinsically resistant cancer is nonresponsive to the BRAF axis inhibitor.

In embodiments, resistance to a BRAF axis inhibitor is resistance to a BRAF inhibitor monotherapy (e.g. one BRAF inhibitor with no other BRAF inhibitors). In embodiments, a BRAF intrinsically resistant cancer has less than about 40%, 30%, 20%, 18%, 16%, 14%, 12%, 10%, 8%, 6%, 4%, 2% or 1% anti-cancer response to the BRAF inhibitor monotherapy. In embodiments, a BRAF intrinsically resistant cancer has less than about 40% anti-cancer response to the BRAF inhibitor monotherapy. In embodiments, a BRAF intrinsically resistant cancer has less than about 30% anti-cancer response to the BRAF inhibitor monotherapy. In embodiments, a BRAF intrinsically resistant cancer has less than about 20% anti-cancer response to the BRAF inhibitor monotherapy. In embodiments, a BRAF intrinsically resistant cancer has less than about 20% anti-cancer response to the BRAF inhibitor monotherapy. In embodiments, a BRAF intrinsically resistant cancer has less than about 18% anti-cancer response to the BRAF inhibitor monotherapy. In embodiments, a BRAF intrinsically resistant cancer has less than about 16% anti-cancer response to the BRAF inhibitor monotherapy. In embodiments, a BRAF intrinsically resistant cancer has less than about 14% anti-cancer response to the BRAF inhibitor monotherapy. In embodiments, a BRAF intrinsically resistant cancer has less than about 12% anti-cancer response to the BRAF inhibitor monotherapy. In embodiments, a BRAF intrinsically resistant cancer has less than about 10% anti-cancer response to the BRAF inhibitor monotherapy. In embodiments, a BRAF intrinsically resistant cancer has less than about 8% anti-cancer response to the BRAF inhibitor monotherapy. In embodiments, a BRAF intrinsically resistant cancer has less than about 6% anti-cancer response to the BRAF inhibitor monotherapy. In embodiments, a BRAF intrinsically resistant cancer has less than about 4% anti-cancer response to the BRAF inhibitor monotherapy. In embodiments, a BRAF intrinsically resistant cancer has less than about 2% anti-cancer response to the BRAF inhibitor monotherapy. In embodiments, a BRAF intrinsically resistant cancer has less than about 1% anti-cancer response to the BRAF inhibitor monotherapy. In embodiments, a BRAF intrinsically resistant cancer is nonresponsive to the BRAF inhibitor monotherapy.

In embodiments, resistance to a BRAF axis inhibitor is resistance to a BRAF inhibitor combined with a MEK inhibitor or ERK inhibitor. In embodiments, a BRAF intrinsically resistant cancer has less than about 40%, 30%, 20%, 18%, 16%, 14%, 12%, 10%, 8%, 6%, 4%, 2% or 1% anti-cancer response to the BRAF inhibitor combined with a MEK inhibitor or ERK inhibitor. In embodiments, a BRAF intrinsically resistant cancer has less than about 40% anti-cancer response to the BRAF inhibitor combined with a MEK inhibitor or ERK inhibitor. In embodiments, a BRAF intrinsically resistant cancer has less than about 30% anti-cancer response to the BRAF inhibitor combined with a MEK inhibitor or ERK inhibitor. In embodiments, a BRAF intrinsically resistant cancer has less than about 20% anti-cancer response to the BRAF inhibitor combined with a MEK inhibitor or ERK inhibitor. In embodiments, a BRAF intrinsically resistant cancer has less than about 18% anti-cancer response to the BRAF inhibitor combined with a MEK inhibitor or ERK inhibitor. In embodiments, a BRAF intrinsically resistant cancer has less than about 16% anti-cancer response to the BRAF inhibitor combined with a MEK inhibitor or ERK inhibitor. In embodiments, a BRAF intrinsically resistant cancer has less than about 14% anti-cancer response to the BRAF inhibitor combined with a MEK inhibitor or ERK inhibitor. In embodiments, a BRAF intrinsically resistant cancer has less than about 12% anti-cancer response to the BRAF inhibitor combined with a MEK inhibitor or ERK inhibitor. In embodiments, a BRAF intrinsically resistant cancer has less than about 10% anti-cancer response to the BRAF inhibitor combined with a MEK inhibitor or ERK inhibitor. In embodiments, a BRAF intrinsically resistant cancer has less than about 8% anti-cancer response to the BRAF inhibitor combined with a MEK inhibitor or ERK inhibitor. In embodiments, a BRAF intrinsically resistant cancer has less than about 6% anti-cancer response to the BRAF inhibitor combined with a MEK inhibitor or ERK inhibitor. In embodiments, a BRAF intrinsically resistant cancer has less than about 4% anti-cancer response to the BRAF inhibitor combined with a MEK inhibitor or ERK inhibitor. In embodiments, a BRAF intrinsically resistant cancer has less than about 2% anti-cancer response to the BRAF inhibitor combined with a MEK inhibitor or ERK inhibitor. In embodiments, a BRAF intrinsically resistant cancer has less than about 1% anti-cancer response to the BRAF inhibitor combined with a MEK inhibitor or ERK inhibitor. In embodiments, a BRAF intrinsically resistant cancer is nonresponsive to the BRAF inhibitor combined with a MEK inhibitor or ERK inhibitor.

In embodiments, resistance to a BRAF axis inhibitor is resistance to a BRAF axis inhibitor and an EGFR inhibitor (e.g. encorafenib and cetuximab, etc). In embodiments, a BRAF intrinsically resistant cancer has less than about 40%, 30%, 20%, 18%, 16%, 14%, 12%, 10%, 8%, 6%, 4%, 2% or 1% anti-cancer response to the BRAF axis inhibitor and EGFR inhibitor. In embodiments, a BRAF intrinsically resistant cancer has less than about 40% anti-cancer response to the BRAF axis inhibitor and EGFR inhibitor. In embodiments, a BRAF intrinsically resistant cancer has less than about 30% anti-cancer response to the BRAF axis inhibitor and EGFR inhibitor. In embodiments, a BRAF intrinsically resistant cancer has less than about 20% anti-cancer response to the BRAF axis inhibitor and EGFR inhibitor. In embodiments, a BRAF intrinsically resistant cancer has less than about 20% anti-cancer response to the BRAF axis inhibitor and EGFR inhibitor. In embodiments, a BRAF intrinsically resistant cancer has less than about 18% anti-cancer response to the BRAF axis inhibitor and EGFR inhibitor. In embodiments, a BRAF intrinsically resistant cancer has less than about 16% anti-cancer response to the BRAF axis inhibitor and EGFR inhibitor. In embodiments, a BRAF intrinsically resistant cancer has less than about 14% anti-cancer response to the BRAF axis inhibitor and EGFR inhibitor. In embodiments, a BRAF intrinsically resistant cancer has less than about 12% anti-cancer response to the BRAF axis inhibitor and EGFR inhibitor. In embodiments, a BRAF intrinsically resistant cancer has less than about 10% anti-cancer response to the BRAF axis inhibitor and EGFR inhibitor. In embodiments, a BRAF intrinsically resistant cancer has less than about 8% anti-cancer response to the BRAF axis inhibitor and EGFR inhibitor. In embodiments, a BRAF intrinsically resistant cancer has less than about 6% anti-cancer response to the BRAF axis inhibitor and EGFR inhibitor. In embodiments, a BRAF intrinsically resistant cancer has less than about 4% anti-cancer response to the BRAF axis inhibitor and EGFR inhibitor. In embodiments, a BRAF intrinsically resistant cancer has less than about 2% anti-cancer response to the BRAF axis inhibitor and EGFR inhibitor. In embodiments, a BRAF intrinsically resistant cancer has less than about 1% anti-cancer response to the BRAF axis inhibitor and EGFR inhibitor. In embodiments, a BRAF intrinsically resistant cancer is nonresponsive to the BRAF axis inhibitor and EGFR inhibitor.

In contrast, a BRAF acquired resistant cancer is initially sensitive to a BRAF axis inhibitor before developing resistance to the BRAF axis inhibitor. For example, one or more acquired mutations resulting in BRAF axis inhibitor resistant phenotype may cause a cancer that is initially sensitive to a BRAF axis inhibitor to become a BRAF acquired resistant cancer. A BRAF acquired resistant cancer therefore may develop resistance to the BRAF axis inhibitor due to a mutation that occurs after initial treatment with the BRAF axis inhibitor. In embodiments, the mutation is BRAF^{V600E}.

As used herein, “B-Raf axis” or “BRAF axis” refers to components of the BRAF signaling pathway, including BRAF, MEK, and ERK. Thus, “B-Raf axis inhibitor” or “BRAF axis inhibitor” refers to a compound that decreases or inhibits the expression or activity of one or more components of the BRAF axis (e.g. BRAF, MEK, ERK). In embodiments, the BRAF axis inhibitor is a compound that binds BRAF, MEK, or ERK. In embodiments, the BRAF axis inhibitor includes a plurality of BRAF axis inhibitors, wherein each of the plurality BRAF axis inhibitors targets a different component of the BRAF signaling pathway (e.g. BRAF and MEK, BRAF and ERK, etc.) to increase or enhance inhibitory effects on the BRAF signaling pathway. In embodiments, the BRAF axis inhibitor includes a single BRAF axis inhibitor. In embodiments, the BRAF axis inhibitor decreases or inhibits the production or activity of one or more components downstream of the BRAF axis.

As described above, Applicant discovered that administration of a combination therapy including a COX-2 inhibitor and a BRAF axis inhibitor is effective for treating BRAF intrinsically resistant cancers. In embodiments, the combination therapy includes a COX-2 inhibitor, a BRAF axis inhibitor, and an EGFR inhibitor. As described throughout the specification, Applicant discovered that administration of a BRAF axis inhibitor upregulates or activates an adaptive response (e.g. survival mechanism) in BRAF intrinsically resistant cancers. For example, administration of a BRAF axis inhibitor may increase or upregulate SRC activity thereby resulting in increased or upregulated beta-catenin activity, a transcription factor involved in survival mechanisms in BRAF intrinsically resistant cancers. Applicant demonstrated that administering a COX-2 inhibitor, an upstream regulator of SRC, increases the anti-cancer response of cancer cells to BRAF axis inhibitors. “COX-2 inhibitor” is used in accordance to its plain ordinary meaning in the art and refers to a compound that decreases or inhibits the production or activity of COX-2 or a component downstream of COX-2, including PGE2, GNAS, Src, or beta-catenin. Applicant further demonstrated that combination therapies, including triplet and quadruple therapies including a BRAF axis inhibitor and a COX-2 inhibitor demonstrate low toxicity while effectively killing cancer cells.

In embodiments, the anti-cancer response to a therapeutic including a BRAF axis inhibitor and a COX-2 inhibitor is increased by at least about 40%, 50%, 60%, 70%, 80%, 90%, or 100% compared to a therapeutic including a BRAF axis inhibitor without a COX-2 inhibitor. Anti-cancer response can be measured by, for example, cancer cell death, inhibition or slowing of tumor growth, lack of progression of the cancer, prolonged survival, or desired or beneficial clinical results. In embodiments, anti-cancer response to a therapeutic including a BRAF axis inhibitor and a COX-2 inhibitor is increased by at least about 40% compared to a therapeutic including a BRAF axis inhibitor without a COX-2 inhibitor. In embodiments, anti-cancer response to a therapeutic including a BRAF axis inhibitor and a COX-2 inhibitor is increased by at least about 50% compared to a therapeutic including a BRAF axis inhibitor without a COX-2 inhibitor. In embodiments, anti-cancer response to a therapeutic including a BRAF axis inhibitor and a COX-2 inhibitor is increased by at least about 60% compared to a therapeutic including a BRAF axis inhibitor without a COX-2 inhibitor. In embodiments, anti-cancer response to a therapeutic including a BRAF axis inhibitor and a COX-2 inhibitor is increased by at least about 70% compared to a therapeutic including a BRAF axis inhibitor without a COX-2 inhibitor. In embodiments, anti-cancer response to a therapeutic including a BRAF axis inhibitor and a COX-2 inhibitor is increased by at least about 80% compared to a therapeutic including a BRAF axis inhibitor without a COX-2 inhibitor. In embodiments, anti-cancer response to a therapeutic including a BRAF axis inhibitor and a COX-2 inhibitor is increased by at least about 85% compared to a therapeutic including a BRAF axis inhibitor without a COX-2 inhibitor. In embodiments, anti-cancer response to a therapeutic including a BRAF axis inhibitor and a COX-2 inhibitor is increased by at least about 90% compared to a therapeutic including a BRAF axis inhibitor without a COX-2 inhibitor. In embodiments, anti-cancer response to a therapeutic including a BRAF axis inhibitor and a COX-2 inhibitor is increased by at least about 91% compared to a therapeutic including a BRAF axis inhibitor without a COX-2 inhibitor. In embodiments, anti-cancer response to a therapeutic including a BRAF axis inhibitor and a COX-2 inhibitor is increased by at least about 92% compared to a therapeutic including a BRAF axis inhibitor without a COX-2 inhibitor. In embodiments, anti-cancer response to a therapeutic including a BRAF axis inhibitor and a COX-2 inhibitor is increased by at least about 93% compared to a therapeutic including a BRAF axis inhibitor without a COX-2 inhibitor. In embodiments, anti-cancer response to a therapeutic including a BRAF axis inhibitor and a COX-2 inhibitor is increased by at least about 94% compared to a therapeutic including a BRAF axis inhibitor without a COX-2 inhibitor. In embodiments, anti-cancer response to a therapeutic including a BRAF axis inhibitor and a COX-2 inhibitor is increased by at least about 95% compared to a therapeutic including a BRAF axis inhibitor without a COX-2 inhibitor. In embodiments, anti-cancer response to a therapeutic including a BRAF axis inhibitor and a COX-2 inhibitor is increased by at least about 96% compared to a therapeutic including a BRAF axis inhibitor without a COX-2 inhibitor. In embodiments, anti-cancer response to a therapeutic including a BRAF axis inhibitor and a COX-2 inhibitor is increased by at least about 97% compared to a therapeutic including a BRAF axis inhibitor without a COX-2 inhibitor. In embodiments, anti-cancer response to a therapeutic including a BRAF axis inhibitor and a COX-2 inhibitor is increased by at least about 98% compared to a therapeutic including a BRAF axis inhibitor without a COX-2 inhibitor. In embodiments, anti-cancer response to a therapeutic including a BRAF axis inhibitor and a COX-2 inhibitor is increased by at least about 99% compared to a therapeutic including a BRAF axis inhibitor without a COX-2 inhibitor. In embodiments, anti-cancer response to a therapeutic including a BRAF axis inhibitor and a COX-2 inhibitor is increased by at least about 100% compared to a therapeutic including a BRAF axis inhibitor without a COX-2 inhibitor.

In embodiments, anti-cancer response to a therapeutic including a BRAF axis inhibitor and a COX-2 inhibitor is increased by at least about 0.1-fold, 0.2-fold, 0.4-fold, 0.6-fold, 0.8-fold, 1-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 100-fold, 150-fold, or 200-fold compared to a therapeutic including a BRAF axis inhibitor without a COX-2 inhibitor. In embodiments, anti-cancer response to a therapeutic including a BRAF axis inhibitor and a COX-2 inhibitor is increased by at least about 0.1-fold compared to a therapeutic including a BRAF axis inhibitor without a COX-2 inhibitor. In embodiments, anti-cancer response to a therapeutic including a BRAF axis inhibitor and a COX-2 inhibitor is increased by at least about 0.2-fold compared to a therapeutic including a BRAF axis inhibitor without a COX-2 inhibitor. In embodiments, anti-cancer response to a therapeutic including a BRAF axis inhibitor and a COX-2 inhibitor is increased by at least about 0.4-fold compared to a therapeutic including a BRAF axis inhibitor without a COX-2 inhibitor. In embodiments, anti-cancer response to a therapeutic including a BRAF axis inhibitor and a COX-2 inhibitor is increased by at least about 0.6-fold compared to a therapeutic including a BRAF axis inhibitor without a COX-2 inhibitor. In embodiments, anti-cancer response to a therapeutic including a BRAF axis inhibitor and a COX-2 inhibitor is increased by at least about 0.8-fold compared to a therapeutic including a BRAF axis inhibitor without a COX-2 inhibitor. In embodiments, anti-cancer response to a therapeutic including a BRAF axis inhibitor and a COX-2 inhibitor is increased by at least about 1-fold compared to a therapeutic including a BRAF axis inhibitor without a COX-2 inhibitor. In embodiments, anti-cancer response to a therapeutic including a BRAF axis inhibitor and a COX-2 inhibitor is increased by at least about 2-fold compared to a therapeutic including a BRAF axis inhibitor without a COX-2 inhibitor. In embodiments, anti-cancer response to a therapeutic including a BRAF axis inhibitor and a COX-2 inhibitor is increased by at least about 3-fold compared to a therapeutic including a BRAF axis inhibitor without a COX-2 inhibitor. In embodiments, anti-cancer response to a therapeutic including a BRAF axis inhibitor and a COX-2 inhibitor is increased by at least about 4-fold compared to a therapeutic including a BRAF axis inhibitor without a COX-2 inhibitor. In embodiments, anti-cancer response to a therapeutic including a BRAF axis inhibitor and a COX-2 inhibitor is increased by at least about 5-fold compared to a therapeutic including a BRAF axis inhibitor without a COX-2 inhibitor. In embodiments, anti-cancer response to a therapeutic including a BRAF axis inhibitor and a COX-2 inhibitor is increased by at least about 6-fold compared to a therapeutic including a BRAF axis inhibitor without a COX-2 inhibitor. In embodiments, anti-cancer response to a therapeutic including a BRAF axis inhibitor and a COX-2 inhibitor is increased by at least about 7-fold compared to a therapeutic including a BRAF axis inhibitor without a COX-2 inhibitor. In embodiments, anti-cancer response to a therapeutic including a BRAF axis inhibitor and a COX-2 inhibitor is increased by at least about 8-fold compared to a therapeutic including a BRAF axis inhibitor without a COX-2 inhibitor. In embodiments, anti-cancer response to a therapeutic including a BRAF axis inhibitor and a COX-2 inhibitor is increased by at least about 9-fold compared to a therapeutic including a BRAF axis inhibitor without a COX-2 inhibitor. In embodiments, anti-cancer response to a therapeutic including a BRAF axis inhibitor and a COX-2 inhibitor is increased by at least about 10-fold compared to a therapeutic including a BRAF axis inhibitor without a COX-2 inhibitor. In embodiments, anti-cancer response to a therapeutic including a BRAF axis inhibitor and a COX-2 inhibitor is increased by at least about 15-fold compared to a therapeutic including a BRAF axis inhibitor without a COX-2 inhibitor. In embodiments, anti-cancer response to a therapeutic including a BRAF axis inhibitor and a COX-2 inhibitor is increased by at least about 20-fold compared to a therapeutic including a BRAF axis inhibitor without a COX-2 inhibitor. In embodiments, anti-cancer response to a therapeutic including a BRAF axis inhibitor and a COX-2 inhibitor is increased by at least about 25-fold compared to a therapeutic including a BRAF axis inhibitor without a COX-2 inhibitor. In embodiments, anti-cancer response to a therapeutic including a BRAF axis inhibitor and a COX-2 inhibitor is increased by at least about 30-fold compared to a therapeutic including a BRAF axis inhibitor without a COX-2 inhibitor. In embodiments, anti-cancer response to a therapeutic including a BRAF axis inhibitor and a COX-2 inhibitor is increased by at least about 35-fold compared to a therapeutic including a BRAF axis inhibitor without a COX-2 inhibitor. In embodiments, anti-cancer response to a therapeutic including a BRAF axis inhibitor and a COX-2 inhibitor is increased by at least about 40-fold compared to a therapeutic including a BRAF axis inhibitor without a COX-2 inhibitor. In embodiments, anti-cancer response to a therapeutic including a BRAF axis inhibitor and a COX-2 inhibitor is increased by at least about 45-fold compared to a therapeutic including a BRAF axis inhibitor without a COX-2 inhibitor. In embodiments, anti-cancer response to a therapeutic including a BRAF axis inhibitor and a COX-2 inhibitor is increased by at least about 50-fold compared to a therapeutic including a BRAF axis inhibitor without a COX-2 inhibitor. In embodiments, anti-cancer response to a therapeutic including a BRAF axis inhibitor and a COX-2 inhibitor is increased by at least about 100-fold compared to a therapeutic including a BRAF axis inhibitor without a COX-2 inhibitor. In embodiments, anti-cancer response to a therapeutic including a BRAF axis inhibitor and a COX-2 inhibitor is increased by at least about 150-fold compared to a therapeutic including a BRAF axis inhibitor without a COX-2 inhibitor. In embodiments, anti-cancer response to a therapeutic including a BRAF axis inhibitor and a COX-2 inhibitor is increased by at least about 200-fold compared to a therapeutic including a BRAF axis inhibitor without a COX-2 inhibitor.

Thus, in an aspect is provided a method of treating a serine/threonine-protein kinase B-Raf (BRAF) intrinsically resistant cancer in a subject, including administering to the subject a combined effective amount of a BRAF axis inhibitor and a cyclooxygenase-2 (COX-2) inhibitor. In embodiments, the BRAF axis inhibitor includes a BRAF inhibitor, a MEK inhibitor, an ERK inhibitor, or a combination thereof. In embodiments, the BRAF axis inhibitor includes a BRAF inhibitor. In embodiments, the BRAF axis inhibitor includes a MEK inhibitor. In embodiments, the BRAF axis inhibitor includes an ERK inhibitor. In embodiments, the BRAF axis inhibitor includes a plurality of BRAF axis inhibitors. In embodiments, the BRAF axis inhibitor includes a BRAF inhibitor and an MEK inhibitor. In embodiments, the BRAF axis inhibitor includes a BRAF inhibitor and an ERK inhibitor. In embodiments, the BRAF axis inhibitor includes a MEK inhibitor and an ERK inhibitor. In embodiments, the BRAF axis inhibitor includes a BRAF inhibitor, a MEK inhibitor, and an ERK inhibitor. In embodiments, the method includes administering to a subject a BRAF axis inhibitor as provided herein and a COX-2 inhibitor as provided herein and no other anti-cancer agent.

For the methods provided herein, in embodiments, the MEK inhibitor specifically targets MEK. For example, the MEK inhibitor may specifically bind the active site of MEK. In embodiments, the MEK inhibitor specifically targets MEK and no other kinase. In embodiments, the MEK inhibitor targets MEK and one or more additional kinases (e.g. a multi-kinase inhibitor). In embodiments, the MEK inhibitor is selumetinib, mirdametinib, trametinib, U0126-EtOH, PD184352, PD98059, BIX 02189, pimasertib, pelitinib, BIX 02188, TAK-733, AZD8330, binimetinib, SL-327, refametinib, zapnometinib, GDC-0623, BI-847325, R05126766, cobimetinib, PD318088, honokiol, APS-2-79 HCl, myricetin, or a combination thereof. In embodiments, the MEK inhibitor is trametinib, binimetinib, cobimetinib, selumetinib, or a combination thereof. In embodiments, the MEK inhibitor is mirdametinib. In embodiments, the MEK inhibitor is U0126-EtOH. In embodiments, the MEK inhibitor is PD184352. In embodiments, the MEK inhibitor is PD98059. In embodiments, the MEK inhibitor is BIX 02189. In embodiments, the MEK inhibitor is pimasertib. In embodiments, the MEK inhibitor is pelitinib. In embodiments, the MEK inhibitor is BIX 02188. In embodiments, the MEK inhibitor is TAK-733. In embodiments, the MEK inhibitor is AZD8330. In embodiments, the MEK inhibitor is SL-327. In embodiments, the MEK inhibitor is refametinib. In embodiments, the MEK inhibitor is zapnometinib. In embodiments, the MEK inhibitor is GDC-0623. In embodiments, the MEK inhibitor is BI-847325. In embodiments, the MEK inhibitor is R05126766. In embodiments, the MEK inhibitor is PD318088. In embodiments, the MEK inhibitor is honokiol. In embodiments, the MEK inhibitor is APS-2-79 HCl. In embodiments, the MEK inhibitor is myricetin. In embodiments, the MEK inhibitor is trametinib. In embodiments, the MEK inhibitor is binimetinib. In embodiments, the MEK inhibitor is cobimetinib. In embodiments, the MEK inhibitor is selumetinib.

For the methods provided herein, in embodiments, the ERK inhibitor specifically targets ERK. For example, the ERK inhibitor may specifically bind the active site of ERK. In embodiments, the ERK inhibitor specifically targets ERK and no other kinase. In embodiments, the ERK inhibitor targets ERK and one or more additional kinases (e.g. a multi-kinase inhibitor). In embodiments, the ERK inhibitor is ulixertinib (BVD-523), ravoxertinib (GDC-0994), LY3214996, LTT462, or a combination thereof. In embodiments, the ERK inhibitor is ulixertinib (BVD-523). In embodiments, the ERK inhibitor is ravoxertinib (GDC-0994). In embodiments, the ERK inhibitor is LY3214996. In embodiments, the ERK inhibitor is LTT462.

For the methods provided herein, in embodiments, the BRAF inhibitor specifically targets BRAF. For example, the BRAF inhibitor may specifically bind the active site of BRAF. In embodiments, the BRAF inhibitor specifically binds BRAF^{V600E}. For example, the BRAF inhibitor may bind the ATP-binding site of BRAF^{V600E}. In embodiments, the BRAF inhibitor targets BRAF and one or more additional kinases (e.g. a multi-kinase inhibitor). In embodiments, the BRAF inhibitor is encorafenib, vemurafenib, or dabrafenib, sorafenib, regorafenib, LY3009120, or a combination thereof. In embodiments, the BRAF inhibitor is encorafenib, vemurafenib, or dabrafenib, or a combination thereof. In embodiments, the BRAF inhibitor is Sorafenib, Regorafenib, LY3009120, or a combination thereof. In embodiments, the BRAF inhibitor is encorafenib. In embodiments, the BRAF inhibitor is vemurafenib. In embodiments, the BRAF inhibitor is dabrafenib. In embodiments, the BRAF inhibitor is Sorafenib. In embodiments, the BRAF inhibitor is Regorafenib. In embodiments, the BRAF inhibitor is LY3009120.

In embodiments, the BRAF axis inhibitor and the COX-2 inhibitor are administered simultaneously. In embodiments, the BRAF axis inhibitor and the COX-2 inhibitor are administered sequentially.

As described throughout the specification, combination therapies including an BRAF axis inhibitor, an EGFR inhibitor, and a COX-2 inhibitor is effective for the treatment of BRAF intrinsically resistant cancer. Applicant found that the addition of a COX-2 inhibitor to combination therapies including a BRAF axis inhibitor and an EGFR inhibitor increases or potentiates the anti-cancer response to the therapy. In embodiments, the anti-cancer response to a therapeutic including a BRAF axis inhibitor, EGFR inhibitor, and a COX-2 inhibitor is increased by at least about 40%, 50%, 60%, 70%, 80%, 90%, or 100% compared to a therapeutic including a BRAF axis inhibitor and an EGFR inhibitor without a COX-2 inhibitor. In embodiments, anti-cancer response to a therapeutic including a BRAF axis inhibitor, EGFR inhibitor, and a COX-2 inhibitor is increased by at least about 40% compared to a therapeutic including a BRAF axis inhibitor and an EGFR inhibitor without a COX-2 inhibitor. In embodiments, anti-cancer response to a therapeutic including a BRAF axis inhibitor, EGFR inhibitor, and a COX-2 inhibitor is increased by at least about 50% compared to a therapeutic including a BRAF axis inhibitor and an EGFR inhibitor without a COX-2 inhibitor. In embodiments, anti-cancer response to a therapeutic including a BRAF axis inhibitor, EGFR inhibitor, and a COX-2 inhibitor is increased by at least about 60% compared to a therapeutic including a BRAF axis inhibitor and an EGFR inhibitor without a COX-2 inhibitor. In embodiments, anti-cancer response to a therapeutic including a BRAF axis inhibitor, EGFR inhibitor, and a COX-2 inhibitor is increased by at least about 70% compared to a therapeutic including a BRAF axis inhibitor and an EGFR inhibitor without a COX-2 inhibitor. In embodiments, anti-cancer response to a therapeutic including a BRAF axis inhibitor, EGFR inhibitor, and a COX-2 inhibitor is increased by at least about 80% compared to a therapeutic including a BRAF axis inhibitor and an EGFR inhibitor without a COX-2 inhibitor. In embodiments, anti-cancer response to a therapeutic including a BRAF axis inhibitor, EGFR inhibitor, and a COX-2 inhibitor is increased by at least about 85% compared to a therapeutic including a BRAF axis inhibitor and an EGFR inhibitor without a COX-2 inhibitor. In embodiments, anti-cancer response to a therapeutic including a BRAF axis inhibitor, EGFR inhibitor, and a COX-2 inhibitor is increased by at least about 90% compared to a therapeutic including a BRAF axis inhibitor and an EGFR inhibitor without a COX-2 inhibitor. In embodiments, anti-cancer response to a therapeutic including a BRAF axis inhibitor, EGFR inhibitor, and a COX-2 inhibitor is increased by at least about 91% compared to a therapeutic including a BRAF axis inhibitor and an EGFR inhibitor without a COX-2 inhibitor. In embodiments, anti-cancer response to a therapeutic including a BRAF axis inhibitor, EGFR inhibitor, and a COX-2 inhibitor is increased by at least about 92% compared to a therapeutic including a BRAF axis inhibitor and an EGFR inhibitor without a COX-2 inhibitor. In embodiments, anti-cancer response to a therapeutic including a BRAF axis inhibitor, EGFR inhibitor, and a COX-2 inhibitor is increased by at least about 93% compared to a therapeutic including a BRAF axis inhibitor and an EGFR inhibitor without a COX-2 inhibitor. In embodiments, anti-cancer response to a therapeutic including a BRAF axis inhibitor, EGFR inhibitor, and a COX-2 inhibitor is increased by at least about 94% compared to a therapeutic including a BRAF axis inhibitor and an EGFR inhibitor without a COX-2 inhibitor. In embodiments, anti-cancer response to a therapeutic including a BRAF axis inhibitor, EGFR inhibitor, and a COX-2 inhibitor is increased by at least about 95% compared to a therapeutic including a BRAF axis inhibitor and an EGFR inhibitor without a COX-2 inhibitor. In embodiments, anti-cancer response to a therapeutic including a BRAF axis inhibitor, EGFR inhibitor, and a COX-2 inhibitor is increased by at least about 96% compared to a therapeutic including a BRAF axis inhibitor and an EGFR inhibitor without a COX-2 inhibitor. In embodiments, anti-cancer response to a therapeutic including a BRAF axis inhibitor, EGFR inhibitor, and a COX-2 inhibitor is increased by at least about 97% compared to a therapeutic including a BRAF axis inhibitor and an EGFR inhibitor without a COX-2 inhibitor. In embodiments, anti-cancer response to a therapeutic including a BRAF axis inhibitor, EGFR inhibitor, and a COX-2 inhibitor is increased by at least about 98% compared to a therapeutic including a BRAF axis inhibitor and an EGFR inhibitor without a COX-2 inhibitor. In embodiments, anti-cancer response to a therapeutic including a BRAF axis inhibitor, EGFR inhibitor, and a COX-2 inhibitor is increased by at least about 99% compared to a therapeutic including a BRAF axis inhibitor and an EGFR inhibitor without a COX-2 inhibitor. In embodiments, anti-cancer response to a therapeutic including a BRAF axis inhibitor, EGFR inhibitor, and a COX-2 inhibitor is increased by at least about 100% compared to a therapeutic including a BRAF axis inhibitor and an EGFR inhibitor without a COX-2 inhibitor.

In embodiments, the anti-cancer response to a therapeutic including a BRAF inhibitor, EGFR inhibitor, and COX-2 inhibitor is increased by at least about 0.2-fold, 0.4-fold, 0.6-fold, 0.8-fold, 1-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 100-fold, 150-fold, or 200-fold compared to a therapeutic including a BRAF inhibitor and EGFR inhibitor without a COX-2 inhibitor. In embodiments, the anti-cancer response to a therapeutic including a BRAF inhibitor, EGFR inhibitor, and COX-2 inhibitor is increased by at least about 0.1-fold compared to a therapeutic including a BRAF inhibitor and EGFR inhibitor without a COX-2 inhibitor. In embodiments, the anti-cancer response to a therapeutic including a BRAF inhibitor, EGFR inhibitor, and COX-2 inhibitor is increased by at least about 0.2-fold compared to a therapeutic including a BRAF inhibitor and EGFR inhibitor without a COX-2 inhibitor. In embodiments, the anti-cancer response to a therapeutic including a BRAF inhibitor, EGFR inhibitor, and COX-2 inhibitor is increased by at least about 0.4-fold compared to a therapeutic including a BRAF inhibitor and EGFR inhibitor without a COX-2 inhibitor. In embodiments, the anti-cancer response to a therapeutic including a BRAF inhibitor, EGFR inhibitor, and COX-2 inhibitor is increased by at least about 0.6-fold compared to a therapeutic including a BRAF inhibitor and EGFR inhibitor without a COX-2 inhibitor. In embodiments, the anti-cancer response to a therapeutic including a BRAF inhibitor, EGFR inhibitor, and COX-2 inhibitor is increased by at least about 0.8-fold compared to a therapeutic including a BRAF inhibitor and EGFR inhibitor without a COX-2 inhibitor. In embodiments, the anti-cancer response to a therapeutic including a BRAF inhibitor, EGFR inhibitor, and COX-2 inhibitor is increased by at least about 1-fold compared to a therapeutic including a BRAF inhibitor and EGFR inhibitor without a COX-2 inhibitor. In embodiments, the anti-cancer response to a therapeutic including a BRAF inhibitor, EGFR inhibitor, and COX-2 inhibitor is increased by at least about 2-fold compared to a therapeutic including a BRAF inhibitor and EGFR inhibitor without a COX-2 inhibitor. In embodiments, the anti-cancer response to a therapeutic including a BRAF inhibitor, EGFR inhibitor, and COX-2 inhibitor is increased by at least about 3-fold compared to a therapeutic including a BRAF inhibitor and EGFR inhibitor without a COX-2 inhibitor. In embodiments, the anti-cancer response to a therapeutic including a BRAF inhibitor, EGFR inhibitor, and COX-2 inhibitor is increased by at least about 4-fold compared to a therapeutic including a BRAF inhibitor and EGFR inhibitor without a COX-2 inhibitor. In embodiments, the anti-cancer response to a therapeutic including a BRAF inhibitor, EGFR inhibitor, and COX-2 inhibitor is increased by at least about 5-fold compared to a therapeutic including a BRAF inhibitor and EGFR inhibitor without a COX-2 inhibitor. In embodiments, the anti-cancer response to a therapeutic including a BRAF inhibitor, EGFR inhibitor, and COX-2 inhibitor is increased by at least about 6-fold compared to a therapeutic including a BRAF inhibitor and EGFR inhibitor without a COX-2 inhibitor. In embodiments, the anti-cancer response to a therapeutic including a BRAF inhibitor, EGFR inhibitor, and COX-2 inhibitor is increased by at least about 7-fold compared to a therapeutic including a BRAF inhibitor and EGFR inhibitor without a COX-2 inhibitor. In embodiments, the anti-cancer response to a therapeutic including a BRAF inhibitor, EGFR inhibitor, and COX-2 inhibitor is increased by at least about 8-fold compared to a therapeutic including a BRAF inhibitor and EGFR inhibitor without a COX-2 inhibitor. In embodiments, the anti-cancer response to a therapeutic including a BRAF inhibitor, EGFR inhibitor, and COX-2 inhibitor is increased by at least about 9-fold compared to a therapeutic including a BRAF inhibitor and EGFR inhibitor without a COX-2 inhibitor. In embodiments, the anti-cancer response to a therapeutic including a BRAF inhibitor, EGFR inhibitor, and COX-2 inhibitor is increased by at least about 10-fold compared to a therapeutic including a BRAF inhibitor and EGFR inhibitor without a COX-2 inhibitor. In embodiments, the anti-cancer response to a therapeutic including a BRAF inhibitor, EGFR inhibitor, and COX-2 inhibitor is increased by at least about 15-fold compared to a therapeutic including a BRAF inhibitor and EGFR inhibitor without a COX-2 inhibitor. In embodiments, the anti-cancer response to a therapeutic including a BRAF inhibitor, EGFR inhibitor, and COX-2 inhibitor is increased by at least about 20-fold compared to a therapeutic including a BRAF inhibitor and EGFR inhibitor without a COX-2 inhibitor. In embodiments, the anti-cancer response to a therapeutic including a BRAF inhibitor, EGFR inhibitor, and COX-2 inhibitor is increased by at least about 25-fold compared to a therapeutic including a BRAF inhibitor and EGFR inhibitor without a COX-2 inhibitor. In embodiments, the anti-cancer response to a therapeutic including a BRAF inhibitor, EGFR inhibitor, and COX-2 inhibitor is increased by at least about 30-fold compared to a therapeutic including a BRAF inhibitor and EGFR inhibitor without a COX-2 inhibitor. In embodiments, the anti-cancer response to a therapeutic including a BRAF inhibitor, EGFR inhibitor, and COX-2 inhibitor is increased by at least about 35-fold compared to a therapeutic including a BRAF inhibitor and EGFR inhibitor without a COX-2 inhibitor. In embodiments, the anti-cancer response to a therapeutic including a BRAF inhibitor, EGFR inhibitor, and COX-2 inhibitor is increased by at least about 40-fold compared to a therapeutic including a BRAF inhibitor and EGFR inhibitor without a COX-2 inhibitor. In embodiments, the anti-cancer response to a therapeutic including a BRAF inhibitor, EGFR inhibitor, and COX-2 inhibitor is increased by at least about 45-fold compared to a therapeutic including a BRAF inhibitor and EGFR inhibitor without a COX-2 inhibitor. In embodiments, the anti-cancer response to a therapeutic including a BRAF inhibitor, EGFR inhibitor, and COX-2 inhibitor is increased by at least about 50-fold compared to a therapeutic including a BRAF inhibitor and EGFR inhibitor without a COX-2 inhibitor. In embodiments, the anti-cancer response to a therapeutic including a BRAF inhibitor, EGFR inhibitor, and COX-2 inhibitor is increased by at least about 100-fold compared to a therapeutic including a BRAF inhibitor and EGFR inhibitor without a COX-2 inhibitor. In embodiments, the anti-cancer response to a therapeutic including a BRAF inhibitor, EGFR inhibitor, and COX-2 inhibitor is increased by at least about 150-fold compared to a therapeutic including a BRAF inhibitor and EGFR inhibitor without a COX-2 inhibitor. In embodiments, the anti-cancer response to a therapeutic including a BRAF inhibitor, EGFR inhibitor, and COX-2 inhibitor is increased by at least about 200-fold compared to a therapeutic including a BRAF inhibitor and EGFR inhibitor without a COX-2 inhibitor.

In embodiments, the method further includes administering to the subject an epidermal growth factor receptor (EGFR) inhibitor. In embodiments, the EGFR inhibitor is panitumumab, cetuximab, gefitinib, erlotinib, neratinib, lapatinib, necitumumab, osimertinib, dacomitinib, mobocertinib, vandetanib, or a combination thereof. In embodiments, the EGFR inhibitor is panitumumab, cetuximab, gefitinib, erlotinib, neratinib, lapatinib, necitumumab, or a combination thereof. In embodiments, the EGFR inhibitor is panitumumab. In embodiments, the EGFR inhibitor is cetuximab. In embodiments, the EGFR inhibitor is gefitinib. In embodiments, the EGFR inhibitor is erlotinib. In embodiments, the EGFR inhibitor is neratinib. In embodiments, the EGFR inhibitor is lapatinib. In embodiments, the EGFR inhibitor is necitumumab. In embodiments, the EGFR inhibitor is osimertinib. In embodiments, the EGFR inhibitor is dacomitinib. In embodiments, the EGFR inhibitor is mobocertinib. In embodiments, the EGFR inhibitor is vandetanib. In embodiments, the method includes administering to a subject a BRAF axis inhibitor as provided herein, a COX-2 inhibitor as provided herein, and an EGFR inhibitor as provided herein, and no other anti-cancer agent.

For the methods provided herein, in embodiments, the COX-2 inhibitor a selective COX-2 inhibitor. In embodiments, the COX-2 inhibitor is a non-selective COX-2 inhibitor. In embodiments, the COX-2 inhibitor targets members of the COX enzyme family, including COX-2. In embodiments, the COX-2 inhibitor is acetylsalicylic acid, choline magnesium trisalicylate, diflunisal, salsalate, fenoprofen, flurbiprofen, ibuprofen, ketoprofen, naproxen, oxaprozin, diclofenac, indomethacin, sulindac, tolmetin, meloxicam, piroxicam, meclofenamate, nefenamic acid, nabumetone, etodalac, ketorolac, celecoxib, valdecoxib, rofecoxib, or a combination thereof. In embodiments, the COX-2 inhibitor is celecoxib, valdecoxib, ketorolac, rofecoxib, etoricoxib, lumiracoxib, or a combination thereof. In embodiments, the COX-2 inhibitor is celecoxib, valdecoxib, ketorolac, rofecoxib, or a combination thereof. In embodiments, the COX-2 inhibitor is acetylsalicylic acid. In embodiments, the COX-2 inhibitor is choline magnesium trisalicylate. In embodiments, the COX-2 inhibitor is diflunisal. In embodiments, the COX-2 inhibitor is salsalate. In embodiments, the COX-2 inhibitor is fenoprofen. In embodiments, the COX-2 inhibitor is flurbiprofen. In embodiments, the COX-2 inhibitor is ketoprofen. In embodiments, the COX-2 inhibitor is naproxen. In embodiments, the COX-2 inhibitor is oxaprozin. In embodiments, the COX-2 inhibitor is diclofenac. In embodiments, the COX-2 inhibitor is indomethacin. In embodiments, the COX-2 inhibitor is sulindac. In embodiments, the COX-2 inhibitor is tolmetin. In embodiments, the COX-2 inhibitor is meloxicam. In embodiments, the COX-2 inhibitor is piroxicam. In embodiments, the COX-2 inhibitor is meclofenamate. In embodiments, the COX-2 inhibitor is nefenamic acid. In embodiments, the COX-2 inhibitor is nabumetone. In embodiments, the COX-2 inhibitor is etoricoxib. In embodiments, the COX-2 inhibitor is lumiracoxib. In embodiments, the COX-2 inhibitor is celecoxib. In embodiments, the COX-2 inhibitor is valdecoxib. In embodiments, the COX-2 inhibitor is ketorolac. In embodiments, the COX-2 inhibitor is rofecoxib.

Applicant demonstrates herein that COX-2 inhibitors have a synergistic effect on BRAF axis inhibitors. As used herein, the terms “synergy”, “synergism”, “synergistic”, “combined synergistic amount”, and “synergistic therapeutic effect” which are used herein interchangeably, refer to a measured effect of the compound administered in combination where the measured effect is greater than the sum of the individual effects of each of the compounds provided herein administered alone as a single agent. In embodiments, the methods provided herein include administering a BRAF axis inhibitor and a COX-2 inhibitor in a combined synergistic amount.

In embodiments, the synergistic effect is cancer cell death. Cancer cell death can be quantified, for example, by a decrease in tumor volume or decrease in the number of cancer cells. In embodiments, synergy between the BRAF axis inhibitor and COX-2 inhibitor results in at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% greater decrease in tumor volume or number of cancer cells than the sum of the decrease when the BRAF axis inhibitor and COX-2 inhibitor are used individually and separately.

In instances, the synergistic effect is inhibition of metastasis of cancer in a subject. In embodiments, synergy between the BRAF axis inhibitor and COX-2 inhibitor results in at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% greater inhibition of metastasis of a cancer in a subject than the sum of the inhibition when the BRAF axis inhibitor and COX-2 inhibitor are used individually and separately.

In embodiments, a synergistic amount may be about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of the amount of the BRAF axis inhibitor provided herein when used separately from the COX-2 inhibitor. In embodiments, a synergistic amount may be about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of the amount of the COX-2 inhibitor when used separately from the BRAF axis inhibitor.

The synergistic effect may be a cancer-treating effect such as a colorectal cancer (i.e. a colorectal cancer-treating synergistic effect), thyroid cancer (i.e. a thyroid cancer-treating synergistic effect), glioblastoma (i.e. a glioblastoma-treating synergistic effect), sarcoma (i.e. a sarcoma-treating synergistic effect), and hepto-sarcoma (i.e. a hepto-sarcoma treating synergistic effect.

In embodiments, the method includes administering to the subject a BRAF inhibitor, a MEK inhibitor, and a COX-2 inhibitor, wherein the BRAF inhibitor is dabrafenib, the MEK inhibitor is trametinib, and the COX-2 inhibitor is celecoxib. In embodiments, the method includes administering to the subject a BRAF inhibitor, a MEK inhibitor, and a COX-2 inhibitor, wherein the BRAF inhibitor is vemurafenib, the MEK inhibitor is trametinib, and the COX-2 inhibitor is celecoxib.

In embodiments, the method includes administering to the subject a MEK inhibitor, an EGFR inhibitor, and a COX-2 inhibitor, wherein the MEK inhibitor is trametinib, the EGFR inhibitor is gefitinib, and the COX-2 inhibitor is celecoxib.

In embodiments, the method includes administering to the subject a BRAF inhibitor, a MEK inhibitor, an EGFR inhibitor, and a COX-2 inhibitor, wherein the BRAF inhibitor is dabrafenib, the MEK inhibitor is trametinib, the EGFR inhibitor is panitumumab, and the COX-2 inhibitor is celecoxib. In embodiments, the method includes administering to the subject a BRAF inhibitor, a MEK inhibitor, an EGFR inhibitor, and a COX-2 inhibitor, wherein the BRAF inhibitor is vemurafenib, the MEK inhibitor is trametinib, the EGFR inhibitor is gefitinib, and the COX-2 inhibitor is celecoxib.

As described throughout the specification, Applicant discovered that SRC may be upregulated or activated in BRAF intrinsically resistant cancers (e.g. colorectal cancer) upon treatment with BRAF axis inhibitors optionally combined with EGFR inhibitors. Applicant found that activation of SRC induces upregulation of the activity of the transcription factor beta-catenin, which is involved in cancer cell adaptive responses. Applicant discovered that administration of a SRC inhibitor increases efficacy of combination treatments described herein including embodiments thereof. Thus, in embodiments, the method further includes administering to the subject a SRC inhibitor. In embodiments, the method includes administering to a subject a BRAF axis inhibitor as provided herein, a COX-2 inhibitor as provided herein, a SRC inhibitor as provided herein, and no other anti-cancer agent. In embodiments, the SRC inhibitor is dasatinib, saracatinib, ponatinib, bosutinib, pelitinib, resveratrol, KX2-391, NVP-BHG712, ENMD-2076, PP2, PP121, PP1, 2,3-Methylenedioxy-beta-nitrostyrene (MNS), 1-NM-PP1, NPK1-IN-2, XL228, DGY-06-116, Elzovanitinib, eCF506, RK 24466, 1 Naphtyl PP1 (1-NA-PP1), AMG-47a, Src Inhibitor 1, KX1-004, Myristic Acid, 7-Hydroxy-4-chromone, dasatinib hydrochloride, UM-164, repotrectinib, CCT196969, ON123300, SU6656, dasatinib monohydrate, WH-4-023, doramapimod, TPX-0046, dehydroabietic acid, ginkogolic acid C17:1, AD80, quercetin, or a combination thereof. In embodiments, the SRC inhibitor is dasatinib, saracatinib, bosutinib, ponatinib, imatinib, or a combination thereof. In embodiments, the SRC inhibitor is dasatinib. In embodiments, the SRC inhibitor is saracatinib. In embodiments, the SRC inhibitor is bosutinib. In embodiments, the SRC inhibitor is ponatinib. In embodiments, the SRC inhibitor is imatinib. In embodiments, the SRC inhibitor is pelitinib. In embodiments, the SRC inhibitor is resveratrol. In embodiments, the SRC inhibitor is KX2-391. In embodiments, the SRC inhibitor is NVP-BHG712. In embodiments, the SRC inhibitor is ENMD-2076. In embodiments, the SRC inhibitor is PP2. In embodiments, the SRC inhibitor is PP121. In embodiments, the SRC inhibitor is PP1. In embodiments, the SRC inhibitor is 2,3-Methylenedioxy-beta-nitrostyrene (MNS). In embodiments, the SRC inhibitor is 1-NM-PP. In embodiments, the SRC inhibitor is NPK1-IN-2. In embodiments, the SRC inhibitor is XL228. In embodiments, the SRC inhibitor is DGY-06-116. In embodiments, the SRC inhibitor is Elzovanitinib. In embodiments, the SRC inhibitor is eCF506. In embodiments, the SRC inhibitor is RK 24466. In embodiments, the SRC inhibitor is 1 Naphtyl PP1 (1-NA-PP1). In embodiments, the SRC inhibitor is AMG-47a. In embodiments, the SRC inhibitor is Src Inhibitor 1. In embodiments, the SRC inhibitor is KX1-004. In embodiments, the SRC inhibitor is Myristic Acid. In embodiments, the SRC inhibitor is 7-Hydroxy-4-chromone. In embodiments, the SRC inhibitor is dasatinib hydrochloride. In embodiments, the SRC inhibitor is UM-164. In embodiments, the SRC inhibitor is repotrectinib. In embodiments, the SRC inhibitor is CCT196969. In embodiments, the SRC inhibitor is ON123300. In embodiments, the SRC inhibitor is SU6656. In embodiments, the SRC inhibitor is dasatinib monohydrate. In embodiments, the SRC inhibitor is WH-4-023. In embodiments, the SRC inhibitor is doramapimod. In embodiments, the SRC inhibitor is TPX-0046. In embodiments, the SRC inhibitor is dehydroabietic acid. In embodiments, the SRC inhibitor is ginkogolic acid C17:1. In embodiments, the SRC inhibitor is AD80. In embodiments, the SRC inhibitor is quercetin.

In embodiments, the BRAF intrinsically resistant cancer is colorectal cancer, thyroid cancer, glioblastoma, sarcoma, or hepto-sarcoma. In embodiments, the BRAF intrinsically resistant cancer is colorectal cancer. In embodiments, the BRAF intrinsically resistant cancer is thyroid cancer. In embodiments, the BRAF intrinsically resistant cancer is glioblastoma. In embodiments, the BRAF intrinsically resistant cancer is sarcoma. In embodiments, the BRAF intrinsically resistant cancer is hepto-sarcoma. In embodiments, the BRAF intrinsically resistant cancer is not melanoma.

### Pharmaceutical Kits

Provided herein are, inter alia, compositions and methods of use for combination treatments including a BRAF axis inhbitor and a COX-2 inhibitor. In embodiments, the combination treatments further include an EGFR inhibitor. Without wishing to be bound by scientific theory, Applicant discovered that the combination treatments provided herein are surprising effective for treating BRAF intrinsically resistant cancers by, for example, inhibiting a cancer cell adaptive response (e.g. pro-survival mechanism) activated or up-regulated by BRAF axis inhibitors. The compositions and methods of use have been shown to effectively treat BRAF intrinsically resistant cancers (e.g. colorectal cancer), and further demonstrate low toxicity and side effects. Further, the combination treatments, including BRAF axis inhibitors and COX-2 inhibitors, are demonstrated to have a synergistic effect.

In an aspect is provided a pharmaceutical kit including: (i) a serine/threonine-protein kinase B-Raf (BRAF) axis inhibitor; and (ii) a and a cyclooxygenase-2 (COX-2) inhibitor. In embodiments, the kit includes a plurality of therapeutic agents of the combination therapy as a unit dosage form; e.g., the dosage form contains both a first and second therapeutic agent. Thus, in embodiments, the unit dosage form includes a BRAF axis inhibitor and a COX-2 inhibitor. In embodiments, the unit dosage form includes a BRAF axis inhibitor, a COX-2 inhibitor and no other active agents. In embodiments, the unit dosage form includes a BRAF axis inhibitor, a COX-2 inhibitor and no other therapeutic agent. In embodiments, the unit dosage form includes a BRAF axis inhibitor, a COX-2 inhibitor and no other anti-cancer agent. In embodiments, the kit includes discrete dosage forms (e.g., the first agent is contained in one dosage form and the second agent is contained in another dosage form). Thus, in embodiments, the BRAF axis inhibitor is in a first dosage form including the BRAF axis inhibitor. A “first dosage form” as provided herein refers to a discrete composition of a BRAF axis inhibitor and is separate from other dosage forms (e.g., the second dosage form including the COX-2 inhibitor). In embodiments, the first dosage form does not include any other active agents. In embodiments, the first dosage form does not include any other therapeutic agent. In embodiments, the first dosage form does not include any other anti-cancer agent. In embodiments, the COX-2 inhibitor is in a second dosage form including the COX-2 inhibitor. Likewise, a “second dosage form” as provided herein refers to a discrete composition of the COX-2 inhibitor and is separate from other dosage forms (e.g., the first dosage form of the BRAF axis inhibitor). In embodiments, the second dosage form does not include any other active agents. In embodiments, the first dosage form does not include any other therapeutic agent. In embodiments, the second dosage form does not include any other anti-cancer agent.

In embodiments, the unit dosage form includes more than one anti-cancer agent (e.g. a BRAF axis inhibitor and a COX-2 inhibitor), wherein each of the more than one one anti-cancer agents are different. In embodiments, each of the more than anti-cancer agent has a different target (e.g. targets BRAF and COX-2; targets different components of the BRAF axis (e.g. BRAF and MEK, BRAF and ERK, etc.)). In embodiments, the first or second dosage form may include multiple anti-cancer agents. In embodiments, the first dosage form includes more than one BRAF axis inhibitor, wherein the more than one BRAF axis inhibitor are different. In embodiments, the first dosage form includes a first BRAF axis inhibitor and a second BRAF axis inhibitor. In embodiments, the first dosage form includes a first BRAF axis inhibitor, a second BRAF axis inhibitor and a third BRAF axis inhibitor.

In embodiments, the unit dosage form includes a pharmaceutically acceptable excipient. In embodiments, the unit dosage form includes a plurality of therapeutic agents of the combination therapy (e.g. a BRAF axis inhibitor and a COX-2 inhibitor, etc.) and a pharmaceutically acceptable excipient. In embodiments, the unit dosage form may include a combination of two or more of a BRAF inhibitor, a MEK inhibitor, and an ERK inhibitor and a pharmaceutically acceptable excipient. In embodiments, the unit dosage form may include a combination of two or more of a BRAF axis inhibitor, a COX-2 inhibitor, an EGFR inhibitor, and a SRC inhibitor and a pharmaceutically acceptable excipient. In embodiments, the unit dosage form may include a combination of two or more of a BRAF inhibitor as provided herein and a pharmaceutically acceptable excipient. In embodiments, the unit dosage form may include a combination of two or more of a MEK inhibitor as provided herein and a pharmaceutically acceptable excipient. In embodiments, the unit dosage form may include a combination of two or more of a ERK inhibitor as provided herein and a pharmaceutically acceptable excipient. In embodiments, the unit dosage form may include a combination of two or more of a SRC inhibitor as provided herein and a pharmaceutically acceptable excipient. In embodiments, the unit dosage form may include a combination of two or more of a COX-2 axis inhibitor as provided herein and a pharmaceutically acceptable excipient.

In embodiments, the unit dosage form includes a single therapeutic agent (e.g. a BRAF axis inhibitor, a COX-2 inhibitor, etc.), and a pharmaceutically acceptable excipient. In embodiments, the unit dosage form includes a single therapeutic agent (e.g. a BRAF axis inhibitor, a COX-2 inhibitor, etc.) and no other therapeutic agents, and a pharmaceutically acceptable excipient. Thus, in embodiments, the unit dosage form includes a BRAF axis inhibitor and a pharmaceutically acceptable excipient. In embodiments, the unit dosage form includes a COX-2 inhibitor and a pharmaceutically acceptable excipient. In embodiments, the unit dosage form includes a SRC inhibitor and a pharmaceutically acceptable excipient. In embodiments, the unit dosage form includes an EGFR inhibitor and a pharmaceutically acceptable excipient. Thus, in embodiments, the pharmaceutical kit includes at least two unit dosage forms, each including a single therapeutic agent and a pharmaceutically acceptable excipient. In embodiments, the pharmaceutical kit includes a first unit dosage form including a therapeutic agent provided herein and a pharmaceutically acceptable excipient. In embodiments, the pharmaceutical kit includes a second unit dosage form including a therapeutic agent provided herein and a pharmaceutically acceptable excipient. In embodiments, the pharmaceutical kit includes a third unit dosage form including a therapeutic agent provided herein and a pharmaceutically acceptable excipient. In embodiments, the pharmaceutical kit includes a fourth unit dosage form including a therapeutic agent provided herein and a pharmaceutically acceptable excipient. In embodiments, the pharmaceutical kit includes a fifth unit dosage form including a therapeutic agent provided herein and a pharmaceutically acceptable excipient.

In embodiments, the BRAF axis inhibitor is a BRAF inhibitor, a MEK inhibitor, an ERK inhibitor, or a combination thereof. In embodiments, the BRAF axis inhibitor includes a BRAF inhibitor and a MEK inhibitor. In embodiments, the BRAF axis inhibitor includes a BRAF inhibitor and an ERK inhibitor. In embodiments, the BRAF axis inhibitor includes a MEK inhibitor and an ERK inhibitor. In embodiments, the BRAF axis inhibitor includes a BRAF inhibitor, a MEK inhibitor, and an ERK inhibitor.

In embodiments, the MEK inhibitor is selumetinib, mirdametinib, trametinib, U0126-EtOH, PD184352, PD98059, BIX 02189, pimasertib, pelitinib, BIX 02188, TAK-733, AZD8330, binimetinib, SL-327, refametinib, zapnometinib, GDC-0623, BI-847325, R05126766, cobimetinib, PD318088, honokiol, APS-2-79 HCl, myricetin, or a combination thereof. In embodiments, the MEK inhibitor is trametinib, binimetinib, cobimetinib, selumetinib, or a combination thereof. In embodiments, the MEK inhibitor is trametinib, binimetinib, or a combination thereof. In embodiments, the MEK inhibitor is trametinib.

In embodiments, the unit dosage form includes from about 0.1 mg to about 3 mg of trametinib. In embodiments, the unit dosage form includes from about 0.25 mg to about 3 mg of trametinib. In embodiments, the unit dosage form includes from about 0.5 mg to about 3 mg of trametinib. In embodiments, the unit dosage form includes from about 0.75 mg to about 3 mg of trametinib. In embodiments, the unit dosage form includes from about 1 mg to about 3 mg of trametinib. In embodiments, the unit dosage form includes from about 1.25 mg to about 3 mg of trametinib. In embodiments, the unit dosage form includes from about 1.5 mg to about 3 mg of trametinib. In embodiments, the unit dosage form includes from about 1.75 mg to about 3 mg of trametinib. In embodiments, the unit dosage form includes from about 2 mg to about 3 mg of trametinib. In embodiments, the unit dosage form includes from about 2.25 mg to about 3 mg of trametinib. In embodiments, the unit dosage form includes from about 2.5 mg to about 3 mg of trametinib. In embodiments, the unit dosage form includes from about 2.75 mg to about 3 mg of trametinib.

In embodiments, the unit dosage form includes from about 0.1 mg to about 2.75 mg of trametinib. In embodiments, the unit dosage form includes from about 0.1 mg to about 2.5 mg of trametinib. In embodiments, the unit dosage form includes from about 0.1 mg to about 2.25 mg of trametinib. In embodiments, the unit dosage form includes from about 0.1 mg to about 2 mg of trametinib. In embodiments, the unit dosage form includes from about 0.1 mg to about 1.75 mg of trametinib. In embodiments, the unit dosage form includes from about 0.1 mg to about 1.5 mg of trametinib. In embodiments, the unit dosage form includes from about 0.1 mg to about 1.25 mg of trametinib. In embodiments, the unit dosage form includes from about 0.1 mg to about 1 mg of trametinib. In embodiments, the unit dosage form includes from about 0.1 mg to about 0.75 mg of trametinib. In embodiments, the unit dosage form includes from about 0.1 mg to about 0.5 mg of trametinib. In embodiments, the unit dosage form includes from about 0.1 mg to about 0.25 mg of trametinib. In embodiments, the unit dosage form includes about 0.1 mg, 0.25 mg, 0.5 mg, 0.75 mg, 1 mg, 1.25 mg, 1.5 mg, 1.75 mg, 2 mg, 2.25 mg, 2.5 mg, 2.75 mg or 3 mg of trametinib. In embodiments, the unit dosage form includes about 0.5 mg of trametinib. In embodiments, the unit dosage form includes 0.5 mg of trametinib. In embodiments, the unit dosage form includes about 2 mg of trametinib. In embodiments, the unit dosage form includes 2 mg of trametinib.

In embodiments, the unit dosage form includes from about 1 mg to about 15 mg of binimetinib. In embodiments, the unit dosage form includes from about 5 mg to about 15 mg of binimetinib. In embodiments, the unit dosage form includes from about 10 mg to about 15 mg of binimetinib.

In embodiments, the unit dosage form includes from about 1 mg to about 10 mg of binimetinib. In embodiments, the unit dosage form includes from about 1 mg to about 5 mg of binimetinib. In embodiments, the unit dosage form includes from 1 mg to about 15 mg of binimetinib. In embodiments, the unit dosage form includes about 1 mg, 10 mg, or 15 mg of binimetinib.

In embodiments, the unit dosage form includes from about 5 to about 55 mg of binimetinib. In embodiments, the unit dosage form includes from about 10 to about 55 mg of binimetinib. In embodiments, the unit dosage form includes from about 15 to about 55 mg of binimetinib. In embodiments, the unit dosage form includes from about 20 to about 55 mg of binimetinib. In embodiments, the unit dosage form includes from about 25 to about 55 mg of binimetinib. In embodiments, the unit dosage form includes from about 30 to about 55 mg of binimetinib. In embodiments, the unit dosage form includes from about 35 to about 55 mg of binimetinib. In embodiments, the unit dosage form includes from about 40 to about 55 mg of binimetinib. In embodiments, the unit dosage form includes from about 45 to about 55 mg of binimetinib. In embodiments, the unit dosage form includes from about 50 to about 55 mg of binimetinib.

In embodiments, the unit dosage form includes from about 5 to about 50 mg of binimetinib. In embodiments, the unit dosage form includes from about 5 to about 45 mg of binimetinib. In embodiments, the unit dosage form includes from about 5 to about 40 mg of binimetinib. In embodiments, the unit dosage form includes from about 5 to about 35 mg of binimetinib. In embodiments, the unit dosage form includes from about 5 to about 30 mg of binimetinib. In embodiments, the unit dosage form includes from about 5 to about 25 mg of binimetinib. In embodiments, the unit dosage form includes from about 5 to about 20 mg of binimetinib. In embodiments, the unit dosage form includes from about 5 to about 15 mg of binimetinib. In embodiments, the unit dosage form includes from about 5 to about 10 mg of binimetinib. In embodiments, the unit dosage form includes about 5 mg, 10 mg, 15 mg, 20 mg, 25 mg, 30 mg, 35 mg, 40 mg, 45 mg, or 50 mg of binimetinib. In embodiments, the unit dosage form includes about 30 mg of binimetinib. In embodiments, the unit dosage form includes 30 mg of binimetinib. In embodiments, the unit dosage form includes about 45 mg of binimetinib. In embodiments, the unit dosage form includes 45 mg of binimetinib.

In embodiments, the unit dosage form includes from about 2 to about 30 mg of cobimetinib. In embodiments, the unit dosage form includes from about 4 to about 30 mg of cobimetinib. In embodiments, the unit dosage form includes from about 6 to about 30 mg of cobimetinib. In embodiments, the unit dosage form includes from about 8 to about 30 mg of cobimetinib. In embodiments, the unit dosage form includes from about 10 to about 30 mg of cobimetinib. In embodiments, the unit dosage form includes from about 12 to about 30 mg of cobimetinib. In embodiments, the unit dosage form includes from about 14 to about 30 mg of cobimetinib. In embodiments, the unit dosage form includes from about 16 to about 30 mg of cobimetinib. In embodiments, the unit dosage form includes from about 18 to about 30 mg of cobimetinib. In embodiments, the unit dosage form includes from about 20 to about 30 mg of cobimetinib. In embodiments, the unit dosage form includes from about 22 to about 30 mg of cobimetinib. In embodiments, the unit dosage form includes from about 24 to about 30 mg of cobimetinib. In embodiments, the unit dosage form includes from about 26 to about 30 mg of cobimetinib. In embodiments, the unit dosage form includes from about 28 to about 30 mg of cobimetinib.

In embodiments, the unit dosage form includes from about 2 to about 28 mg of cobimetinib. In embodiments, the unit dosage form includes from about 2 to about 26 mg of cobimetinib. In embodiments, the unit dosage form includes from about 2 to about 24 mg of cobimetinib. In embodiments, the unit dosage form includes from about 2 to about 22 mg of cobimetinib. In embodiments, the unit dosage form includes from about 2 to about 20 mg of cobimetinib. In embodiments, the unit dosage form includes from about 2 to about 18 mg of cobimetinib. In embodiments, the unit dosage form includes from about 2 to about 16 mg of cobimetinib. In embodiments, the unit dosage form includes from about 2 to about 14 mg of cobimetinib. In embodiments, the unit dosage form includes from about 2 to about 12 mg of cobimetinib. In embodiments, the unit dosage form includes from about 2 to about 10 mg of cobimetinib. In embodiments, the unit dosage form includes from about 2 to about 8 mg of cobimetinib. In embodiments, the unit dosage form includes from about 2 to about 6 mg of cobimetinib. In embodiments, the unit dosage form includes from about 2 to about 4 mg of cobimetinib. In embodiments, the unit dosage form includes about 2 mg, 4 mg, 6 mg, 8 mg, 10 mg, 12 mg, 14 mg, 16 mg, 18 mg, 20 mg, 22 mg, 24 mg, 26 mg, 28 mg, or 30 mg of cobimetinib. In embodiments, the unit dosage form includes about 20 mg of cobimetinib. In embodiments, the unit dosage form includes 20 mg of cobimetinib.

In embodiments, the unit dosage form includes from about 2 to about 30 mg of selumetinib. In embodiments, the unit dosage form includes from about 4 to about 30 mg of selumetinib. In embodiments, the unit dosage form includes from about 6 to about 30 mg of selumetinib. In embodiments, the unit dosage form includes from about 8 to about 30 mg of selumetinib. In embodiments, the unit dosage form includes from about 10 to about 30 mg of selumetinib. In embodiments, the unit dosage form includes from about 12 to about 30 mg of selumetinib. In embodiments, the unit dosage form includes from about 14 to about 30 mg of selumetinib. In embodiments, the unit dosage form includes from about 16 to about 30 mg of selumetinib. In embodiments, the unit dosage form includes from about 18 to about 30 mg of selumetinib. In embodiments, the unit dosage form includes from about 20 to about 30 mg of selumetinib. In embodiments, the unit dosage form includes from about 22 to about 30 mg of selumetinib. In embodiments, the unit dosage form includes from about 24 to about 30 mg of selumetinib. In embodiments, the unit dosage form includes from about 26 to about 30 mg of selumetinib. In embodiments, the unit dosage form includes from about 28 to about 30 mg of selumetinib.

In embodiments, the unit dosage form includes from about 2 to about 28 mg of selumetinib. In embodiments, the unit dosage form includes from about 2 to about 26 mg of selumetinib. In embodiments, the unit dosage form includes from about 2 to about 24 mg of selumetinib. In embodiments, the unit dosage form includes from about 2 to about 22 mg of selumetinib. In embodiments, the unit dosage form includes from about 2 to about 20 mg of selumetinib. In embodiments, the unit dosage form includes from about 2 to about 18 mg of selumetinib. In embodiments, the unit dosage form includes from about 2 to about 16 mg of selumetinib. In embodiments, the unit dosage form includes from about 2 to about 14 mg of selumetinib. In embodiments, the unit dosage form includes from about 2 to about 12 mg of selumetinib. In embodiments, the unit dosage form includes from about 2 to about 10 mg of selumetinib. In embodiments, the unit dosage form includes from about 2 to about 8 mg of selumetinib. In embodiments, the unit dosage form includes from about 2 to about 6 mg of selumetinib. In embodiments, the unit dosage form includes from about 2 to about 4 mg of selumetinib. In embodiments, the unit dosage form includes about 2 mg, 4 mg, 6 mg, 8 mg, 10 mg, 12 mg, 14 mg, 16 mg, 18 mg, 20 mg, 22 mg, 24 mg, 26 mg, 28 mg, or 30 mg of selumetinib. In embodiments, the unit dosage form includes about 10 mg of selumetinib. In embodiments, the unit dosage form includes 10 mg of selumetinib. In embodiments, the unit dosage form includes about 20 mg of selumetinib. In embodiments, the unit dosage form includes 20 mg of selumetinib.

In embodiments, the ERK inhibitor is ulixertinib (BVD-523), ravoxertinib (GDC-0994), LY3214996, LTT462, or a combination thereof.

In embodiments, the unit dosage form includes from about 200 mg to about 600 mg of ulixertinib. In embodiments, the unit dosage form includes from about 250 mg to about 600 mg of ulixertinib. In embodiments, the unit dosage form includes from about 300 mg to about 600 mg of ulixertinib. In embodiments, the unit dosage form includes from about 350 mg to about 600 mg of ulixertinib. In embodiments, the unit dosage form includes from about 400 mg to about 600 mg of ulixertinib. In embodiments, the unit dosage form includes from about 450 mg to about 600 mg of ulixertinib. In embodiments, the unit dosage form includes from about 500 mg to about 600 mg of ulixertinib. In embodiments, the unit dosage form includes from about 550 mg to about 600 mg of ulixertinib.

In embodiments, the unit dosage form includes from about 200 mg to about 550 mg of ulixertinib. In embodiments, the unit dosage form includes from about 200 mg to about 500 mg of ulixertinib. In embodiments, the unit dosage form includes from about 200 mg to about 450 mg of ulixertinib. In embodiments, the unit dosage form includes from about 200 mg to about 400 mg of ulixertinib. In embodiments, the unit dosage form includes from about 200 mg to about 350 mg of ulixertinib. In embodiments, the unit dosage form includes from about 200 mg to about 300 mg of ulixertinib. In embodiments, the unit dosage form includes from about 200 mg to about 250 mg of ulixertinib. In embodiments, the unit dosage form includes about 200 mg, 250 mg, 300 mg, 350 mg, 400 mg, 450 mg, 500 mg, 550 mg, or 600 mg of ulixertinib. In embodiments, the unit dosage form includes about 450 mg of ulixertinib. In embodiments, the unit dosage form includes 450 mg of ulixertinib.

In embodiments, the unit dosage form includes from about 50 mg to about 800 mg of ravoxertinib. In embodiments, the unit dosage form includes from about 100 mg to about 800 mg of ravoxertinib. In embodiments, the unit dosage form includes from about 150 mg to about 800 mg of ravoxertinib. In embodiments, the unit dosage form includes from about 200 mg to about 800 mg of ravoxertinib. In embodiments, the unit dosage form includes from about 250 mg to about 800 mg of ravoxertinib. In embodiments, the unit dosage form includes from about 300 mg to about 800 mg of ravoxertinib. In embodiments, the unit dosage form includes from about 350 mg to about 800 mg of ravoxertinib. In embodiments, the unit dosage form includes from about 400 mg to about 800 mg of ravoxertinib. In embodiments, the unit dosage form includes from about 450 mg to about 800 mg of ravoxertinib. In embodiments, the unit dosage form includes from about 500 mg to about 800 mg of ravoxertinib. In embodiments, the unit dosage form includes from about 550 mg to about 800 mg of ravoxertinib. In embodiments, the unit dosage form includes from about 600 mg to about 800 mg of ravoxertinib. In embodiments, the unit dosage form includes from about 650 mg to about 800 mg of ravoxertinib. In embodiments, the unit dosage form includes from about 700 mg to about 800 mg of ravoxertinib. In embodiments, the unit dosage form includes from about 750 mg to about 800 mg of ravoxertinib.

In embodiments, the unit dosage form includes from about 50 mg to about 750 mg of ravoxertinib. In embodiments, the unit dosage form includes from about 50 mg to about 700 mg of ravoxertinib. In embodiments, the unit dosage form includes from about 50 mg to about 650 mg of ravoxertinib. In embodiments, the unit dosage form includes from about 50 mg to about 600 mg of ravoxertinib. In embodiments, the unit dosage form includes from about 50 mg to about 550 mg of ravoxertinib. In embodiments, the unit dosage form includes from about 50 mg to about 500 mg of ravoxertinib. In embodiments, the unit dosage form includes from about 50 mg to about 450 mg of ravoxertinib. In embodiments, the unit dosage form includes from about 50 mg to about 400 mg of ravoxertinib. In embodiments, the unit dosage form includes from about 50 mg to about 350 mg of ravoxertinib. In embodiments, the unit dosage form includes from about 50 mg to about 300 mg of ravoxertinib. In embodiments, the unit dosage form includes from about 50 mg to about 250 mg of ravoxertinib. In embodiments, the unit dosage form includes from about 50 mg to about 200 mg of ravoxertinib. In embodiments, the unit dosage form includes from about 50 mg to about 150 mg of ravoxertinib. In embodiments, the unit dosage form includes from about 50 mg to about 100 mg of ravoxertinib. In embodiments, the unit dosage form includes about 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, or 800 mg of ravoxertinib.

In embodiments, the unit dosage form includes from about 100 mg to about 700 mg of LY3214996. In embodiments, the unit dosage form includes from about 150 mg to about 700 mg of LY3214996. In embodiments, the unit dosage form includes from about 200 mg to about 700 mg of LY3214996. In embodiments, the unit dosage form includes from about 250 mg to about 700 mg of LY3214996. In embodiments, the unit dosage form includes from about 300 mg to about 700 mg of LY3214996. In embodiments, the unit dosage form includes from about 350 mg to about 700 mg of LY3214996. In embodiments, the unit dosage form includes from about 400 mg to about 700 mg of LY3214996. In embodiments, the unit dosage form includes from about 450 mg to about 700 mg of LY3214996. In embodiments, the unit dosage form includes from about 500 mg to about 700 mg of LY3214996. In embodiments, the unit dosage form includes from about 550 mg to about 700 mg of LY3214996. In embodiments, the unit dosage form includes from about 600 mg to about 700 mg of LY3214996. In embodiments, the unit dosage form includes from about 650 mg to about 700 mg of LY3214996.

In embodiments, the unit dosage form includes from about 100 mg to about 650 mg of LY3214996. In embodiments, the unit dosage form includes from about 100 mg to about 600 mg of LY3214996. In embodiments, the unit dosage form includes from about 100 mg to about 550 mg of LY3214996. In embodiments, the unit dosage form includes from about 100 mg to about 500 mg of LY3214996. In embodiments, the unit dosage form includes from about 100 mg to about 450 mg of LY3214996. In embodiments, the unit dosage form includes from about 100 mg to about 400 mg of LY3214996. In embodiments, the unit dosage form includes from about 100 mg to about 350 mg of LY3214996. In embodiments, the unit dosage form includes from about 100 mg to about 300 mg of LY3214996. In embodiments, the unit dosage form includes from about 100 mg to about 250 mg of LY3214996. In embodiments, the unit dosage form includes from about 100 mg to about 200 mg of LY3214996. In embodiments, the unit dosage form includes from about 100 mg to about 150 mg of LY3214996. In embodiments, the unit dosage form includes about 100 mg, 150 mg, 200 mg, 250 mg, 300 mg, 350 mg, 400 mg, 450 mg, 500 mg, 550 mg, 600 mg, 650 mg, or 700 mg of LY3214996.

In embodiments, the unit dosage form includes from about 20 mg to about 700 mg of LTT462. In embodiments, the unit dosage form includes from about 50 mg to about 700 mg of LTT462. In embodiments, the unit dosage form includes from about 100 mg to about 700 mg of LTT462. In embodiments, the unit dosage form includes from about 150 mg to about 700 mg of LTT462. In embodiments, the unit dosage form includes from about 200 mg to about 700 mg of LTT462. In embodiments, the unit dosage form includes from about 250 mg to about 700 mg of LTT462. In embodiments, the unit dosage form includes from about 300 mg to about 700 mg of LTT462. In embodiments, the unit dosage form includes from about 350 mg to about 700 mg of LTT462. In embodiments, the unit dosage form includes from about 400 mg to about 700 mg of LTT462. In embodiments, the unit dosage form includes from about 450 mg to about 700 mg of LTT462. In embodiments, the unit dosage form includes from about 500 mg to about 700 mg of LTT462. In embodiments, the unit dosage form includes from about 550 mg to about 700 mg of LTT462. In embodiments, the unit dosage form includes from about 600 mg to about 700 mg of LTT462. In embodiments, the unit dosage form includes from about 650 mg to about 700 mg of LTT462.

In embodiments, the unit dosage form includes from about 20 mg to about 650 mg of LTT462. In embodiments, the unit dosage form includes from about 20 mg to about 600 mg of LTT462. In embodiments, the unit dosage form includes from about 20 mg to about 550 mg of LTT462. In embodiments, the unit dosage form includes from about 20 mg to about 500 mg of LTT462. In embodiments, the unit dosage form includes from about 20 mg to about 450 mg of LTT462. In embodiments, the unit dosage form includes from about 20 mg to about 400 mg of LTT462. In embodiments, the unit dosage form includes from about 20 mg to about 550 mg of LTT462. In embodiments, the unit dosage form includes from about 20 mg to about 500 mg of LTT462. In embodiments, the unit dosage form includes from about 20 mg to about 450 mg of LTT462. In embodiments, the unit dosage form includes from about 20 mg to about 400 mg of LTT462. In embodiments, the unit dosage form includes from about 20 mg to about 350 mg of LTT462. In embodiments, the unit dosage form includes from about 20 mg to about 300 mg of LTT462. In embodiments, the unit dosage form includes from about 20 mg to about 250 mg of LTT462. In embodiments, the unit dosage form includes from about 20 mg to about 200 mg of LTT462. In embodiments, the unit dosage form includes from about 20 mg to about 150 mg of LTT462. In embodiments, the unit dosage form includes from about 20 mg to about 100 mg of LTT462. In embodiments, the unit dosage form includes from about 20 mg to about 50 mg of LTT462. In embodiments, the unit dosage form includes from about 20 mg, 50 mg, 100 mg, 150 mg, 200 mg, 250 mg, 300 mg, 350 mg, 400 mg, 450 mg, 500 mg, 550 mg, 600 mg, 650 mg, or 700 mg of LTT462.

In embodiments, the BRAF inhibitor is encorafenib, vemurafenib, or dabrafenib, sorafenib, regorafenib, LY3009120, or a combination thereof. In embodiments, the BRAF inhibitor is encorafenib, vemurafenib, or dabrafenib, or a combination thereof. In embodiments, the BRAF inhibitor is vemurafenib.

In embodiments, the unit dosage form includes from about 20 mg to about 140 mg of encorafenib. In embodiments, the unit dosage form includes from about 30 mg to about 140 mg of encorafenib. In embodiments, the unit dosage form includes from about 40 mg to about 140 mg of encorafenib. In embodiments, the unit dosage form includes from about 50 mg to about 140 mg of encorafenib. In embodiments, the unit dosage form includes from about 60 mg to about 140 mg of encorafenib. In embodiments, the unit dosage form includes from about 70 mg to about 140 mg of encorafenib. In embodiments, the unit dosage form includes from about 80 mg to about 140 mg of encorafenib. In embodiments, the unit dosage form includes from about 90 mg to about 140 mg of encorafenib. In embodiments, the unit dosage form includes from about 100 mg to about 140 mg of encorafenib. In embodiments, the unit dosage form includes from about 110 mg to about 140 mg of encorafenib. In embodiments, the unit dosage form includes from about 120 mg to about 140 mg of encorafenib. In embodiments, the unit dosage form includes from about 130 mg to about 140 mg of encorafenib.

In embodiments, the unit dosage form includes from about 20 mg to about 130 mg of encorafenib. In embodiments, the unit dosage form includes from about 20 mg to about 120 mg of encorafenib. In embodiments, the unit dosage form includes from about 20 mg to about 110 mg of encorafenib. In embodiments, the unit dosage form includes from about 20 mg to about 100 mg of encorafenib. In embodiments, the unit dosage form includes from about 20 mg to about 90 mg of encorafenib. In embodiments, the unit dosage form includes from about 20 mg to about 80 mg of encorafenib. In embodiments, the unit dosage form includes from about 20 mg to about 70 mg of encorafenib. In embodiments, the unit dosage form includes from about 20 mg to about 60 mg of encorafenib. In embodiments, the unit dosage form includes from about 20 mg to about 50 mg of encorafenib. In embodiments, the unit dosage form includes from about 20 mg to about 40 mg of encorafenib. In embodiments, the unit dosage form includes from about 20 mg to about 30 mg of encorafenib. In embodiments, the unit dosage form includes about 75 mg of encorafenib. In embodiments, the unit dosage form includes 75 mg of encorafenib.

In embodiments, the unit dosage form includes from about 100 mg to about 300 mg of vemurafenib. In embodiments, the unit dosage form includes from about 120 mg to about 300 mg of vemurafenib. In embodiments, the unit dosage form includes from about 140 mg to about 300 mg of vemurafenib. In embodiments, the unit dosage form includes from about 160 mg to about 300 mg of vemurafenib. In embodiments, the unit dosage form includes from about 180 mg to about 300 mg of vemurafenib. In embodiments, the unit dosage form includes from about 200 mg to about 300 mg of vemurafenib. In embodiments, the unit dosage form includes from about 220 mg to about 300 mg of vemurafenib. In embodiments, the unit dosage form includes from about 240 mg to about 300 mg of vemurafenib. In embodiments, the unit dosage form includes from about 260 mg to about 300 mg of vemurafenib. In embodiments, the unit dosage form includes from about 280 mg to about 300 mg of vemurafenib.

In embodiments, the unit dosage form includes from about 100 mg to about 280 mg of vemurafenib. In embodiments, the unit dosage form includes from about 100 mg to about 260 mg of vemurafenib. In embodiments, the unit dosage form includes from about 100 mg to about 240 mg of vemurafenib. In embodiments, the unit dosage form includes from about 100 mg to about 220 mg of vemurafenib. In embodiments, the unit dosage form includes from about 100 mg to about 200 mg of vemurafenib. In embodiments, the unit dosage form includes from about 100 mg to about 180 mg of vemurafenib. In embodiments, the unit dosage form includes from about 100 mg to about 160 mg of vemurafenib. In embodiments, the unit dosage form includes from about 100 mg to about 140 mg of vemurafenib. In embodiments, the unit dosage form includes from about 100 mg to about 120 mg of vemurafenib. In embodiments, the unit dosage form includes about 100 mg, 120 mg, 140 mg, 160 mg, 180 mg, 200 mg, 220 mg, 240 mg, 260 mg, 280 mg, or 300 mg of vemurafenib. In embodiments, the unit dosage form includes about 240 mg of vemurafenib. In embodiments, the unit dosage form includes 240 mg of vemurafenib.

In embodiments, the unit dosage form includes from about 20 mg to about 120 mg of dabrafenib. In embodiments, the unit dosage form includes from about 30 mg to about 120 mg of dabrafenib. In embodiments, the unit dosage form includes from about 40 mg to about 120 mg of dabrafenib. In embodiments, the unit dosage form includes from about 50 mg to about 120 mg of dabrafenib. In embodiments, the unit dosage form includes from about 60 mg to about 120 mg of dabrafenib. In embodiments, the unit dosage form includes from about 70 mg to about 120 mg of dabrafenib. In embodiments, the unit dosage form includes from about 80 mg to about 120 mg of dabrafenib. In embodiments, the unit dosage form includes from about 90 mg to about 120 mg of dabrafenib. In embodiments, the unit dosage form includes from about 100 mg to about 120 mg of dabrafenib. In embodiments, the unit dosage form includes from about 110 mg to about 120 mg of dabrafenib.

In embodiments, the unit dosage form includes from about 20 mg to about 110 mg of dabrafenib. In embodiments, the unit dosage form includes from about 20 mg to about 100 mg of dabrafenib. In embodiments, the unit dosage form includes from about 20 mg to about 90 mg of dabrafenib. In embodiments, the unit dosage form includes from about 20 mg to about 80 mg of dabrafenib. In embodiments, the unit dosage form includes from about 20 mg to about 70 mg of dabrafenib. In embodiments, the unit dosage form includes from about 20 mg to about 60 mg of dabrafenib. In embodiments, the unit dosage form includes from about 20 mg to about 50 mg of dabrafenib. In embodiments, the unit dosage form includes from about 20 mg to about 40 mg of dabrafenib. In embodiments, the unit dosage form includes from about 20 mg to about 30 mg of dabrafenib. In embodiments, the unit dosage form includes about 20 mg, 30 mg, 40 mg, 50 mg, 60 mg, 70 mg, 80 mg, 90 mg, 100 mg, 110 mg, or 120 mg of dabrafenib. In embodiments, the unit dosage form includes from about 50 mg of dabrafenib. In embodiments, the unit dosage form includes 50 mg of dabrafenib. In embodiments, the unit dosage form includes about 75 mg of dabrafenib. In embodiments, the unit dosage form includes 75 mg of dabrafenib.

In embodiments, the COX-2 inhibitor is acetylsalicylic acid, choline magnesium trisalicylate, diflunisal, salsalate, fenoprofen, flurbiprofen, ibuprofen, ketoprofen, naproxen, oxaprozin, diclofenac, indomethacin, sulindac, tolmetin, meloxicam, piroxicam, meclofenamate, nefenamic acid, nabumetone, etodalac, ketorolac, celecoxib, valdecoxib, rofecoxib, or a combination thereof. In embodiments, the COX-2 inhibitor is celecoxib, valdecoxib, ketorolac, rofecoxib, etoricoxib, lumiracoxib, or a combination thereof. In embodiments, the COX-2 inhibitor is celecoxib, valdecoxib or a combination thereof. In embodiments, the COX-2 inhibitor is celecoxib.

In embodiments, the unit dosage form includes from about 20 mg to about 450 mg of celecoxib. In embodiments, the unit dosage form includes from about 50 mg to about 450 mg of celecoxib. In embodiments, the unit dosage form includes from about 100 mg to about 450 mg of celecoxib. In embodiments, the unit dosage form includes from about 150 mg to about 450 mg of celecoxib. In embodiments, the unit dosage form includes from about 200 mg to about 450 mg of celecoxib. In embodiments, the unit dosage form includes from about 250 mg to about 450 mg of celecoxib. In embodiments, the unit dosage form includes from about 300 mg to about 450 mg of celecoxib. In embodiments, the unit dosage form includes from about 350 mg to about 450 mg of celecoxib. In embodiments, the unit dosage form includes from about 400 mg to about 450 mg of celecoxib.

In embodiments, the unit dosage form includes from about 20 mg to about 400 mg of celecoxib. In embodiments, the unit dosage form includes from about 20 mg to about 350 mg of celecoxib. In embodiments, the unit dosage form includes from about 20 mg to about 300 mg of celecoxib. In embodiments, the unit dosage form includes from about 20 mg to about 250 mg of celecoxib. In embodiments, the unit dosage form includes from about 20 mg to about 200 mg of celecoxib. In embodiments, the unit dosage form includes from about 20 mg to about 150 mg of celecoxib. In embodiments, the unit dosage form includes from about 20 mg to about 100 mg of celecoxib. In embodiments, the unit dosage form includes from about 20 mg to about 50 mg of celecoxib. In embodiments, the unit dosage form includes about 20 mg, 50 mg, 100 mg, 150 mg, 200 mg, 250 mg, 300 mg, 350 mg, or 400 mg of celecoxib. In embodiments, the unit dosage form includes about 50 mg of celecoxib. In embodiments, the unit dosage form includes 50 mg of celecoxib. In embodiments, the unit dosage form includes about 100 mg of celecoxib. In embodiments, the unit dosage form includes 100 mg of celecoxib. In embodiments, the unit dosage form includes about 200 mg of celecoxib. In embodiments, the unit dosage form includes 200 mg of celecoxib. In embodiments, the unit dosage form includes about 400 mg of celecoxib. In embodiments, the unit dosage form includes 400 mg of celecoxib.

In embodiments, the unit dosage form includes from about 2 mg to about 30 mg of valdecoxib. In embodiments, the unit dosage form includes from about 4 mg to about 30 mg of valdecoxib. In embodiments, the unit dosage form includes from about 6 mg to about 30 mg of valdecoxib. In embodiments, the unit dosage form includes from about 8 mg to about 30 mg of valdecoxib. In embodiments, the unit dosage form includes from about 10 mg to about 30 mg of valdecoxib. In embodiments, the unit dosage form includes from about 12 mg to about 30 mg of valdecoxib. In embodiments, the unit dosage form includes from about 14 mg to about 30 mg of valdecoxib. In embodiments, the unit dosage form includes from about 18 mg to about 30 mg of valdecoxib. In embodiments, the unit dosage form includes from about 20 mg to about 30 mg of valdecoxib. In embodiments, the unit dosage form includes from about 22 mg to about 30 mg of valdecoxib. In embodiments, the unit dosage form includes from about 24 mg to about 30 mg of valdecoxib. In embodiments, the unit dosage form includes from about 26 mg to about 30 mg of valdecoxib. In embodiments, the unit dosage form includes from about 28 mg to about 30 mg of valdecoxib.

In embodiments, the unit dosage form includes from about 2 mg to about 28 mg of valdecoxib. In embodiments, the unit dosage form includes from about 2 mg to about 26 mg of valdecoxib. In embodiments, the unit dosage form includes from about 2 mg to about 24 mg of valdecoxib. In embodiments, the unit dosage form includes from about 2 mg to about 22 mg of valdecoxib. In embodiments, the unit dosage form includes from about 2 mg to about 20 mg of valdecoxib. In embodiments, the unit dosage form includes from about 2 mg to about 18 mg of valdecoxib. In embodiments, the unit dosage form includes from about 2 mg to about 16 mg of valdecoxib. In embodiments, the unit dosage form includes from about 2 mg to about 14 mg of valdecoxib. In embodiments, the unit dosage form includes from about 2 mg to about 12 mg of valdecoxib. In embodiments, the unit dosage form includes from about 2 mg to about 10 mg of valdecoxib. In embodiments, the unit dosage form includes from about 2 mg to about 8 mg of valdecoxib. In embodiments, the unit dosage form includes from about 2 mg to about 6 mg of valdecoxib. In embodiments, the unit dosage form includes from about 2 mg to about 4 mg of valdecoxib. In embodiments, the unit dosage form includes from about 2 mg, 4 mg, 6 mg, 8 mg, 10 mg, 12 mg, 14 mg, 16 mg, 18 mg, 20 mg, 22 mg, 24 mg, 26 mg, 28 mg, or 30 mg of valdecoxib. In embodiments, the unit dosage form includes about 10 mg of valdecoxib. In embodiments, the unit dosage form includes 10 mg of valdecoxib. In embodiments, the unit dosage form includes about 20 mg of valdecoxib. In embodiments, the unit dosage form includes 20 mg of valdecoxib.

In embodiments, the unit dosage form includes from about 2 mg to about 70 mg of ketorolac. In embodiments, the unit dosage form includes from about 5 mg to about 70 mg of ketorolac. In embodiments, the unit dosage form includes from about 10 mg to about 70 mg of ketorolac. In embodiments, the unit dosage form includes from about 15 mg to about 70 mg of ketorolac. In embodiments, the unit dosage form includes from about 20 mg to about 70 mg of ketorolac. In embodiments, the unit dosage form includes from about 25 mg to about 70 mg of ketorolac. In embodiments, the unit dosage form includes from about 30 mg to about 70 mg of ketorolac. In embodiments, the unit dosage form includes from about 35 mg to about 70 mg of ketorolac. In embodiments, the unit dosage form includes from about 40 mg to about 70 mg of ketorolac. In embodiments, the unit dosage form includes from about 45 mg to about 70 mg of ketorolac. In embodiments, the unit dosage form includes from about 50 mg to about 70 mg of ketorolac. In embodiments, the unit dosage form includes from about 55 mg to about 70 mg of ketorolac. In embodiments, the unit dosage form includes from about 60 mg to about 70 mg of ketorolac. In embodiments, the unit dosage form includes from about 65 mg to about 70 mg of ketorolac.

In embodiments, the unit dosage form includes from about 2 mg to about 65 mg of ketorolac. In embodiments, the unit dosage form includes from about 2 mg to about 60 mg of ketorolac. In embodiments, the unit dosage form includes from about 2 mg to about 55 mg of ketorolac. In embodiments, the unit dosage form includes from about 2 mg to about 50 mg of ketorolac. In embodiments, the unit dosage form includes from about 2 mg to about 45 mg of ketorolac. In embodiments, the unit dosage form includes from about 2 mg to about 40 mg of ketorolac. In embodiments, the unit dosage form includes from about 2 mg to about 35 mg of ketorolac. In embodiments, the unit dosage form includes from about 2 mg to about 30 mg of ketorolac. In embodiments, the unit dosage form includes from about 2 mg to about 25 mg of ketorolac. In embodiments, the unit dosage form includes from about 2 mg to about 20 mg of ketorolac. In embodiments, the unit dosage form includes from about 2 mg to about 15 mg of ketorolac. In embodiments, the unit dosage form includes from about 2 mg to about 10 mg of ketorolac. In embodiments, the unit dosage form includes from about 2 mg to about 5 mg of ketorolac. In embodiments, the unit dosage form includes about 2 mg, 5 mg, 10 mg, 15 mg, 20 mg, 25 mg, 30 mg, 35 mg, 40 mg, 45 mg, 50 mg, 55 mg, 60 mg, or 65 mg of ketorolac. In embodiments, the unit dosage form includes about 30 mg of ketorolac. In embodiments, the unit dosage form includes 30 mg of ketorolac.

In embodiments, the unit dosage form includes from about 1 mg to about 70 mg of rofecoxib. In embodiments, the unit dosage form includes from about 5 mg to about 70 mg of rofecoxib. In embodiments, the unit dosage form includes from about 10 mg to about 70 mg of rofecoxib. In embodiments, the unit dosage form includes from about 15 mg to about 70 mg of rofecoxib. In embodiments, the unit dosage form includes from about 20 mg to about 70 mg of rofecoxib. In embodiments, the unit dosage form includes from about 25 mg to about 70 mg of rofecoxib. In embodiments, the unit dosage form includes from about 30 mg to about 70 mg of rofecoxib. In embodiments, the unit dosage form includes from about 35 mg to about 70 mg of rofecoxib. In embodiments, the unit dosage form includes from about 40 mg to about 70 mg of rofecoxib. In embodiments, the unit dosage form includes from about 45 mg to about 70 mg of rofecoxib. In embodiments, the unit dosage form includes from about 50 mg to about 70 mg of rofecoxib. In embodiments, the unit dosage form includes from about 55 mg to about 70 mg of rofecoxib. In embodiments, the unit dosage form includes from about 60 mg to about 70 mg of rofecoxib. In embodiments, the unit dosage form includes from about 65 mg to about 70 mg of rofecoxib.

In embodiments, the unit dosage form includes from about 1 mg to about 65 mg of rofecoxib. In embodiments, the unit dosage form includes from about 1 mg to about 60 mg of rofecoxib. In embodiments, the unit dosage form includes from about 1 mg to about 55 mg of rofecoxib. In embodiments, the unit dosage form includes from about 1 mg to about 50 mg of rofecoxib. In embodiments, the unit dosage form includes from about 1 mg to about 45 mg of rofecoxib. In embodiments, the unit dosage form includes from about 1 mg to about 40 mg of rofecoxib. In embodiments, the unit dosage form includes from about 1 mg to about 35 mg of rofecoxib. In embodiments, the unit dosage form includes from about 1 mg to about 30 mg of rofecoxib. In embodiments, the unit dosage form includes from about 1 mg to about 25 mg of rofecoxib. In embodiments, the unit dosage form includes from about 1 mg to about 20 mg of rofecoxib. In embodiments, the unit dosage form includes from about 1 mg to about 15 mg of rofecoxib. In embodiments, the unit dosage form includes from about 1 mg to about 10 mg of rofecoxib. In embodiments, the unit dosage form includes from about 1 mg to about 5 mg of rofecoxib. In embodiments, the unit dosage form includes about 1 mg, 5 mg, 10 mg, 15 mg, 20 mg, 25 mg, 30 mg, 35 mg, 40 mg, 45 mg, 50 mg, 55 mg, 60 mg, 65 mg, or 70 mg of rofecoxib. In embodiments, the unit dosage form includes about 12.5 mg of rofecoxib. In embodiments, the unit dosage form includes 12.5 mg of rofecoxib. In embodiments, the unit dosage form includes about 25 mg of rofecoxib. In embodiments, the unit dosage form includes 25 mg of rofecoxib.

In embodiments, the pharmaceutical kit provided herein further includes an epidermal growth factor receptor (EGFR) inhibitor. In embodiments, the EGFR inhibitor is in a discrete composition of a EGFR inhibitor and is separate from other dosage forms (e.g., first dosage form and the second dosage form). In embodiments, the dosage form including the EGFR inhibitor does not include any other active agents. In embodiments, the dosage form including the EGFR inhibitor does not include any other therapeutic agent. In embodiments, the dosage form including the EGFR inhibitor does not include any other anti-cancer agent. In embodiments, the EGFR inhibitor is in a unit dosage form including a plurality of therapeutic agents of the combination therapy. In embodiments, the unit dosage form includes an EGFR inhibitor and a BRAF inhibitor. In embodiments, the unit dosage form including the EGFR inhibitor and the BRAF inhibitor does not include any other active agents. In embodiments, the unit dosage form including the EGFR inhibitor and the BRAF inhibitor does not include any other therapeutic agent. In embodiments, the unit dosage form including the EGFR inhibitor and the BRAF inhibitor does not include any other anti-cancer agent. In embodiments, the unit dosage form includes an EGFR inhibitor, a BRAF inhibitor, and a COX-2 inhibitor. In embodiments, the unit dosage form including the EGFR inhibitor, the BRAF inhibitor, and the COX-2 inhibitor does not include any other active agents. In embodiments, the unit dosage form including the EGFR inhibitor, the BRAF inhibitor, and the COX-2 inhibitor does not include any other therapeutic agent. In embodiments, the unit dosage form including the EGFR inhibitor, the BRAF inhibitor, and the COX-2 inhibitor does not include any other anti-cancer agent.

In embodiments, the EGFR inhibitor is panitumumab, cetuximab, gefitinib, erlotinib, neratinib, lapatinib, necitumumab, osimertinib, dacomitinib, mobocertinib, vandetanib, or a combination thereof. In embodiments, the EGFR inhibitor is panitumumab, cetuximab, gefitinib, erlotinib, neratinib, lapatinib, necitumumab, or a combination thereof. In embodiments, the EGFR inhibitor is panitumumab. In embodiments, the EGFR inhibitor is cetuximab. In embodiments, the EGFR inhibitor is gefitinib.

In embodiments, the unit dosage form includes from about 10 mg to about 600 mg of panitumumab. In embodiments, the unit dosage form includes from about 50 mg to about 600 mg of panitumumab. In embodiments, the unit dosage form includes from about 100 mg to about 600 mg of panitumumab. In embodiments, the unit dosage form includes from about 150 mg to about 600 mg of panitumumab. In embodiments, the unit dosage form includes from about 200 mg to about 600 mg of panitumumab. In embodiments, the unit dosage form includes from about 250 mg to about 600 mg of panitumumab. In embodiments, the unit dosage form includes from about 300 mg to about 600 mg of panitumumab. In embodiments, the unit dosage form includes from about 350 mg to about 600 mg of panitumumab. In embodiments, the unit dosage form includes from about 400 mg to about 600 mg of panitumumab. In embodiments, the unit dosage form includes from about 450 mg to about 600 mg of panitumumab. In embodiments, the unit dosage form includes from about 500 mg to about 600 mg of panitumumab. In embodiments, the unit dosage form includes from about 550 mg to about 600 mg of panitumumab.

In embodiments, the unit dosage form includes from about 10 mg to about 550 mg of panitumumab. In embodiments, the unit dosage form includes from about 10 mg to about 500 mg of panitumumab. In embodiments, the unit dosage form includes from about 10 mg to about 450 mg of panitumumab. In embodiments, the unit dosage form includes from about 10 mg to about 400 mg of panitumumab. In embodiments, the unit dosage form includes from about 10 mg to about 350 mg of panitumumab. In embodiments, the unit dosage form includes from about 10 mg to about 300 mg of panitumumab. In embodiments, the unit dosage form includes from about 10 mg to about 250 mg of panitumumab. In embodiments, the unit dosage form includes from about 10 mg to about 200 mg of panitumumab. In embodiments, the unit dosage form includes from about 10 mg to about 150 mg of panitumumab. In embodiments, the unit dosage form includes from about 10 mg to about 100 mg of panitumumab. In embodiments, the unit dosage form includes from about 10 mg to about 50 mg of panitumumab. In embodiments, the unit dosage form includes about 10 mg, 50 mg, 100 mg, 150 mg, 200 mg, 250 mg, 300 mg, 350 mg, 400 mg, 450 mg, 500 mg, 550 mg, or 600 mg of panitumumab.

In embodiments, the unit dosage form includes from about 10 mg to about 310 mg of cetuximab. In embodiments, the unit dosage form includes from about 30 mg to about 310 mg of cetuximab. In embodiments, the unit dosage form includes from about 50 mg to about 310 mg of cetuximab. In embodiments, the unit dosage form includes from about 70 mg to about 310 mg of cetuximab. In embodiments, the unit dosage form includes from about 90 mg to about 310 mg of cetuximab. In embodiments, the unit dosage form includes from about 110 mg to about 310 mg of cetuximab. In embodiments, the unit dosage form includes from about 130 mg to about 310 mg of cetuximab. In embodiments, the unit dosage form includes from about 150 mg to about 310 mg of cetuximab. In embodiments, the unit dosage form includes from about 170 mg to about 310 mg of cetuximab. In embodiments, the unit dosage form includes from about 190 mg to about 310 mg of cetuximab. In embodiments, the unit dosage form includes from about 210 mg to about 310 mg of cetuximab. In embodiments, the unit dosage form includes from about 230 mg to about 310 mg of cetuximab. In embodiments, the unit dosage form includes from about 250 mg to about 310 mg of cetuximab. In embodiments, the unit dosage form includes from about 270 mg to about 310 mg of cetuximab. In embodiments, the unit dosage form includes from about 290 mg to about 310 mg of cetuximab.

In embodiments, the unit dosage form includes from about 10 mg to about 290 mg of cetuximab. In embodiments, the unit dosage form includes from about 10 mg to about 270 mg of cetuximab. In embodiments, the unit dosage form includes from about 10 mg to about 250 mg of cetuximab. In embodiments, the unit dosage form includes from about 10 mg to about 230 mg of cetuximab. In embodiments, the unit dosage form includes from about 10 mg to about 210 mg of cetuximab. In embodiments, the unit dosage form includes from about 10 mg to about 190 mg of cetuximab. In embodiments, the unit dosage form includes from about 10 mg to about 170 mg of cetuximab. In embodiments, the unit dosage form includes from about 10 mg to about 150 mg of cetuximab. In embodiments, the unit dosage form includes from about 10 mg to about 130 mg of cetuximab. In embodiments, the unit dosage form includes from about 10 mg to about 110 mg of cetuximab. In embodiments, the unit dosage form includes from about 10 mg to about 90 mg of cetuximab. In embodiments, the unit dosage form includes from about 10 mg to about 70 mg of cetuximab. In embodiments, the unit dosage form includes from about 10 mg to about 50 mg of cetuximab. In embodiments, the unit dosage form includes from about 10 mg to about 30 mg of cetuximab. In embodiments, the unit dosage form includes about 10 mg, 30 mg, 50 mg, 70 mg, 90 mg, 110 mg, 130 mg, 150 mg, 170 mg, 190 mg, 210 mg, 230 mg, 250 mg, 270 mg, 290 or 310 mg of cetuximab. In embodiments, the unit dosage form includes about 100 mg of cetuximab. In embodiments, the unit dosage form includes 100 mg of cetuximab. In embodiments, the unit dosage form includes about 200 mg of cetuximab. In embodiments, the unit dosage form includes 200 mg of cetuximab.

In embodiments, the unit dosage form includes from about 10 mg to about 310 mg of gefitinib. In embodiments, the unit dosage form includes from about 30 mg to about 310 mg of gefitinib. In embodiments, the unit dosage form includes from about 50 mg to about 310 mg of gefitinib. In embodiments, the unit dosage form includes from about 70 mg to about 310 mg of gefitinib. In embodiments, the unit dosage form includes from about 90 mg to about 310 mg of gefitinib. In embodiments, the unit dosage form includes from about 110 mg to about 310 mg of gefitinib. In embodiments, the unit dosage form includes from about 130 mg to about 310 mg of gefitinib. In embodiments, the unit dosage form includes from about 150 mg to about 310 mg of gefitinib. In embodiments, the unit dosage form includes from about 170 mg to about 310 mg of gefitinib. In embodiments, the unit dosage form includes from about 190 mg to about 310 mg of gefitinib. In embodiments, the unit dosage form includes from about 210 mg to about 310 mg of gefitinib. In embodiments, the unit dosage form includes from about 230 mg to about 310 mg of gefitinib. In embodiments, the unit dosage form includes from about 250 mg to about 310 mg of gefitinib. In embodiments, the unit dosage form includes from about 270 mg to about 310 mg of gefitinib. In embodiments, the unit dosage form includes from about 290 mg to about 310 mg of gefitinib.

In embodiments, the unit dosage form includes from about 10 mg to about 290 mg of gefitinib. In embodiments, the unit dosage form includes from about 10 mg to about 270 mg of gefitinib. In embodiments, the unit dosage form includes from about 10 mg to about 250 mg of gefitinib. In embodiments, the unit dosage form includes from about 10 mg to about 230 mg of gefitinib. In embodiments, the unit dosage form includes from about 10 mg to about 210 mg of gefitinib. In embodiments, the unit dosage form includes from about 10 mg to about 190 mg of gefitinib. In embodiments, the unit dosage form includes from about 10 mg to about 170 mg of gefitinib. In embodiments, the unit dosage form includes from about 10 mg to about 150 mg of gefitinib. In embodiments, the unit dosage form includes from about 10 mg to about 130 mg of gefitinib. In embodiments, the unit dosage form includes from about 10 mg to about 110 mg of gefitinib. In embodiments, the unit dosage form includes from about 10 mg to about 90 mg of gefitinib. In embodiments, the unit dosage form includes from about 10 mg to about 70 mg of gefitinib. In embodiments, the unit dosage form includes from about 10 mg to about 50 mg of gefitinib. In embodiments, the unit dosage form includes from about 10 mg to about 30 mg of gefitinib. In embodiments, the unit dosage form includes about 10 mg, 30 mg, 50 mg, 70 mg, 90 mg, 110 mg, 130 mg, 150 mg, 170 mg, 190 mg, 210 mg, 230 mg, 250 mg, 270 mg, 290 or 310 mg of gefitinib. In embodiments, the unit dosage form includes about 250 mg of gefitinib. In embodiments, the unit dosage form includes 250 mg of gefitinib.

In embodiments, the unit dosage form includes from about 5 mg to about 205 mg of erlotinib. In embodiments, the unit dosage form includes from about 25 mg to about 205 mg of erlotinib. In embodiments, the unit dosage form includes from about 45 mg to about 205 mg of erlotinib. In embodiments, the unit dosage form includes from about 65 mg to about 205 mg of erlotinib. In embodiments, the unit dosage form includes from about 85 mg to about 205 mg of erlotinib. In embodiments, the unit dosage form includes from about 105 mg to about 205 mg of erlotinib. In embodiments, the unit dosage form includes from about 125 mg to about 205 mg of erlotinib. In embodiments, the unit dosage form includes from about 145 mg to about 205 mg of erlotinib. In embodiments, the unit dosage form includes from about 165 mg to about 205 mg of erlotinib. In embodiments, the unit dosage form includes from about 185 mg to about 205 mg of erlotinib.

In embodiments, the unit dosage form includes from about 5 mg to about 205 mg of erlotinib. In embodiments, the unit dosage form includes from about 5 mg to about 185 mg of erlotinib. In embodiments, the unit dosage form includes from about 5 mg to about 165 mg of erlotinib. In embodiments, the unit dosage form includes from about 5 mg to about 145 mg of erlotinib. In embodiments, the unit dosage form includes from about 5 mg to about 125 mg of erlotinib. In embodiments, the unit dosage form includes from about 5 mg to about 105 mg of erlotinib. In embodiments, the unit dosage form includes from about 5 mg to about 85 mg of erlotinib. In embodiments, the unit dosage form includes from about 5 mg to about 65 mg of erlotinib. In embodiments, the unit dosage form includes from about 5 mg to about 45 mg of erlotinib. In embodiments, the unit dosage form includes from about 5 mg to about 25 mg of erlotinib. In embodiments, the unit dosage form includes about 5 mg, 25 mg, 45 mg, 65 mg, 85 mg, 105 mg, 125 mg, 145 mg, 165 mg, 185 mg, or 205 mg of erlotinib. In embodiments, the unit dosage form includes about 25 mg of erlotinib. In embodiments, the unit dosage form includes 25 mg of erlotinib. In embodiments, the unit dosage form includes about 100 mg of erlotinib. In embodiments, the unit dosage form includes 100 mg of erlotinib. In embodiments, the unit dosage form includes about 150 mg of erlotinib. In embodiments, the unit dosage form includes 150 mg of erlotinib.

In embodiments, the pharmaceutical composition provided herein further includes a SRC inhibitor. In embodiments, the SRC inhibitor is in a discrete composition of a SRC inhibitor and is separate from other dosage forms (e.g., first dosage form and the second dosage form). In embodiments, the dosage form including the SRC inhibitor does not include any other active agents. In embodiments, the dosage form including the SRC inhibitor does not include any other therapeutic agent. In embodiments, the dosage form including the SRC inhibitor does not include any other anti-cancer agent. In embodiments, the SRC inhibitor is in a unit dosage form including a plurality of therapeutic agents of the combination therapy. In embodiments, the unit dosage form includes a SRC inhibitor and a BRAF inhibitor. In embodiments, the unit dosage form including the SRC inhibitor and the BRAF inhibitor does not include any other active agents. In embodiments, the unit dosage form including the SRC inhibitor and the BRAF inhibitor does not include any other therapeutic agent. In embodiments, the unit dosage form including the SRC inhibitor and the BRAF inhibitor does not include any other anti-cancer agent. In embodiments, the unit dosage form includes a SRC inhibitor, a BRAF inhibitor, and a COX-2 inhibitor. In embodiments, the unit dosage form including the SRC inhibitor, the BRAF inhibitor, and the COX-2 inhibitor does not include any other active agents. In embodiments, the unit dosage form including the SRC inhibitor, the BRAF inhibitor, and the COX-2 inhibitor does not include any other therapeutic agent. In embodiments, the unit dosage form including the SRC inhibitor, the BRAF inhibitor, and the COX-2 inhibitor does not include any other anti-cancer agent.

In embodiments, the SRC inhibitor is dasatinib, saracatinib, ponatinib, bosutinib, pelitinib, resveratrol, KX2-391, NVP-BHG712, ENMD-2076, PP2, PP121, PP1, 2,3-Methylenedioxy-beta-nitrostyrene (MNS), 1-NM-PP1, NPK1-IN-2, XL228, DGY-06-116, Elzovanitinib, eCF506, RK 24466, 1 Naphtyl PP1 (1-NA-PP1), AMG-47a, Src Inhibitor 1, KX1-004, Myristic Acid, 7-Hydroxy-4-chromone, dasatinib hydrochloride, UM-164, repotrectinib, CCT196969, ON123300, SU6656, dasatinib monohydrate, WH-4-023, doramapimod, TPX-0046, dehydroabietic acid, ginkogolic acid C17:1, AD80, quercetin, or a combination thereof. In embodiments, the SRC inhibitor is dasatinib, saracatinib, bosutinib or a combination thereof.

In embodiments, the unit dosage form includes from about 2 mg to about 200 mg of dasatinib. In embodiments, the unit dosage form includes from about 10 mg to about 200 mg of dasatinib. In embodiments, the unit dosage form includes from about 20 mg to about 200 mg of dasatinib. In embodiments, the unit dosage form includes from about 30 mg to about 200 mg of dasatinib. In embodiments, the unit dosage form includes from about 40 mg to about 200 mg of dasatinib. In embodiments, the unit dosage form includes from about 50 mg to about 200 mg of dasatinib. In embodiments, the unit dosage form includes from about 60 mg to about 200 mg of dasatinib. In embodiments, the unit dosage form includes from about 70 mg to about 200 mg of dasatinib. In embodiments, the unit dosage form includes from about 80 mg to about 200 mg of dasatinib. In embodiments, the unit dosage form includes from about 90 mg to about 200 mg of dasatinib. In embodiments, the unit dosage form includes from about 100 mg to about 200 mg of dasatinib. In embodiments, the unit dosage form includes from about 110 mg to about 200 mg of dasatinib. In embodiments, the unit dosage form includes from about 120 mg to about 200 mg of dasatinib. In embodiments, the unit dosage form includes from about 130 mg to about 200 mg of dasatinib. In embodiments, the unit dosage form includes from about 140 mg to about 200 mg of dasatinib. In embodiments, the unit dosage form includes from about 150 mg to about 200 mg of dasatinib. In embodiments, the unit dosage form includes from about 160 mg to about 200 mg of dasatinib. In embodiments, the unit dosage form includes from about 170 mg to about 200 mg of dasatinib. In embodiments, the unit dosage form includes from about 180 mg to about 200 mg of dasatinib. In embodiments, the unit dosage form includes from about 190 mg to about 200 mg of dasatinib.

In embodiments, the unit dosage form includes from about 2 mg to about 190 mg of dasatinib. In embodiments, the unit dosage form includes from about 2 mg to about 180 mg of dasatinib. In embodiments, the unit dosage form includes from about 2 mg to about 170 mg of dasatinib. In embodiments, the unit dosage form includes from about 2 mg to about 160 mg of dasatinib. In embodiments, the unit dosage form includes from about 2 mg to about 150 mg of dasatinib. In embodiments, the unit dosage form includes from about 2 mg to about 140 mg of dasatinib. In embodiments, the unit dosage form includes from about 2 mg to about 130 mg of dasatinib. In embodiments, the unit dosage form includes from about 2 mg to about 120 mg of dasatinib. In embodiments, the unit dosage form includes from about 2 mg to about 110 mg of dasatinib. In embodiments, the unit dosage form includes from about 2 mg to about 100 mg of dasatinib. In embodiments, the unit dosage form includes from about 2 mg to about 90 mg of dasatinib. In embodiments, the unit dosage form includes from about 2 mg to about 80 mg of dasatinib. In embodiments, the unit dosage form includes from about 2 mg to about 70 mg of dasatinib. In embodiments, the unit dosage form includes from about 2 mg to about 60 mg of dasatinib. In embodiments, the unit dosage form includes from about 2 mg to about 50 mg of dasatinib. In embodiments, the unit dosage form includes from about 2 mg to about 40 mg of dasatinib. In embodiments, the unit dosage form includes from about 2 mg to about 30 mg of dasatinib. In embodiments, the unit dosage form includes from about 2 mg to about 20 mg of dasatinib. In embodiments, the unit dosage form includes from about 2 mg to about 10 mg of dasatinib. In embodiments, the unit dosage form includes about 2 mg, 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, or 200 mg of dasatinib. In embodiments, the unit dosage form includes about 20 mg of dasatinib. In embodiments, the unit dosage form includes 20 mg of dasatinib. In embodiments, the unit dosage form includes about 50 mg of dasatinib. In embodiments, the unit dosage form includes 50 mg of dasatinib. In embodiments, the unit dosage form includes about 70 mg of dasatinib. In embodiments, the unit dosage form includes 70 mg of dasatinib. In embodiments, the unit dosage form includes about 80 mg of dasatinib. In embodiments, the unit dosage form includes 80 mg of dasatinib. In embodiments, the unit dosage form includes about 100 mg of dasatinib. In embodiments, the unit dosage form includes 100 mg of dasatinib. In embodiments, the unit dosage form includes about 140 mg of dasatinib. In embodiments, the unit dosage form includes 140 mg of dasatinib.

In embodiments, the unit dosage form includes from about 10 mg to about 310 mg of saracatinib. In embodiments, the unit dosage form includes from about 30 mg to about 310 mg of saracatinib. In embodiments, the unit dosage form includes from about 50 mg to about 310 mg of saracatinib. In embodiments, the unit dosage form includes from about 70 mg to about 310 mg of saracatinib. In embodiments, the unit dosage form includes from about 90 mg to about 310 mg of saracatinib. In embodiments, the unit dosage form includes from about 110 mg to about 310 mg of saracatinib. In embodiments, the unit dosage form includes from about 130 mg to about 310 mg of saracatinib. In embodiments, the unit dosage form includes from about 150 mg to about 310 mg of saracatinib. In embodiments, the unit dosage form includes from about 170 mg to about 310 mg of saracatinib. In embodiments, the unit dosage form includes from about 190 mg to about 310 mg of saracatinib. In embodiments, the unit dosage form includes from about 210 mg to about 310 mg of saracatinib. In embodiments, the unit dosage form includes from about 230 mg to about 310 mg of saracatinib. In embodiments, the unit dosage form includes from about 250 mg to about 310 mg of saracatinib. In embodiments, the unit dosage form includes from about 270 mg to about 310 mg of saracatinib. In embodiments, the unit dosage form includes from about 290 mg to about 310 mg of saracatinib.

In embodiments, the unit dosage form includes from about 10 mg to about 290 mg of saracatinib. In embodiments, the unit dosage form includes from about 10 mg to about 270 mg of saracatinib. In embodiments, the unit dosage form includes from about 10 mg to about 250 mg of saracatinib. In embodiments, the unit dosage form includes from about 10 mg to about 230 mg of saracatinib. In embodiments, the unit dosage form includes from about 10 mg to about 210 mg of saracatinib. In embodiments, the unit dosage form includes from about 10 mg to about 190 mg of saracatinib. In embodiments, the unit dosage form includes from about 10 mg to about 170 mg of saracatinib. In embodiments, the unit dosage form includes from about 10 mg to about 150 mg of saracatinib. In embodiments, the unit dosage form includes from about 10 mg to about 130 mg of saracatinib. In embodiments, the unit dosage form includes from about 10 mg to about 110 mg of saracatinib. In embodiments, the unit dosage form includes from about 10 mg to about 90 mg of saracatinib. In embodiments, the unit dosage form includes from about 10 mg to about 70 mg of saracatinib. In embodiments, the unit dosage form includes from about 10 mg to about 50 mg of saracatinib. In embodiments, the unit dosage form includes from about 10 mg to about 30 mg of saracatinib. In embodiments, the unit dosage form includes about 10 mg, 30 mg, 50 mg, 70 mg, 90 mg, 110 mg, 130 mg, 150 mg, 170 mg, 190 mg, 210 mg, 230 mg, 250 mg, 270 mg, 290 or 310 mg of saracatinib.

In embodiments, the unit dosage form includes from about 25 mg to about 600 mg of bosutinib. In embodiments, the unit dosage form includes from about 50 mg to about 600 mg of bosutinib. In embodiments, the unit dosage form includes from about 75 mg to about 600 mg of bosutinib. In embodiments, the unit dosage form includes from about 100 mg to about 600 mg of bosutinib. In embodiments, the unit dosage form includes from about 125 mg to about 600 mg of bosutinib. In embodiments, the unit dosage form includes from about 150 mg to about 600 mg of bosutinib. In embodiments, the unit dosage form includes from about 175 mg to about 600 mg of bosutinib. In embodiments, the unit dosage form includes from about 200 mg to about 600 mg of bosutinib. In embodiments, the unit dosage form includes from about 225 mg to about 600 mg of bosutinib. In embodiments, the unit dosage form includes from about 250 mg to about 600 mg of bosutinib. In embodiments, the unit dosage form includes from about 275 mg to about 600 mg of bosutinib. In embodiments, the unit dosage form includes from about 300 mg to about 600 mg of bosutinib. In embodiments, the unit dosage form includes from about 325 mg to about 600 mg of bosutinib. In embodiments, the unit dosage form includes from about 350 mg to about 600 mg of bosutinib. In embodiments, the unit dosage form includes from about 375 mg to about 600 mg of bosutinib. In embodiments, the unit dosage form includes from about 400 mg to about 600 mg of bosutinib. In embodiments, the unit dosage form includes from about 425 mg to about 600 mg of bosutinib. In embodiments, the unit dosage form includes from about 450 mg to about 600 mg of bosutinib. In embodiments, the unit dosage form includes from about 475 mg to about 600 mg of bosutinib. In embodiments, the unit dosage form includes from about 500 mg to about 600 mg of bosutinib. In embodiments, the unit dosage form includes from about 525 mg to about 600 mg of bosutinib. In embodiments, the unit dosage form includes from about 550 mg to about 600 mg of bosutinib. In embodiments, the unit dosage form includes from about 575 mg to about 600 mg of bosutinib.

In embodiments, the unit dosage form includes from about 25 mg to about 575 mg of bosutinib. In embodiments, the unit dosage form includes from about 25 mg to about 550 mg of bosutinib. In embodiments, the unit dosage form includes from about 25 mg to about 525 mg of bosutinib. In embodiments, the unit dosage form includes from about 25 mg to about 500 mg of bosutinib. In embodiments, the unit dosage form includes from about 25 mg to about 475 mg of bosutinib. In embodiments, the unit dosage form includes from about 25 mg to about 450 mg of bosutinib. In embodiments, the unit dosage form includes from about 25 mg to about 425 mg of bosutinib. In embodiments, the unit dosage form includes from about 25 mg to about 400 mg of bosutinib. In embodiments, the unit dosage form includes from about 25 mg to about 375 mg of bosutinib. In embodiments, the unit dosage form includes from about 25 mg to about 350 mg of bosutinib. In embodiments, the unit dosage form includes from about 25 mg to about 325 mg of bosutinib. In embodiments, the unit dosage form includes from about 25 mg to about 300 mg of bosutinib. In embodiments, the unit dosage form includes from about 25 mg to about 275 mg of bosutinib. In embodiments, the unit dosage form includes from about 25 mg to about 250 mg of bosutinib. In embodiments, the unit dosage form includes from about 25 mg to about 225 mg of bosutinib. In embodiments, the unit dosage form includes from about 25 mg to about 200 mg of bosutinib. In embodiments, the unit dosage form includes from about 25 mg to about 175 mg of bosutinib. In embodiments, the unit dosage form includes from about 25 mg to about 150 mg of bosutinib. In embodiments, the unit dosage form includes from about 25 mg to about 125 mg of bosutinib. In embodiments, the unit dosage form includes from about 25 mg to about 100 mg of bosutinib. In embodiments, the unit dosage form includes from about 25 mg to about 75 mg of bosutinib. In embodiments, the unit dosage form includes from about 25 mg to about 50 mg of bosutinib. In embodiments, the unit dosage form includes about 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, or 600 mg of bosutinib. In embodiments, the unit dosage form includes about 100 mg of bosutinib. In embodiments, the unit dosage form includes 100 mg of bosutinib. In embodiments, the unit dosage form includes about 500 mg of bosutinib. In embodiments, the unit dosage form includes 500 mg of bosutinib.

In embodiments, the unit dosage form includes from about 5 mg to about 50 mg of ponatinib. In embodiments, the unit dosage form includes from about 10 mg to about 50 mg of ponatinib. In embodiments, the unit dosage form includes from about 15 mg to about 50 mg of ponatinib. In embodiments, the unit dosage form includes from about 20 mg to about 50 mg of ponatinib. In embodiments, the unit dosage form includes from about 25 mg to about 50 mg of ponatinib. In embodiments, the unit dosage form includes from about 30 mg to about 50 mg of ponatinib. In embodiments, the unit dosage form includes from about 35 mg to about 50 mg of ponatinib. In embodiments, the unit dosage form includes from about 40 mg to about 50 mg of ponatinib. In embodiments, the unit dosage form includes from about 45 mg to about 50 mg of ponatinib.

In embodiments, the unit dosage form includes from about 5 mg to about 45 mg of ponatinib. In embodiments, the unit dosage form includes from about 5 mg to about 40 mg of ponatinib. In embodiments, the unit dosage form includes from about 5 mg to about 35 mg of ponatinib. In embodiments, the unit dosage form includes from about 5 mg to about 30 mg of ponatinib. In embodiments, the unit dosage form includes from about 5 mg to about 25 mg of ponatinib. In embodiments, the unit dosage form includes from about 5 mg to about 20 mg of ponatinib. In embodiments, the unit dosage form includes from about 5 mg to about 15 mg of ponatinib. In embodiments, the unit dosage form includes from about 5 mg to about 10 mg of ponatinib. In embodiments, the unit dosage form includes about 5 mg, 10 mg, 15 mg, 20 mg, 25 mg, 30 mg, 35 mg, 40 mg, 45 mg, or 50 mg of ponatinib. In embodiments, the unit dosage form includes about 15 mg of ponatinib. In embodiments, the unit dosage form includes 15 mg of ponatinib. In embodiments, the unit dosage form includes about 45 mg of ponatinib. In embodiments, the unit dosage form includes 45 mg of ponatinib.

In embodiments, the unit dosage form includes from about 25 mg to about 450 mg of imatinib. In embodiments, the unit dosage form includes from about 50 mg to about 450 mg of imatinib. In embodiments, the unit dosage form includes from about 75 mg to about 450 mg of imatinib. In embodiments, the unit dosage form includes from about 100 mg to about 450 mg of imatinib. In embodiments, the unit dosage form includes from about 125 mg to about 450 mg of imatinib. In embodiments, the unit dosage form includes from about 150 mg to about 450 mg of imatinib. In embodiments, the unit dosage form includes from about 125 mg to about 450 mg of imatinib. In embodiments, the unit dosage form includes from about 150 mg to about 450 mg of imatinib. In embodiments, the unit dosage form includes from about 175 mg to about 450 mg of imatinib. In embodiments, the unit dosage form includes from about 200 mg to about 450 mg of imatinib. In embodiments, the unit dosage form includes from about 225 mg to about 450 mg of imatinib. In embodiments, the unit dosage form includes from about 250 mg to about 450 mg of imatinib. In embodiments, the unit dosage form includes from about 275 mg to about 450 mg of imatinib. In embodiments, the unit dosage form includes from about 300 mg to about 450 mg of imatinib. In embodiments, the unit dosage form includes from about 325 mg to about 450 mg of imatinib. In embodiments, the unit dosage form includes from about 350 mg to about 450 mg of imatinib. In embodiments, the unit dosage form includes from about 375 mg to about 450 mg of imatinib. In embodiments, the unit dosage form includes from about 400 mg to about 450 mg of imatinib. In embodiments, the unit dosage form includes from about 425 mg to about 450 mg of imatinib.

In embodiments, the unit dosage form includes from about 25 mg to about 425 mg of imatinib. In embodiments, the unit dosage form includes from about 25 mg to about 400 mg of imatinib. In embodiments, the unit dosage form includes from about 25 mg to about 375 mg of imatinib. In embodiments, the unit dosage form includes from about 25 mg to about 350 mg of imatinib. In embodiments, the unit dosage form includes from about 25 mg to about 325 mg of imatinib. In embodiments, the unit dosage form includes from about 25 mg to about 300 mg of imatinib. In embodiments, the unit dosage form includes from about 25 mg to about 275 mg of imatinib. In embodiments, the unit dosage form includes from about 25 mg to about 250 mg of imatinib. In embodiments, the unit dosage form includes from about 25 mg to about 225 mg of imatinib. In embodiments, the unit dosage form includes from about 25 mg to about 200 mg of imatinib. In embodiments, the unit dosage form includes from about 25 mg to about 175 mg of imatinib. In embodiments, the unit dosage form includes from about 25 mg to about 150 mg of imatinib. In embodiments, the unit dosage form includes from about 25 mg to about 125 mg of imatinib. In embodiments, the unit dosage form includes from about 25 mg to about 100 mg of imatinib. In embodiments, the unit dosage form includes from about 25 mg to about 75 mg of imatinib. In embodiments, the unit dosage form includes from about 25 mg to about 50 mg of imatinib. In embodiments, the unit dosage form includes about 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, or 450 mg of imatinib. In embodiments, the unit dosage form includes about 100 mg of imatinib. In embodiments, the unit dosage form includes 100 mg of imatinib. In embodiments, the unit dosage form includes about 400 mg of imatinib. In embodiments, the unit dosage form includes 400 mg of imatinib.

For the pharmaceutical kit provided herein, in embodiments, the unit dosage form includes less than than the therapeutically effective amount of the BRAF axis inhibitor provided herein when used separately from the COX-2 inhibitor. In embodiments, the unit dosage form includes less than than the therapeutically effective amount of the COX-2 inhibitor provided herein when used separately from the BRAF axis inhibitor. In embodiments, the unit dosage form includes less than the therapeutically effective amount of the COX-2 axis inhibitor and the BRAF axis inhibitor when used separately from each other.

In embodiments, the pharmaceutical kit further includes instructions for treatment of cancer. In embodiments, the pharmaceutical kit includes instructions for treatment of a BRAF resistant cancer. In embodiments, the pharmaceutical kit includes instructions for treatment of a BRAF intrinsically resistant cancer. In embodiments, the BRAF intrinsically resistant cancer is colorectal cancer, thyroid cancer, glioblastoma, sarcoma, or hepto-sarcoma. In embodiments, the BRAF intrinsically resistant cancer is colorectal cancer.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EMBODIMENTS

Embodiment 1. A method of treating a serine/threonine-protein kinase B-Raf (BRAF) intrinsically resistant cancer in a subject, comprising administering to the subject a combined effective amount of a BRAF axis inhibitor and a cyclooxygenase-2 (COX-2) inhibitor.
Embodiment 2. The method of embodiment 1, wherein the BRAF axis inhibitor comprises a BRAF inhibitor, a MEK inhibitor, an ERK inhibitor, or a combination thereof.
Embodiment 3. The method of embodiment 2, wherein the BRAF axis inhibitor comprises a BRAF inhibitor and an MEK inhibitor.
Embodiment 4. The method of embodiment 2, wherein the BRAF axis inhibitor comprises a BRAF inhibitor and an ERK inhibitor.
Embodiment 5. The method of any one of embodiments 2-4, wherein the BRAF axis inhibitor comprises a BRAF inhibitor, a MEK inhibitor, and an ERK inhibitor.
Embodiment 6. The method of any one of embodiments 2, 3, or 5, wherein the MEK inhibitor is trametinib, binimetinib, cobimetinib, selumetinib, or a combination thereof.
Embodiment 7. The method of embodiment 6, wherein the MEK inhibitor is trametinib.
Embodiment 8. The method of any one of embodiments 2, 4, or 5, wherein the ERK inhibitor is ulixertinib (BVD-523), ravoxertinib (GDC-0994), LY3214996, LTT462, or a combination thereof.
Embodiment 9. The method of any one of embodiments 2-5, wherein the BRAF inhibitor is encorafenib, vemurafenib, or dabrafenib, or a combination thereof.
Embodiment 10. The method of embodiment 9, wherein the BRAF inhibitor is vemurafenib or encorafenib.
Embodiment 11. The method of any one of embodiments 1-10, wherein the COX-2 inhibitor is celecoxib, valdecoxib, ketorolac, rofecoxib, or a combination thereof.
Embodiment 12. The method of embodiment 11, wherein the COX-2 inhibitor is celecoxib.
Embodiment 13. The method of any one of embodiments 1-12, wherein the BRAF axis inhibitor and the COX-2 inhibitor are administered simultaneously.
Embodiment 14. The method of any one of embodiments 1-12, wherein the BRAF axis inhibitor and the COX-2 inhibitor are administered sequentially.
Embodiment 15. The method of any one of embodiments 1-14, further comprising administering to the subject an epidermal growth factor receptor (EGFR) inhibitor.
Embodiment 16. The method of embodiment 15, wherein the EGFR inhibitor is panitumumab, cetuximab, gefitinib, erlotinib, neratinib, lapatinib, necitumumab, or a combination thereof.
Embodiment 17. The method of any one of embodiments 1-16, further comprising administering to the subject a SRC inhibitor.
Embodiment 18. The method of embodiment 17, wherein the SRC inhibitor is dasatinib, saracatinib, bosutinib, ponatinib, imatinib, or a combination thereof.
Embodiment 19. The method of any one of embodiments 1-18, wherein the BRAF intrinsically resistant cancer is colorectal cancer, thyroid cancer, glioblastoma, sarcoma, or hepto-sarcoma.
Embodiment 20. The method of any one of embodiments 1-19, wherein the BRAF intrinsically resistant cancer is colorectal cancer.
Embodiment 21. A pharmaceutical kit comprising: i) a serine/threonine-protein kinase B-Raf (BRAF) axis inhibitor; and ii) a and a cyclooxygenase-2 (COX-2) inhibitor
Embodiment 22. The pharmaceutical kit of embodiment 21, wherein the BRAF axis inhibitor is a BRAF inhibitor, a MEK inhibitor, an ERK inhibitor, or a combination thereof.
Embodiment 23. The pharmaceutical kit of embodiment 22, wherein the BRAF axis inhibitor comprises a BRAF inhibitor and a MEK inhibitor.
Embodiment 24. The pharmaceutical kit of embodiment 22, wherein the BRAF axis inhibitor comprises a BRAF inhibitor and an ERK inhibitor.
Embodiment 25. The pharmaceutical kit of any one of embodiments 22-24, wherein the BRAF axis inhibitor comprises a BRAF inhibitor, a MEK inhibitor, and an ERK inhibitor.
Embodiment 26. The pharmaceutical kit of any one of embodiments 22, 23, or 25, wherein the MEK inhibitor is trametinib, binimetinib, or a combination thereof.
Embodiment 27. The pharmaceutical kit of embodiment 26, wherein the MEK inhibitor is trametinib.
Embodiment 28. The pharmaceutical kit of any one of embodiments 22, 24, or 25, wherein the ERK inhibitor is ulixertinib, ravoxertinib, temuterkib, rineterkib, or a combination thereof.
Embodiment 29. The pharmaceutical kit of any one of embodiments 22-25, wherein the BRAF inhibitor is encorafenib, vemurafenib, or dabrafenib, or a combination thereof.
Embodiment 30. The pharmaceutical kit of embodiment 29, wherein the BRAF inhibitor is vemurafenib.
Embodiment 31. The pharmaceutical kit of any one of embodiments 21-30, wherein the COX-2 inhibitor is celecoxib, valdecoxib or a combination thereof.
Embodiment 32. The pharmaceutical kit of embodiment 31, wherein the COX-2 inhibitor is celecoxib.
Embodiment 33. The pharmaceutical kit of any one of embodiments 21-32, further comprising an epidermal growth factor receptor (EGFR) inhibitor.
Embodiment 34. The pharmaceutical kit of embodiment 33, wherein the EGFR inhibitor is panitumumab, cetuximab, or gefitinib.
Embodiment 35. The pharmaceutical kit of any one of embodiments 21-34, further comprising a SRC inhibitor.
Embodiment 36. The pharmaceutical kit of embodiment 35, wherein the SRC inhibitor is dasatinib, saracatinib, bosutinib or a combination thereof.
Embodiment 37. The pharmaceutical kit of any one of embodiments 21-36, further comprising instructions for treatment of a BRAF intrinsically resistant cancer.
Embodiment 38. The pharmaceutical kit of embodiment 37, wherein the BRAF intrinsically resistant cancer is colorectal cancer, thyroid cancer, glioblastoma, sarcoma, and hepto-sarcoma.
Embodiment 39. The pharmaceutical kit of embodiment 38, wherein the BRAF intrinsically resistant cancer is colorectal cancer, thyroid cancer, glioblastoma, sarcoma, or hepto-sarcoma
Embodiment 40. The pharmaceutical kit of embodiment 39, wherein the BRAF intrinsically resistant cancer is colorectal cancer.

### EXAMPLES

### Example 1: a Reversible SRC-Relayed COX-2 Inflammatory Program Drives Therapeutic Resistance in BRAF^{V600E} Colorectal Tumors

### INTRODUCTION

BRAF^{V600E} mutation confers a poor prognosis in metastatic colorectal cancer (CRC) despite combinatorial targeted therapies based on the latest understanding of signaling circuitry. To identify parallel resistance mechanisms induced by BRAF/MEK/EGFR co-targeting, we used a high throughput kinase activity mapping platform. High-throughput kinase-activity mapping (HT-KAM) is a functional proteomic screening technology which enables direct measurement of the catalytic activity of many kinases in parallel⁷⁻¹⁰. This systematic process can help identify the most significantly and specifically perturbed kinase hubs, in turn revealing actionable vulnerabilities (kinases or otherwise) that lie within phospho-circuits of cancer cells and tissues. Strategic kinase dependencies with the highest therapeutic potential can then be chosen for further investigation in cell culture and xenograft models. The ultimate goal is to identify rational therapeutic combinations capable of producing greater than incremental improvements in clinical outcomes for patients with BRAF^{V600E} mCRC.

We found that a highly conserved SRC-relayed inflammatory program drives the adaptive response to targeted therapies in BRAF^{V600E} CRC. Specifically, SRC family kinases were activated upon treatment with BRAF±MEK±EGFR inhibitors in vitro and in vivo, thus uncovering an EGFR-independent mechanism of resistance. We found that upon treatment with BRAF±EGFR targeted therapy, the activation of SRC kinases regulates the downstream phosphorylation of beta-catenin (CTNNB1), which leads to the reprogramming of cells' transcriptional profiles. Upstream of SRC, we found that SRC kinases were activated by an autocrine prostaglandin E₂ (PGE2)-regulated GNAS-activation loop that COX2 inhibitors interrupted in both cell lines and patient derived xenograft (PDX) mouse models^{4,11}. This SRC-relayed mechanism of therapeutic resistance operated independently of ERK-signaling. We showed that supplementing the current standard-of-care combination of BRAF-inhibitor encorafenib plus an anti-EGFR antibody (panitumumab) with the FDA-approved COX2-inhibitor, celecoxib, significantly and consistently improved tumor growth inhibition. Overall, our study demonstrates that SRC signaling is at the nexus of a cell-autonomous inflammatory program with pro-tumorigenic activities, which explains why BRAF^{V600E} colorectal tumors develop resistance to BRAF/MEK and EGFR inhibitors. Our results demonstrate a new clinically actionable strategy, the addition of celecoxib to targeted therapies, to restore therapeutic response in BRAF^{V600E} CRC. This drug-repurposing approach is cost-effective with minimal added toxicity, and may be fast-tracked into clinical testing.

### Results

### Kinome Screening Identifies SRC Family Kinases as Independent Functional Determinants of the Adaptive Response to BRAF/MEK/EGFR Targeting in BRAF^{V600E} CRC.

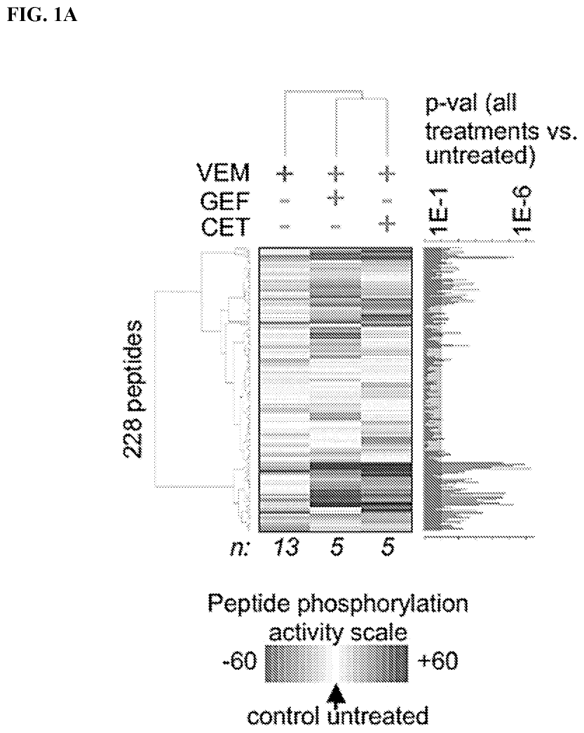

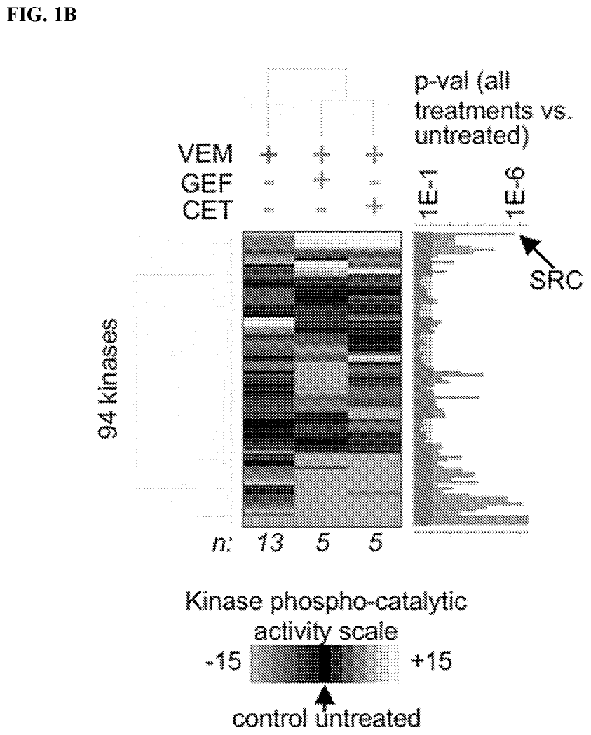

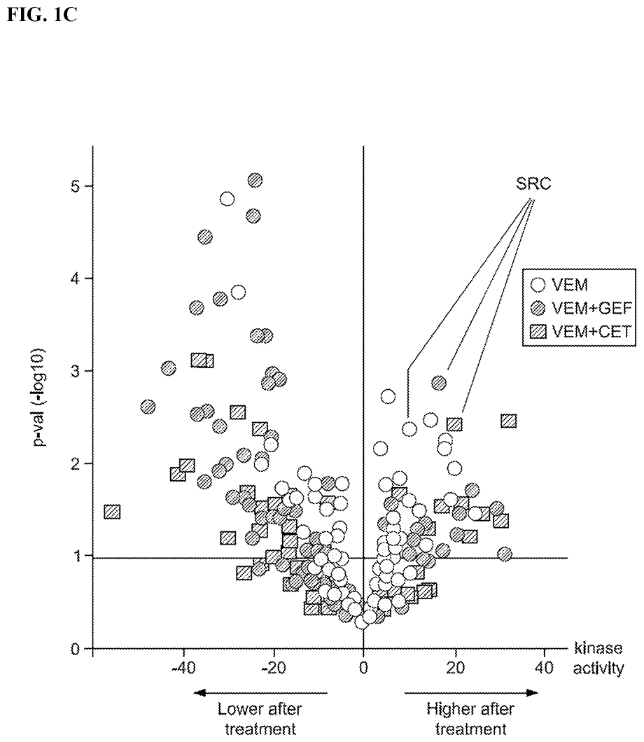

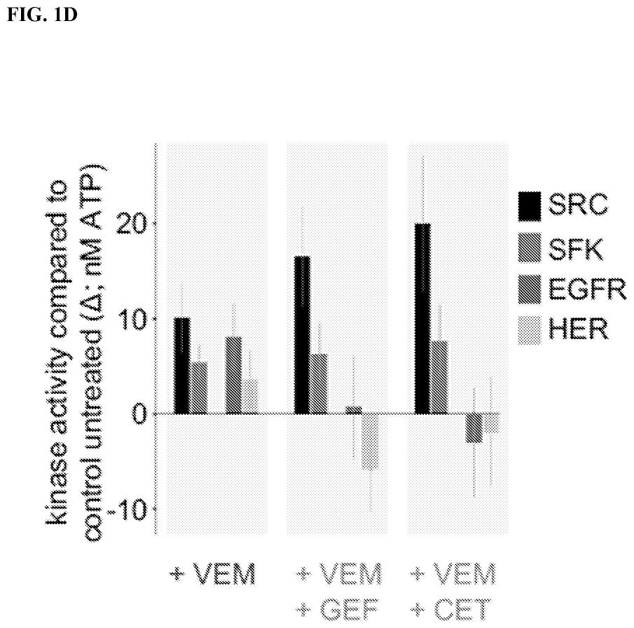

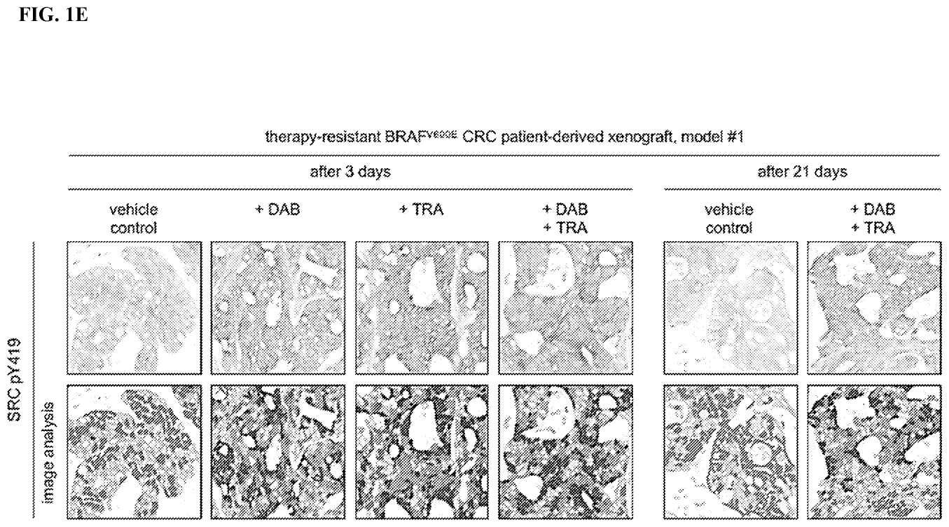

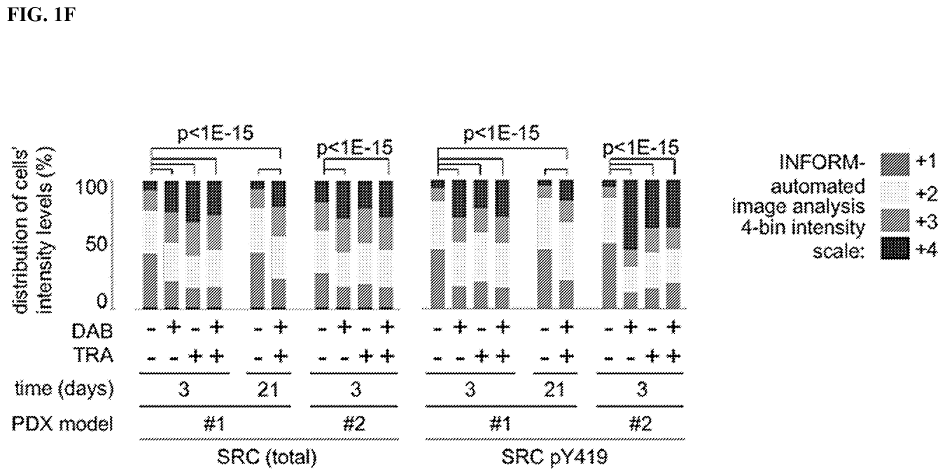

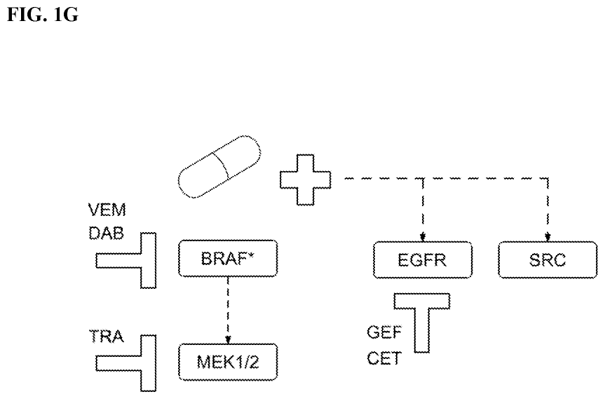

We used the HT-KAM platform to measure kinase activity in extracts processed from WiDr cells, a well-established vemurafenib-resistant BRAF^{V600E} CRC model¹²⁻¹⁴. Cells were treated with a BRAF inhibitor (vemurafenib)±an EGFR inhibitor (gefitinib or cetuximab) for 8 hours. Peptide-level data (FIG. 1A) were then transformed into kinase activity signatures (FIG. 1B) using previously described deconvolution methods⁷. We found that SRC displayed the most significantly increased kinase activity in response to BRAF inhibitor containing treatments (FIG. 1B). Remarkably, the increase in SRC activity was conserved even following combined treatment with vemurafenib and either EGFR inhibitors (FIG. 1B-1D), indicating that SRC is not a surrogate for EGFR-mediated resistance to BRAF targeted therapies.

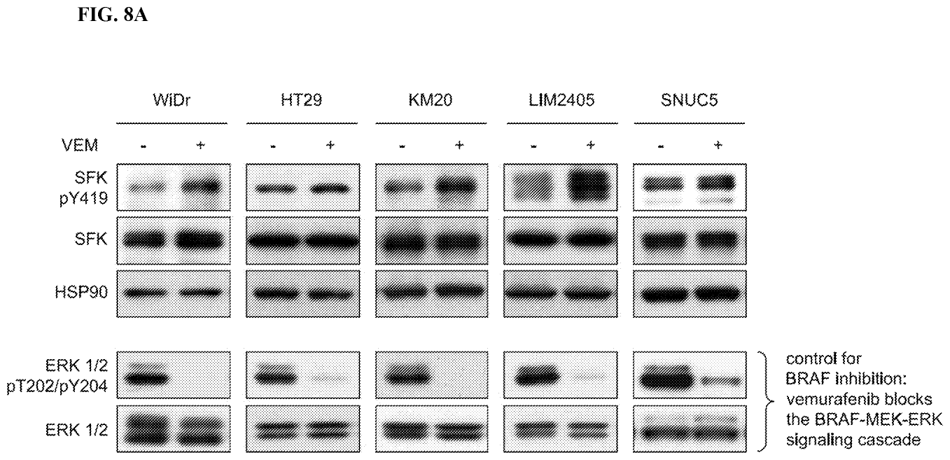

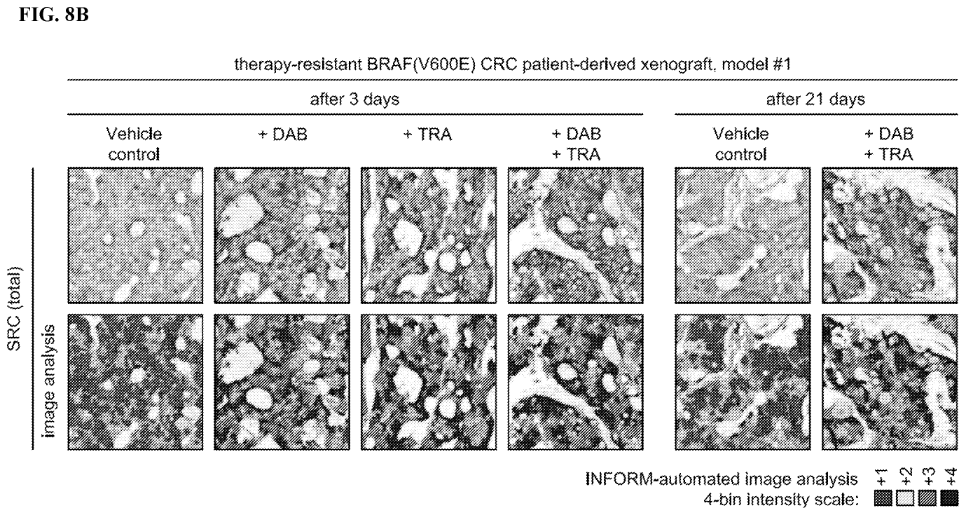

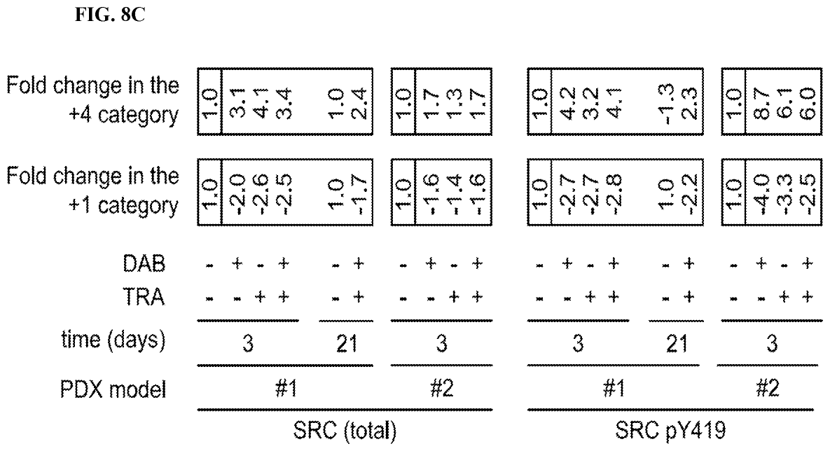

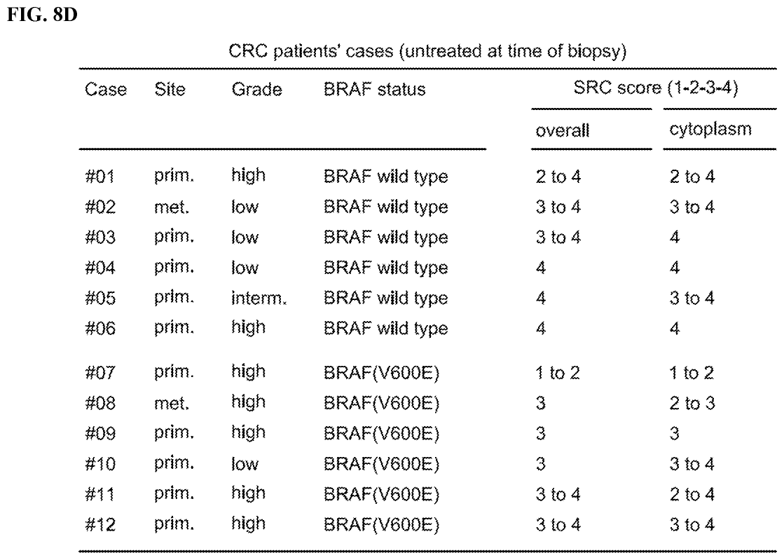

SRC belongs to the SRC family kinase (SFK), which is composed of 11 membrane-associated, non-receptor tyrosine kinases that regulate cell proliferation, differentiation, apoptosis, migration, and metabolism among other processes¹⁵⁻¹⁸. We validated the observed kinase activity signature by western blot in a panel of BRAF^{V600E} CRC lines after vemurafenib treatment. Results show increased SFK activation, as reported by the phosphorylation of Y419 (FIG. 8A). We sought further substantiation of SRC-mediated resistance using rare xenograft models of BRAF^{V600E} CRC derived from patients who were subsequently treated with dabrafenib and trametinib on a clinical trial 4. PDX #1, and the corresponding patient's tumor biopsy, exhibited primary resistance to treatment with dabrafenib+trametinib; whereas patient/PDX #2 exhibited early tumor regression and eventual progression. Using automated image analysis of IHC profiles, we observed statistically significant increases in active and total SRC in both PDX models after 3 or 21 days of treatment with dabrafenib and/or trametinib (FIGS. 1E, 1F and FIG. 8B, 8C). These data place SRC activation as an early adaptive response to BRAF inhibition, which is maintained even in residual tumors following BRAF and/or MEK inhibitor treatment. Unlike EGFR levels that were upregulated at baseline in BRAF^{V600E} cell lines^{12,13}, comparison of untreated primary patient colorectal tumor specimens harboring or not a BRAF^{V600E} mutation showed that patient tumors start with similar levels of total SRC (FIG. 8D). Together these findings led us to postulate that SRC is an EGFR-independent candidate drug target to overcome resistance to BRAF/MEK inhibitor therapies (FIG. 1G).

### SRC Kinase Inhibitors Sensitize BRAF^{V600E} CRC Cells to Vemurafenib.

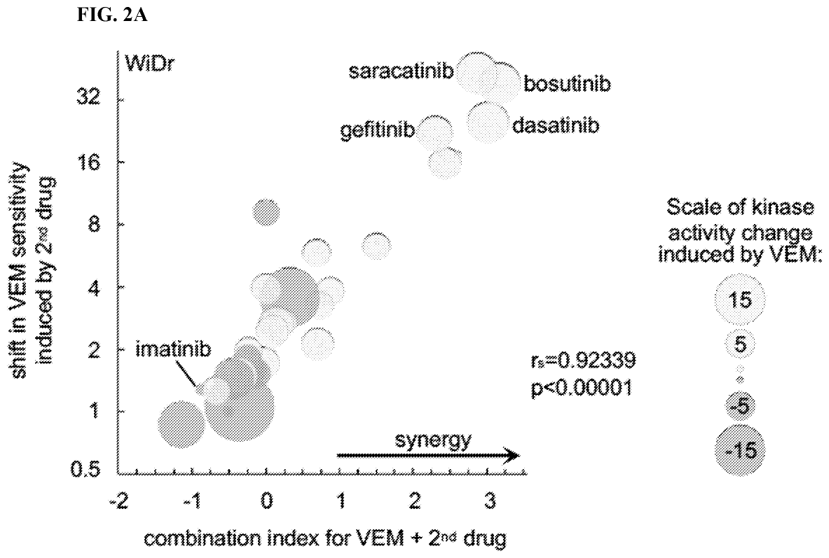

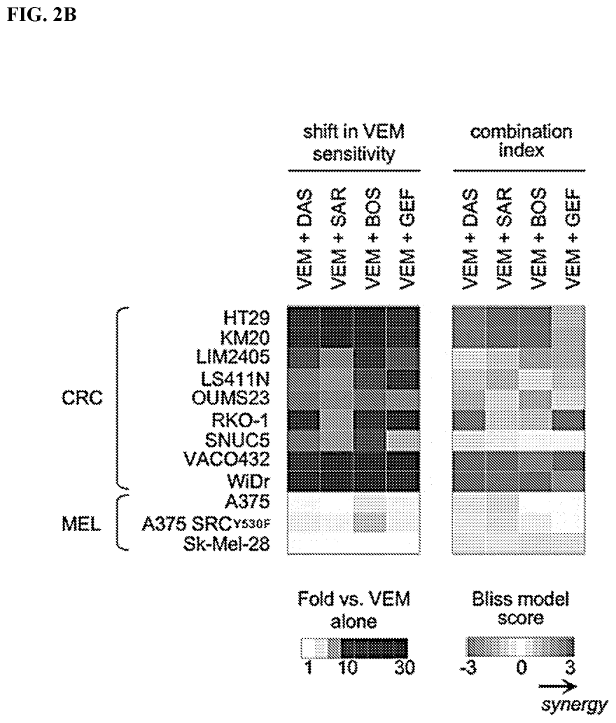

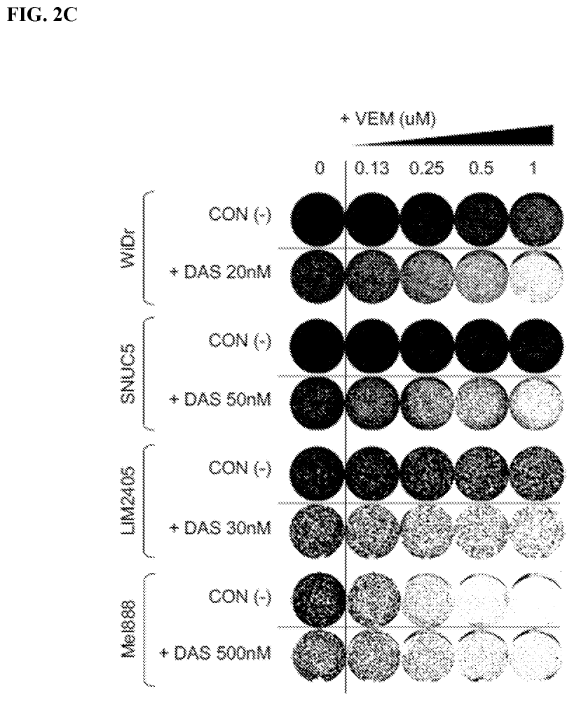

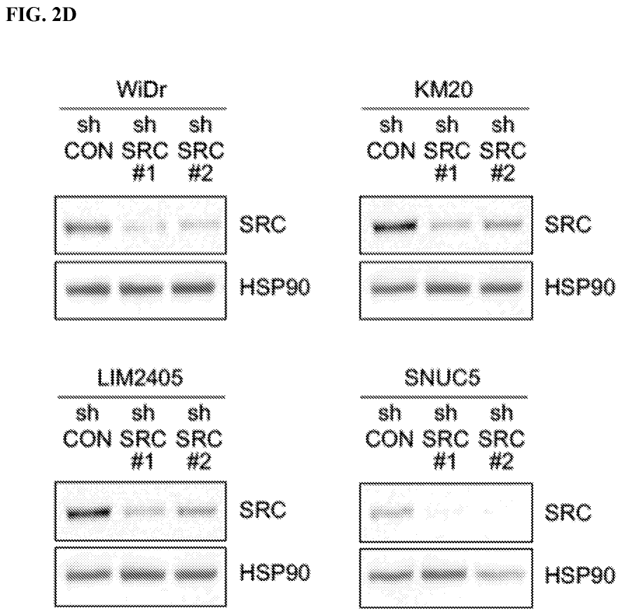

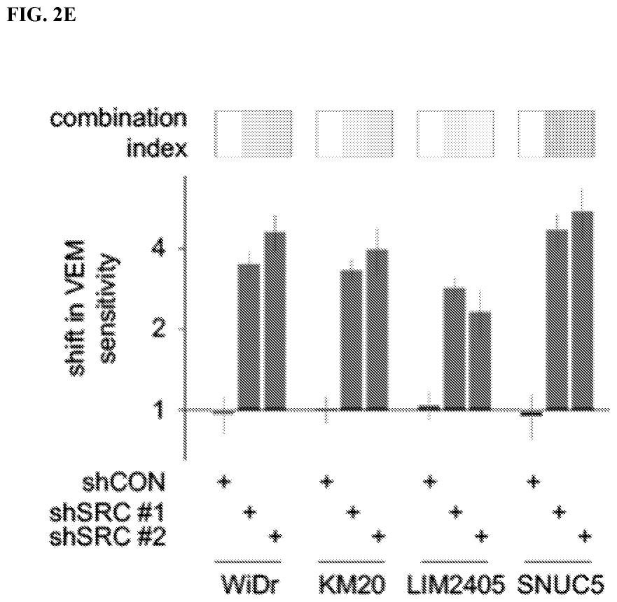

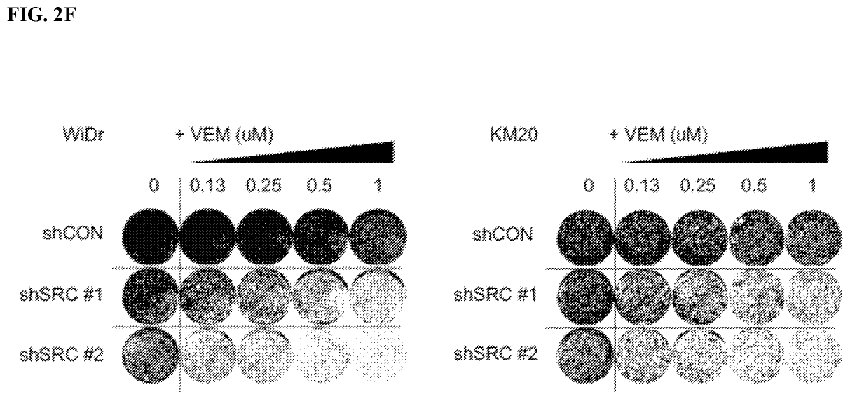

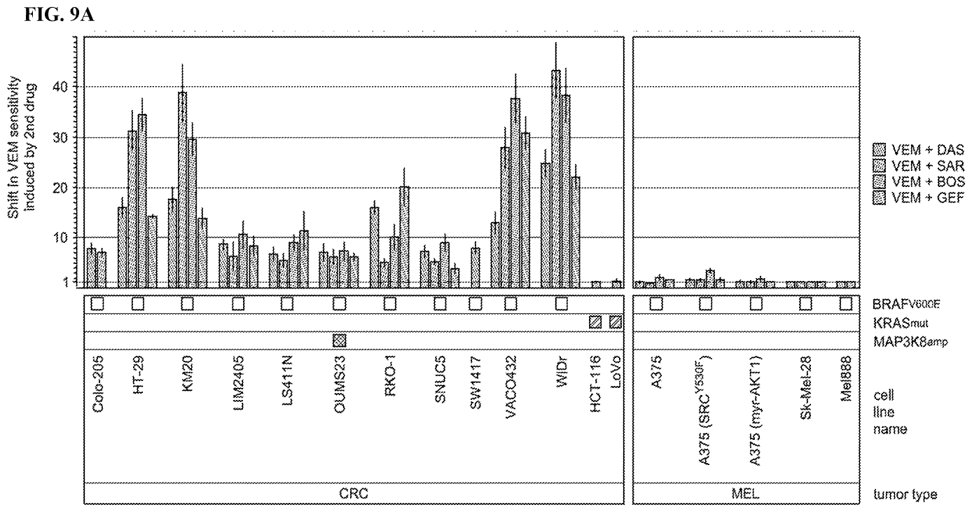

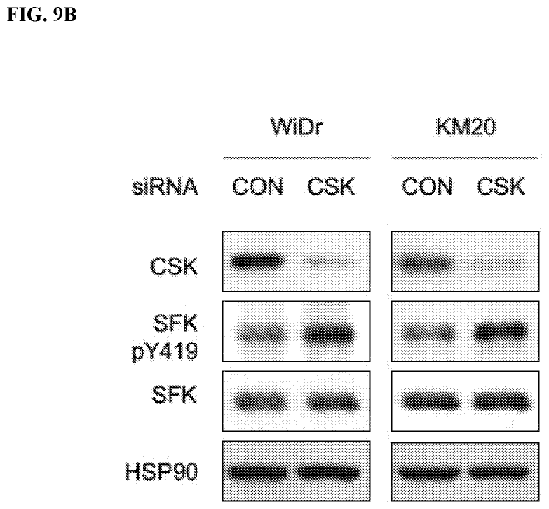

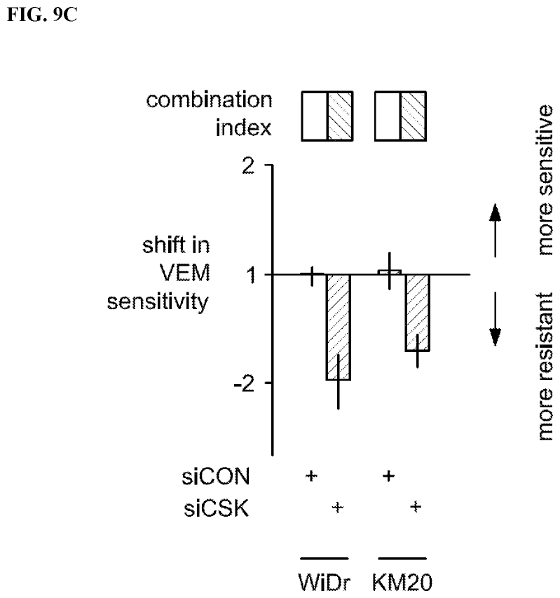

To test the hypothesis that SRC is a druggable vulnerability in BRAF inhibitor resistant BRAF^{V600E} CRC, we first assessed the sensitivity of WiDr cells to two-drug combinations including a BRAF inhibitor and another kinase inhibitor, chosen based on the kinase signatures in FIG. 1B. Consistently, the greatest increase in cell growth inhibition and the highest combination index (CI) scores, i.e. synergy, were observed when the BRAF inhibitor was combined with a SRC inhibitor: dasatinib, saracatinib, or bosutinib (FIG. 2A; all CI>2.8). The potentiation in sensitivity to vemurafenib with the addition of a SRC inhibitor was conserved across various vemurafenib-resistant BRAF^{V600E} CRC cell lines (HT29, KM20, LIM2405, LS411N, OUMS23, RKO1, SNUC5, VAC0432, WiDr), as shown in FIG. 2B; the EGFR-inhibitor gefitinib is included for comparison. This observation is specific to BRAF^{V600E} CRC, as it was not recapitulated in vemurafenib-sensitive BRAF^{V600E} melanoma cells (A375, A375 (SRC^{Y530F}), A375 (myr-AKT1), Sk-Mel-28, Mel888) or BRAF wild-type CRC cells (HCT116, LoVo), as shown in FIG. 2B and FIG. 9A. Furthermore, dual treatment with vemurafenib+dasatinib strongly inhibited colony formation in BRAF^{V600E} CRC cells, but not BRAF^{V600E} melanoma cells, at concentrations where dasatinib alone has a minimal effect (FIG. 2C). To further validate SRC's role as a mediator of response to vemurafenib, we knocked down SRC using short hairpin RNA (shRNA) (FIG. 2D) and found that SRC-deficient BRAF^{V600E} CRC cells were more sensitive to vemurafenib treatment in 3-day viability and colony formation assays (FIG. 2E, 2F), although as expected not as profoundly as when using SFK inhibitors. Conversely, small interfering RNA (siRNA) knockdown of C-terminal SRC kinase (CSK), a negative regulator of SFK, led to increased SFK activation (FIG. 9B) and reduced sensitivity to vemurafenib (FIG. 9C). Collectively these data substantiate our hypothesis that SRC is a promising new target to overcome resistance to BRAF inhibitor therapies in BRAF^{V600E} CRC.

### SRC Inhibition Systematically Improves Efficacy of BRAF+EGFR Targeting in BRAF^{V600E} CRC In Vitro and In Vivo.

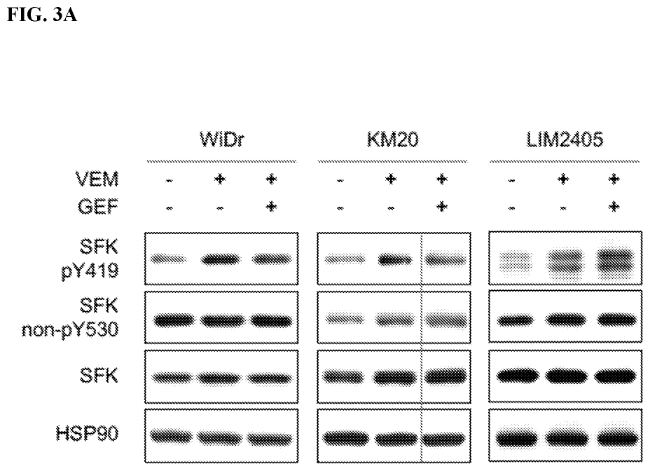

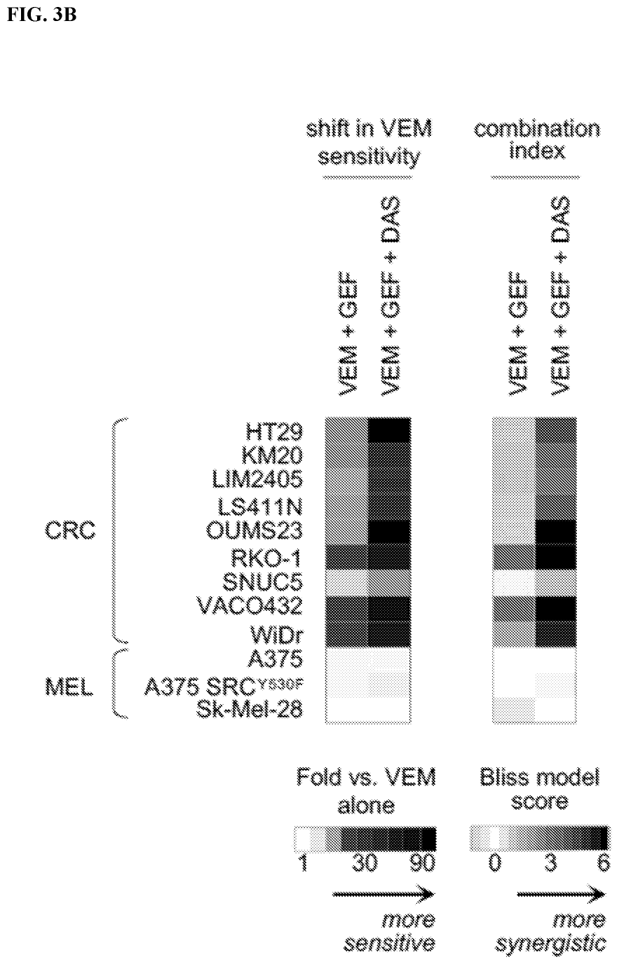

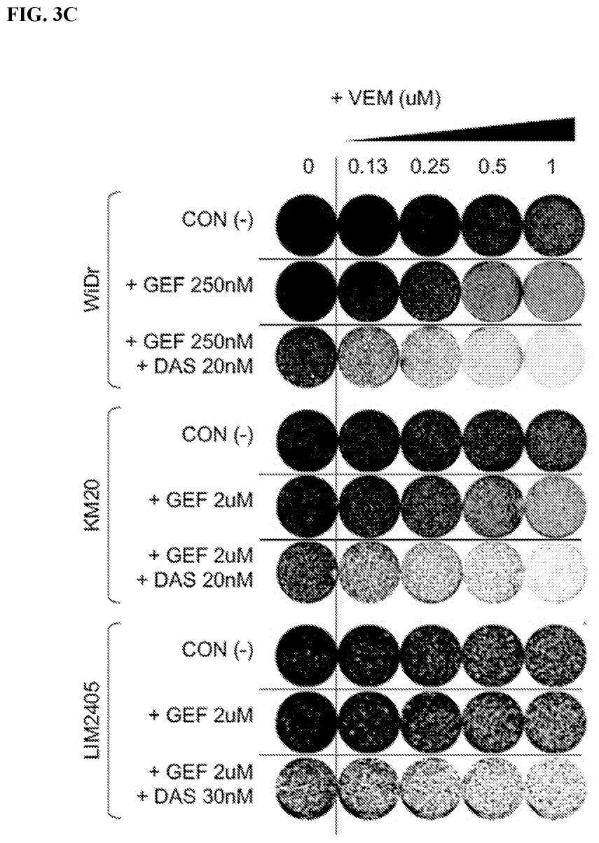

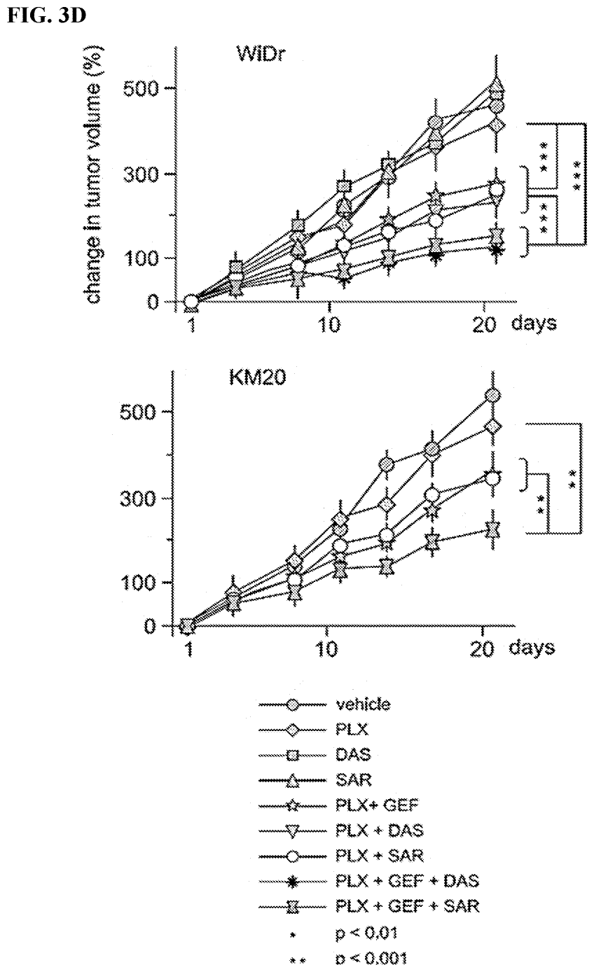

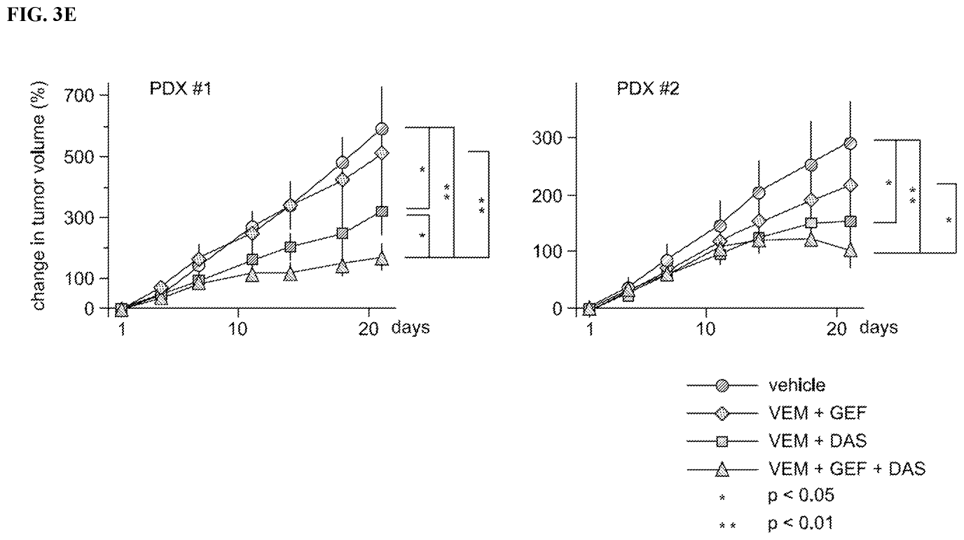

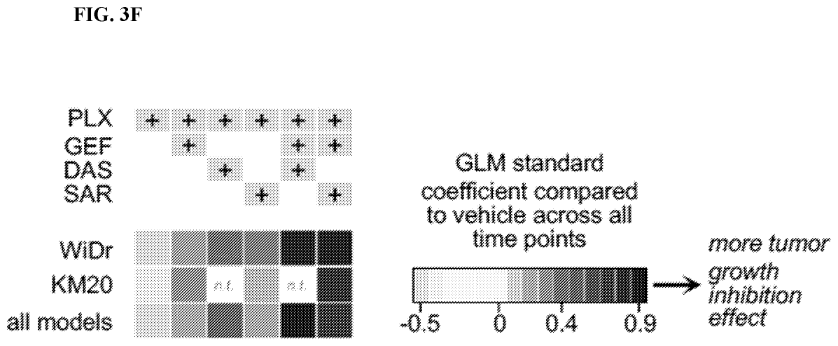

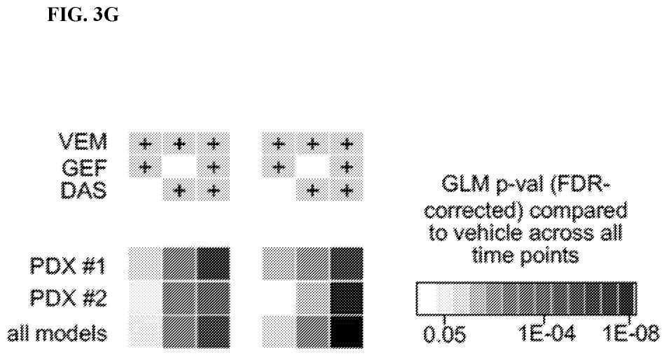

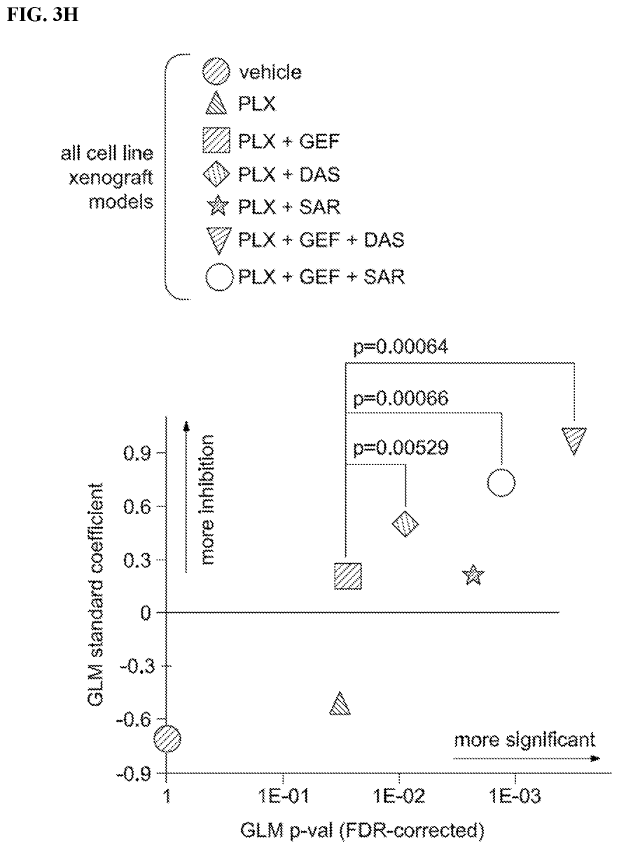

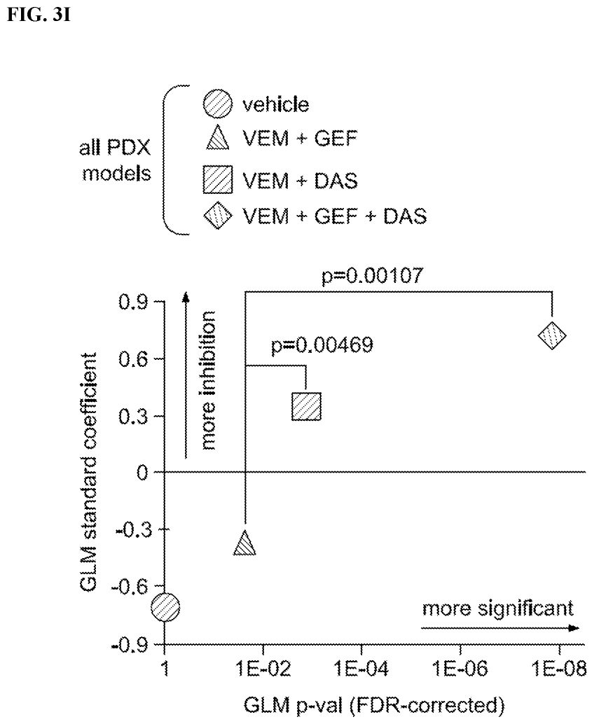

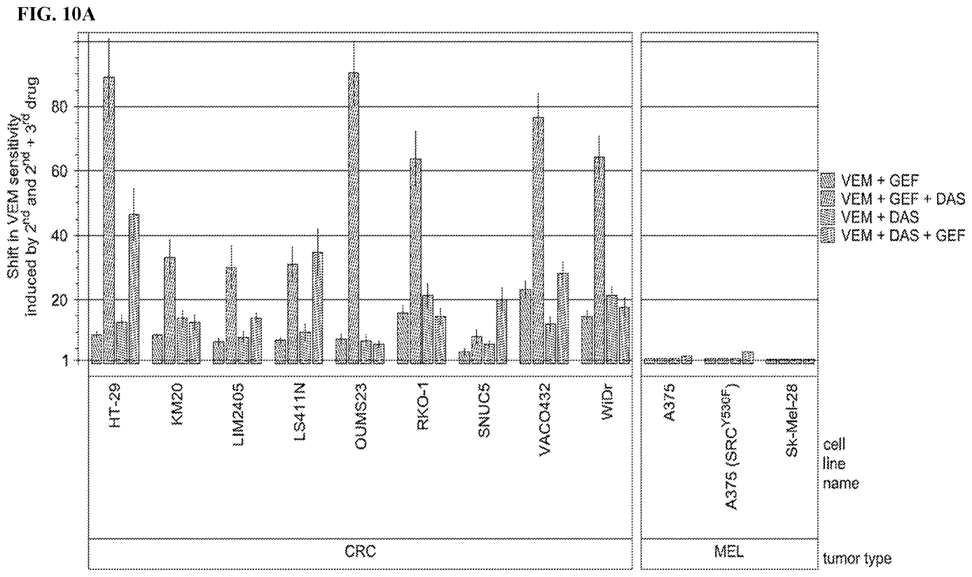

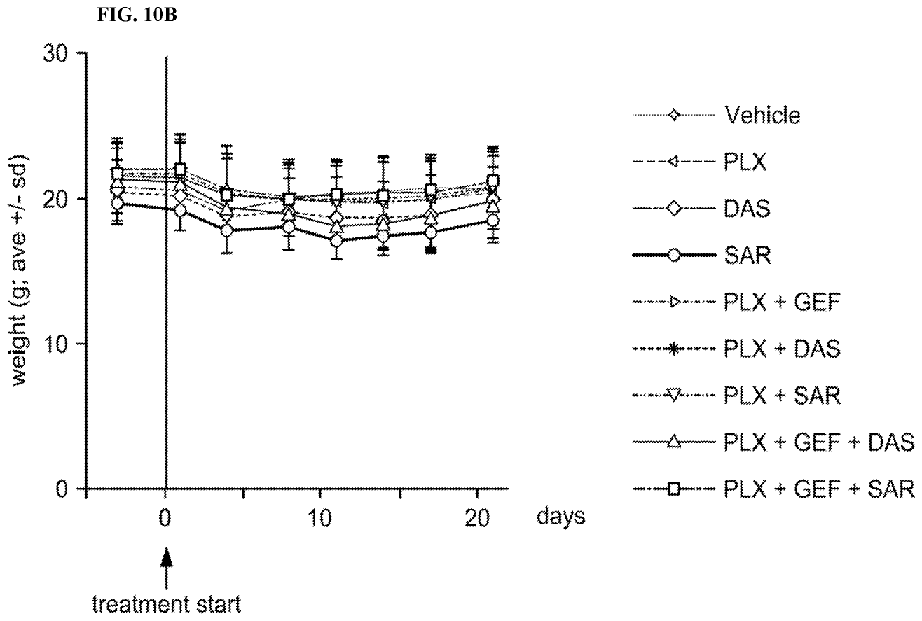

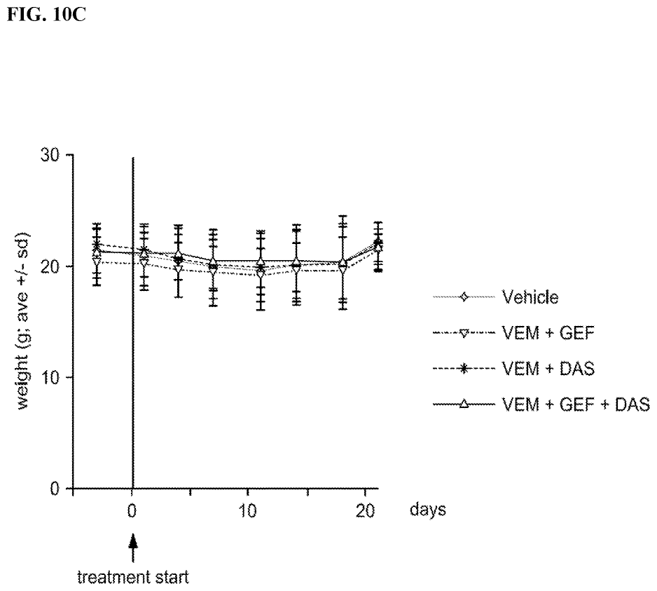

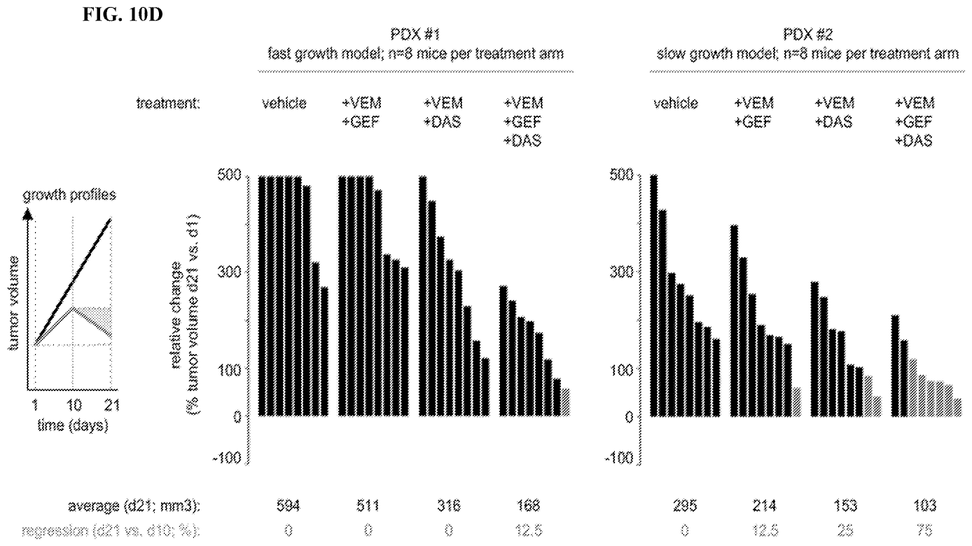

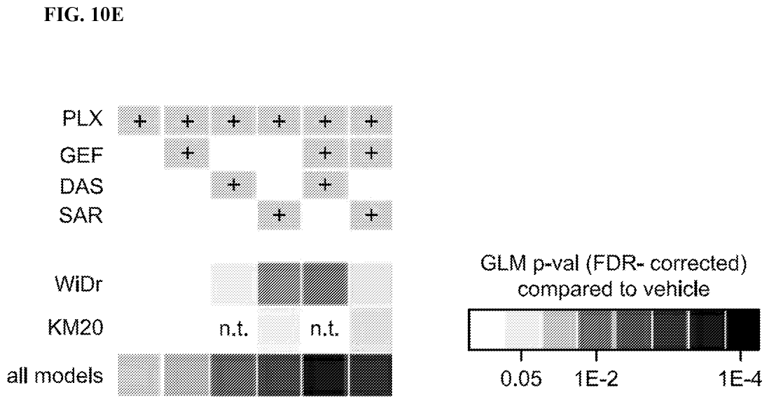

Since a BRAF+EGFR inhibitor doublet is the first FDA approved molecularly targeted regimen for patients with BRAF^{V600E} mCRC 5, we next asked whether the efficacy of BRAF and EGFR targeting can be improved by addition of a SRC inhibitor. To begin, we verified SFK activation after vemurafenib+gefitinib treatment in a panel of BRAF^{V600E} CRC cell lines (FIG. 3A). The analysis showed increased SFK activation reflected by increased Y419 phosphorylation and Y530 de-phosphorylation, which further substantiates findings using HT-KAM in FIGS. 1B-1D. Moreover, triplet therapy with the addition of dasatinib to vemurafenib and gefitinib resulted in a synergistic increase in sensitivity to vemurafenib, greater than which was observed for either vemurafenib-containing doublet (FIG. 3B and FIG. 10A). The synergistic impact on cell viability with the addition of a SRC inhibitor to BRAF+EGFR targeting was conserved across BRAF^{V600E} CRC cell lines, but not BRAF^{V600E} melanoma cells. Likewise, triplet treatment with vemurafenib plus gefitinib and dasatinib more effectively inhibited colony formation in BRAF^{V600E} CRC cells as compared to vemurafenib+gefitinib (FIG. 3C). Encouraged by these data, we tested combinations of BRAF±EGFR and SRC inhibitors first in cell line-derived xenografts (FIG. 3D) and then in the same PDX models evaluated in FIG. 1 (FIG. 3E). In all four xenograft models, triplet combinations resulted in statistically significantly improved tumor growth inhibition as compared to BRAF+EGFR or BRAF+SRC inhibitor doublets. Toxicity, as assessed by mouse weight and distress, was negligible (FIG. 10B, 10C). Moreover, tumor regression was observed beyond the mid-point of treatment in multiple PDX tumors treated with dasatinib-containing regimens (FIG. 10D). Next, we used a Generalized Linear Model (GLM) to quantify the effects of drug combinations vs. vehicle over time (FIGS. 3F-3I). Based on results in FIGS. 3D-3F we found that the addition of a SRCi to a BRAFi had an equivalent or better effect on tumor growth inhibition in comparison to adding an EGFRi to a BRAFi. Furthermore, triple therapy with BRAFi+EGFRi+SRCi significantly improved tumor growth inhibition compared to any doublet (FIGS. 3F-3G; GLM standard coefficients>0.8 and >0.5 for cell line xenografts and PDXs respectively; FDR-corrected p-val in FIG. 10E and in the right panel of FIG. 3G). FIGS. 3H, 3I highlight that the addition of a SRCi to a BRAFi+EGFRi systematically and significantly improves effect size in both cell line and PDX models.

### The Activation of SRC Upon BRAF±EGFR Inhibition Regulates Beta-Catenin Transcriptional Reprogramming of BRAF^{V600E} CRC Cells.

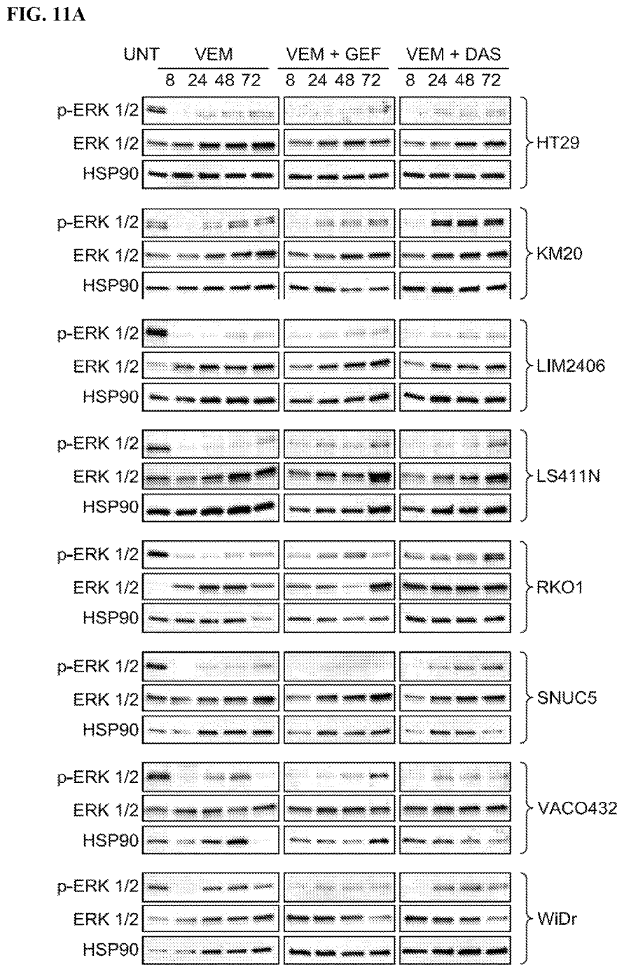

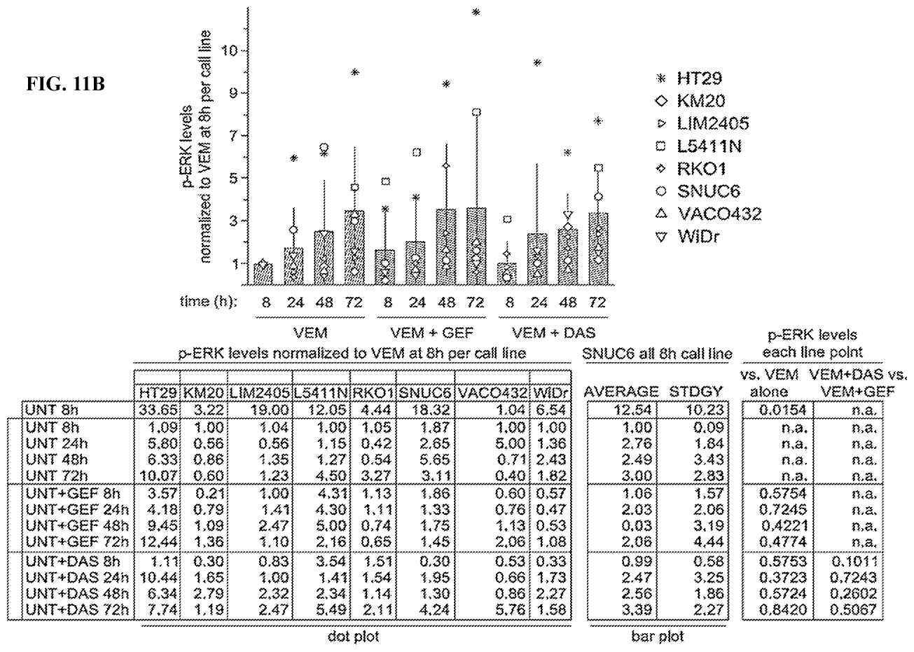

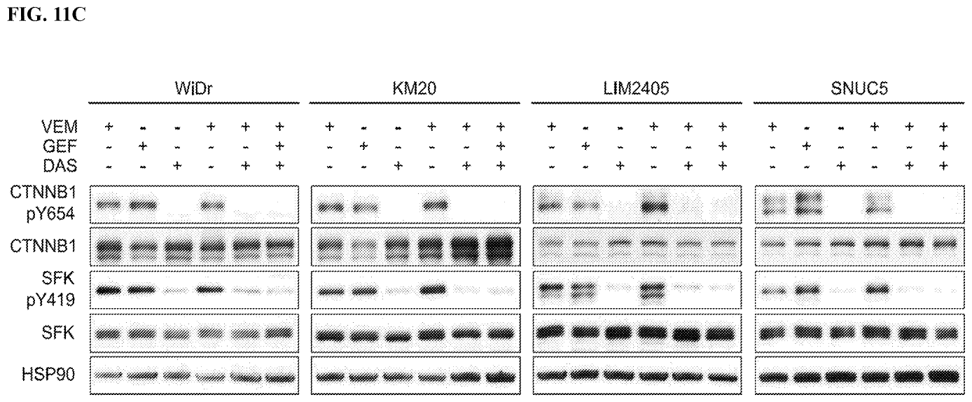

Next, we sought to elucidate the mechanism underlying the synergistic effects of co-targeting SRC and the MAPK pathway±EGFR shown in FIGS. 2A-2E, 3A-3I. MAPK signaling rebound is recognized as an important mechanism of resistance^{12,13}, so we tested whether adding the SRCi dasatinib would inhibit phospho-ERK rebound more profoundly than would BRAFi alone or BRAFi+EGFRi. Using 8 BRAF^{V600E} CRC cell lines collected at 4 different times (up to 72 h) with BRAFi or BRAFi+EGFRi or BRAFi+SRCi, we found that SRC inhibition did not significantly impact rebound of ERK phosphorylation (FIG. 11A, 11B). This suggests that SRC activation in response to BRAF±EGFR targeted therapy treatment acts via a distinct mechanism of drug resistance.

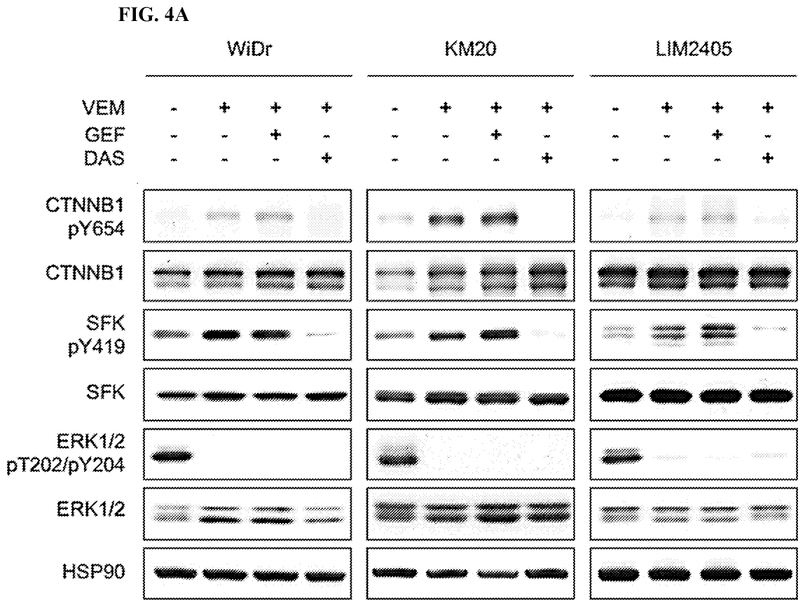

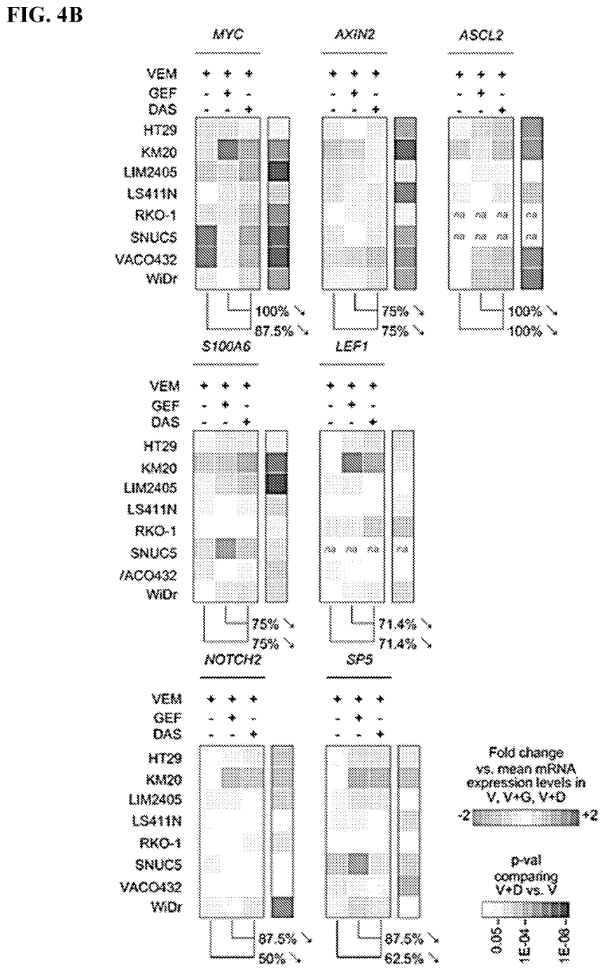

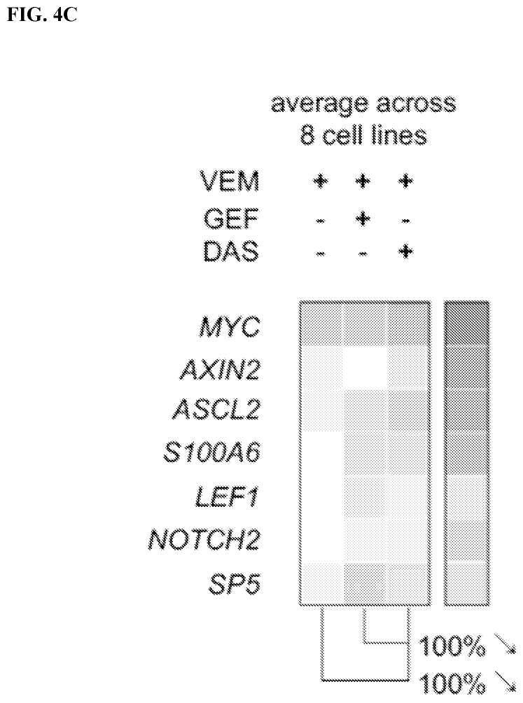

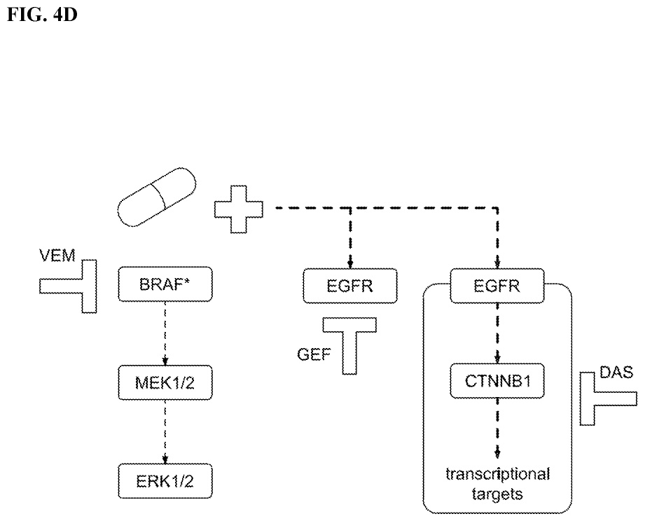

Since many kinases can propagate their pro-oncogenic activities via transcriptional re-programming, we hypothesized that SRC kinases may regulate the phosphorylation state of transcription factors involved in the compensatory response to BRAF±EGFR inhibition. We first used the PhosphoAtlas kinase-substrate interaction database⁸ to identify candidate transcription factor targets of SRC; we then assessed protein phosphorylation profiles by western blot. We found that the phosphorylation of beta-catenin (CTNNB1) at Y654, which is an understudied phospho-target site of SRC kinases⁵⁶, was increased upon BRAFi or BRAFi+EGFRi treatment, but was strongly decreased upon BRAFi+SRCi treatment (FIG. 4A and FIG. 11C). To assess whether these changes in CTNNB1 Y654 phosphorylation impact the transcriptional activities of CTNNB1, we measured the expression levels of a series of CTNNB1 target genes⁵⁷ using quantitative RT-PCR. We found that adding the SRCi dasatinib to BRAFi led to a significant decrease in mRNA levels of all beta-catenin target genes we tested (i.e., MYC, AXIN2, ASCL2, S100A6, LEF1, NOTCH2, SP5) in comparison to BRAFi or BRAFi+EGFRi treated cells (FIGS. 4B, 4C). This indicates that the activation of SRC kinases upon BRAF±EGFR targeting, regulates the function of beta-catenin, a crucial transcription factor in CRC tumorigenesis, which can in turn re-program cells to sustain and adapt to therapeutic pressure. Altogether, SRC activation induces a tumor survival mechanism that acts in parallel to both the MAPK and EGFR signaling axes in BRAF^{V600E} CRC (FIG. 4D).

In BRAF^{V600E} CRC, cVelooxygenase-2 (COX2)/Prostaglandin E2 (PGE2) Upregulation Drives BRAF Inhibitor-Induced SRC Activation In Vitro and In Vivo.

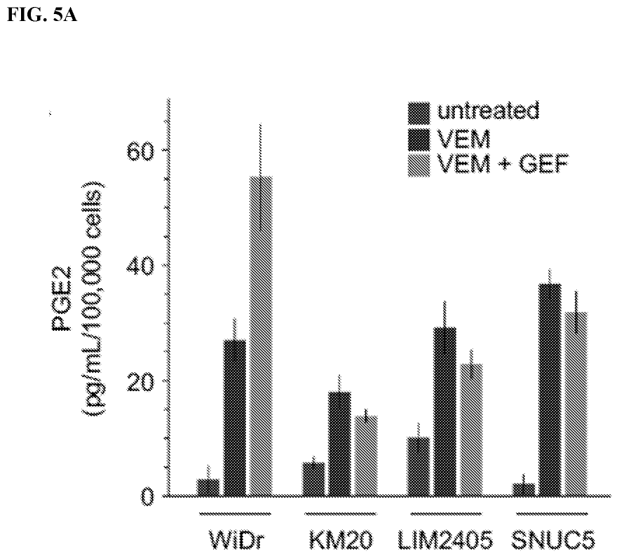

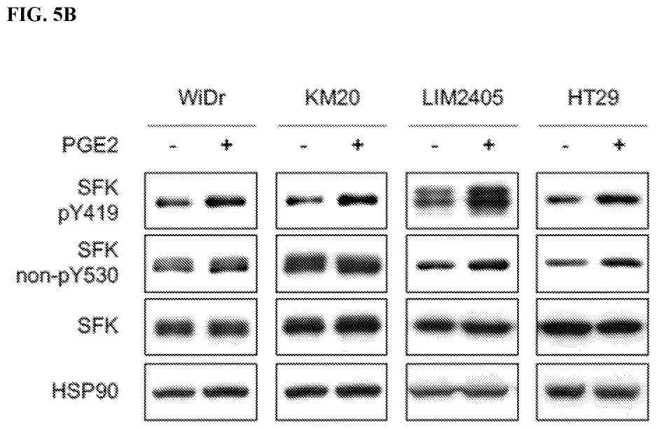

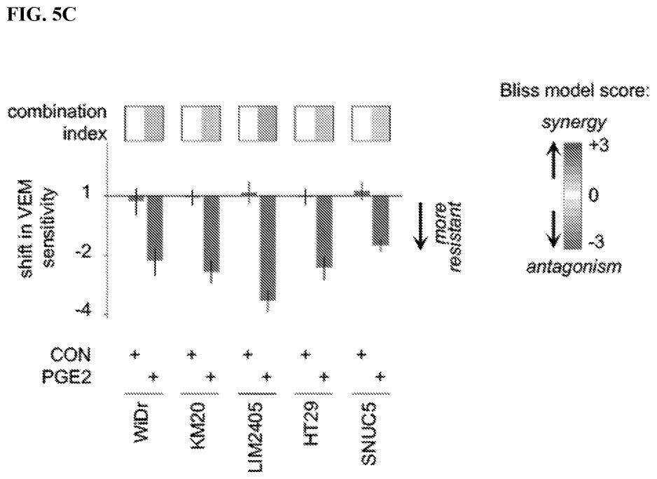

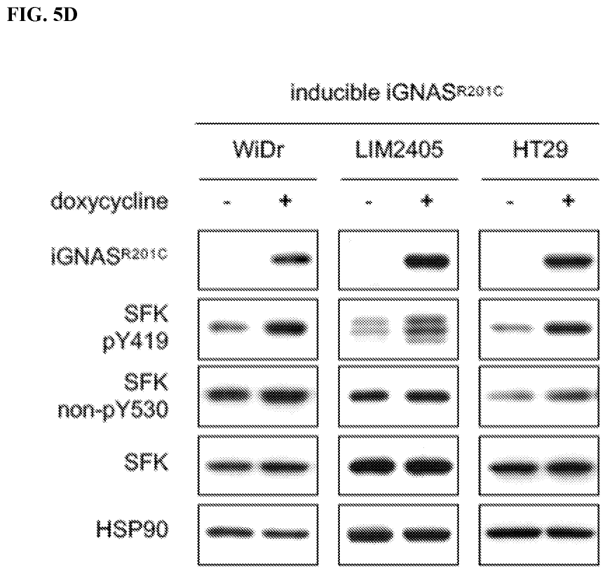

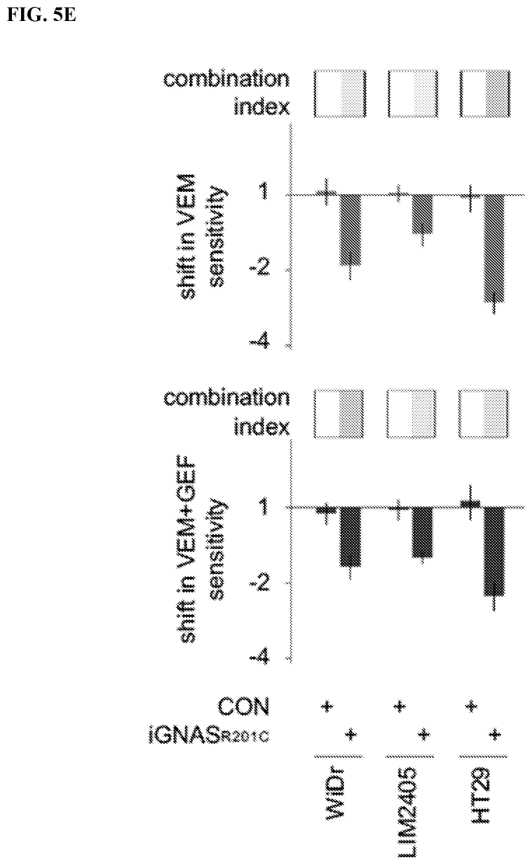

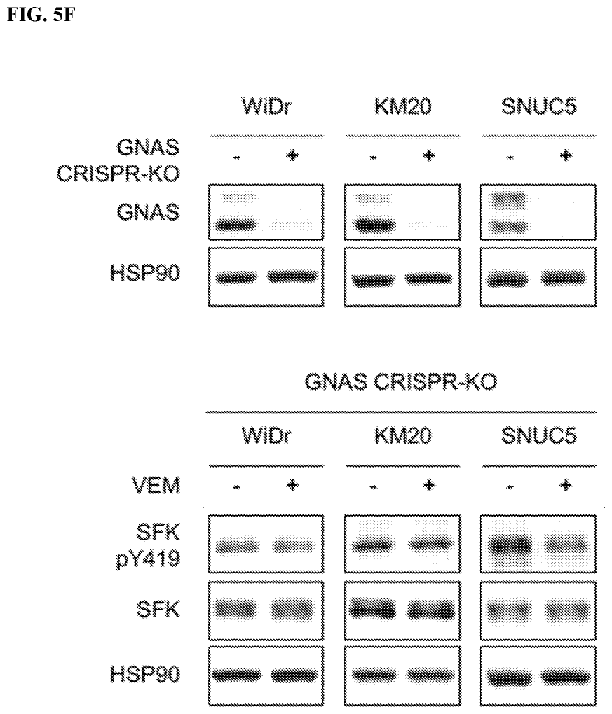

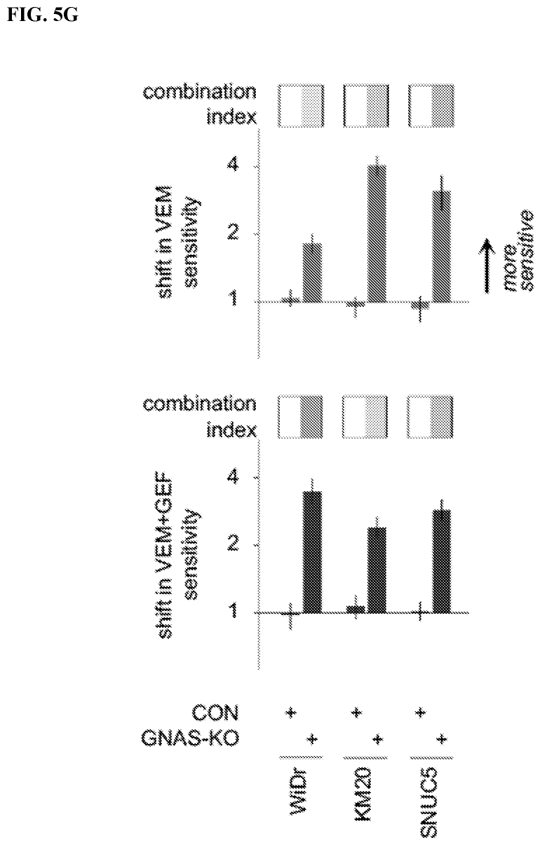

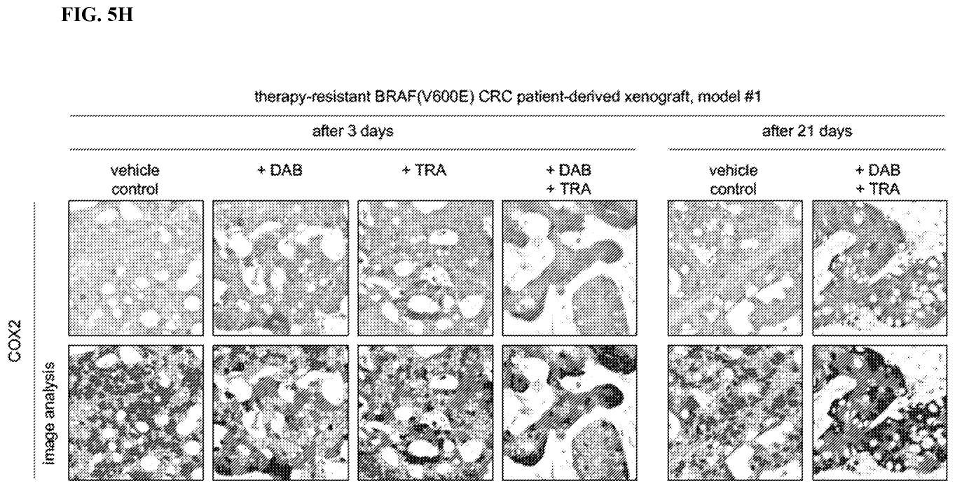

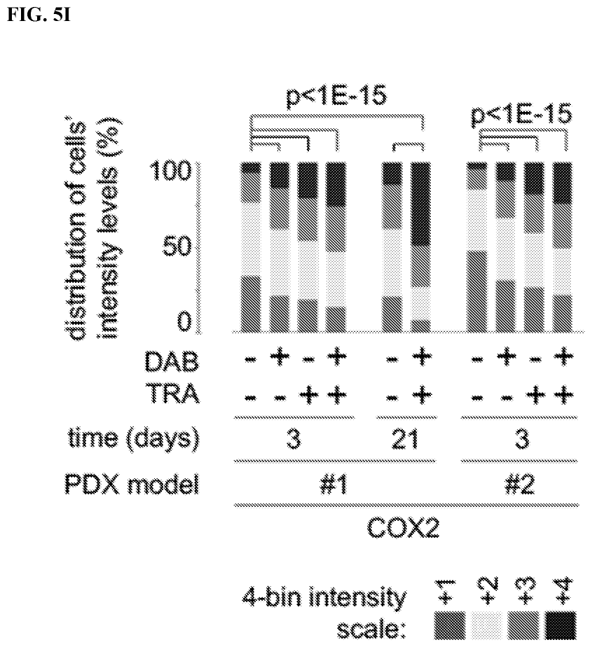

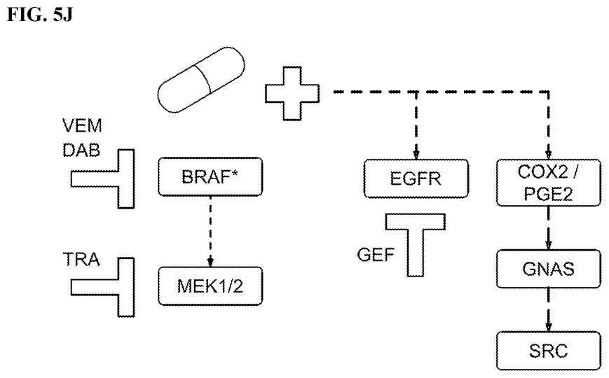

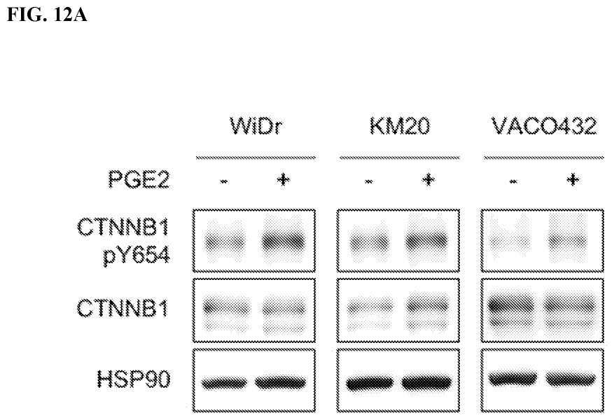

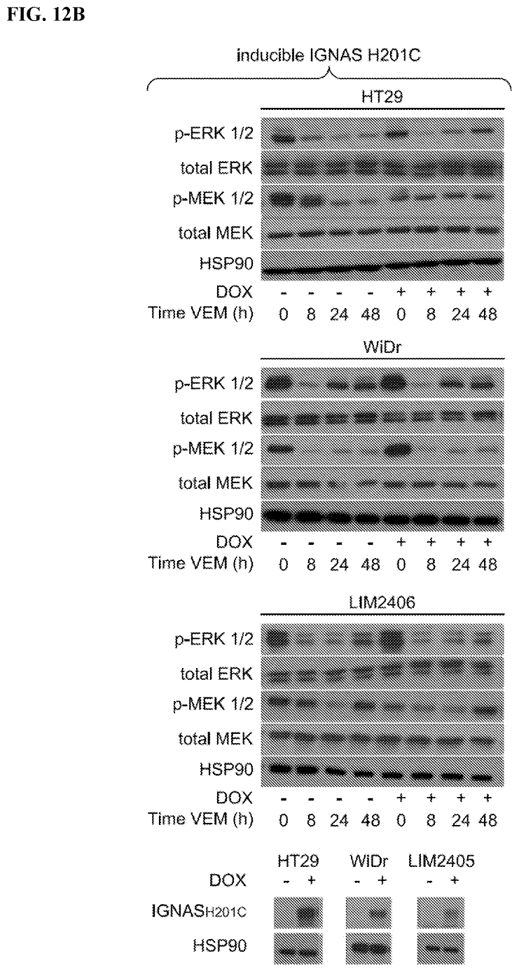

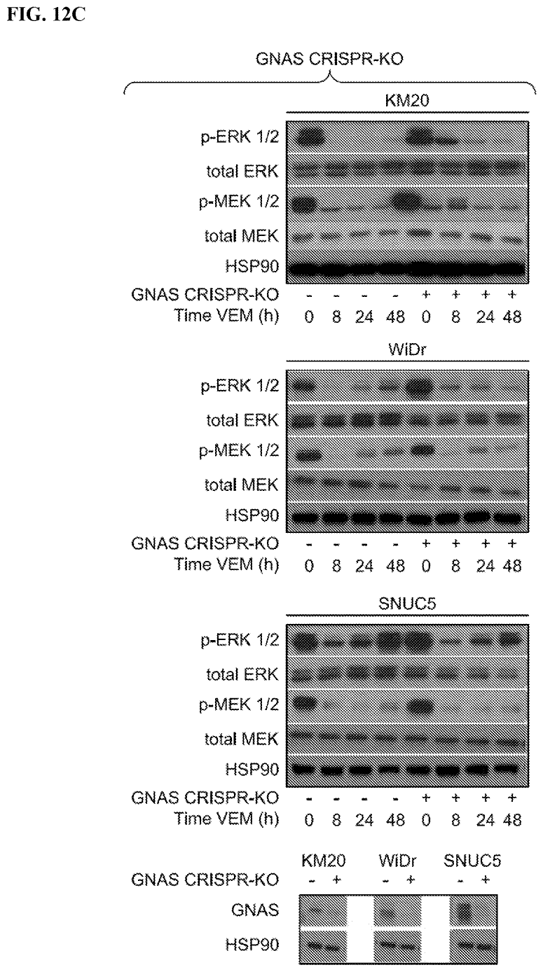

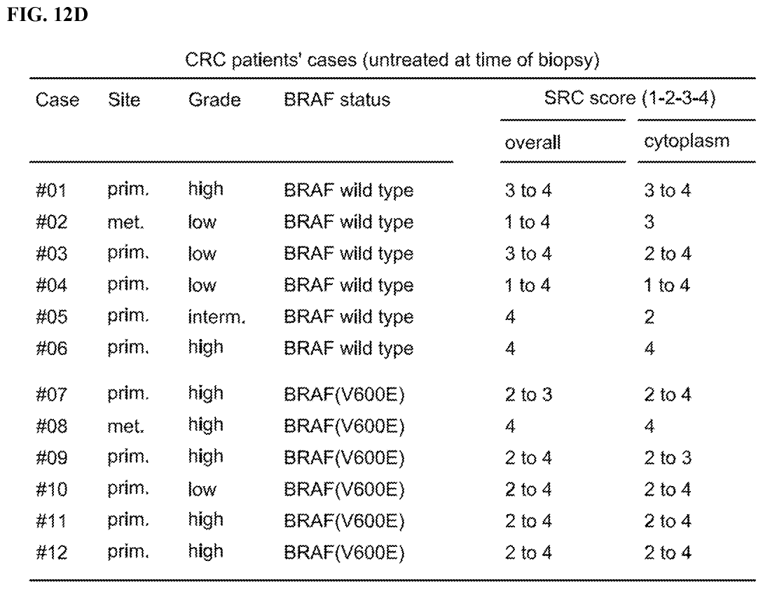

We next asked whether characterization of upstream effectors of SFK could lead to a more clinically appropriate regimen. It has been reported previously that prostaglandin E2 (PGE2) can induce activation of SRC in CRC cells, without specific attention to BRAF^{V600E} mutation status^{19, 20} Using a panel of BRAF^{V600E} CRC cell lines, an increase in PGE2 levels in the media was consistently found as a result of BRAF±EGFR inhibition (FIG. 5A). Treatment of BRAF^{V600E} CRC cells with PGE2 led to an increase in SFK activation (FIG. 5B), and a 2 to 4-fold increase in resistance to vemurafenib, with CI scores indicating antagonism (FIG. 5C). Consistent with the SRC/beta-Catenin signaling cascade established in FIGS. 4A-4D, we found that PGE2 treatment led to an increase in phosphorylation of CTNNB1 Y654 (FIG. 12A).

High levels of PGE2 promote tumor growth by eliciting aberrant extracellular signaling via its G-protein-coupled receptors (GPCRs), EP2 and EP4, and their key downstream effector, GNAS^{19, 21-23}. To mimic the effect of PGE2, we engineered three BRAF^{V600E} CRC cell lines with doxycycline-inducible expression of a constitutively active GNAS mutant (GNAS^{R201C}) When these cells were treated with doxycycline, GNAS^{R201C} was induced, leading to increased SFK activation (FIG. 5D). Induction of GNAS also rendered the cells more resistant to vemurafenib±gefitinib treatment; CI scores again indicate antagonism (FIG. 5E). On the other hand, suppressing GNAS expression by CRISPR-knock out GNAS in BRAF^{V600E} CRC cell lines (GNAS-KO) prevented SFK activation after vemurafenib treatment (FIG. 5F). GNAS-KO cells displayed increased sensitivity to BRAF±EGFR inhibition, with CI scores showing synergy (FIG. 5G), indicating that treatment-dependent SFK activation in BRAF^{V600E} CRC cells is downstream of PGE2/GNAS signaling. We noted that neither GNAS^{R201C}-induced activation of SFK nor GNAS-CRISPR knockout-induced inhibition of SFK activity impacted the rebound of ERK phosphorylation FIG. 12B, 12C), further indicating that the feedback activation of the PGE2-GNAS-SRC signaling axis acts in concert with the MAPK-cascade to drive therapeutic resistance.

It is well established that PGE2 expression and secretion are regulated by COX2⁵⁸. COX2 upregulation in BRAF^{V600E} CRC PDX tumors treated with dabrafenib+trametinib for 3 or 21 days was corroborated by IHC (FIG. 5H, 5I). Paralleling what was observed with SRC in these same PDX tumors (FIG. 1E), upregulation of COX2 is an early adaptive response to BRAF/MEK inhibition that persists in residual tumors even at late time points. In untreated primary patient colorectal tumor specimens with or without a BRAF^{V600E} mutation, COX2 levels were similar (FIG. 12D), again corresponding to observations with SRC (FIG. 8D). In summary, these findings suggest that, in BRAF^{V600E} CRC, the COX2/SRC/beta-catenin signaling does not overlap with the EGFR/BRAF/MAPK signaling, and it is the treatment with BRAF±MEK or EGFR targeted therapies that triggers the compensatory upregulation of a pre-existing COX2-PGE2-GPCR-GNAS autocrine loop, which in turn activates SRC (FIG. 5J).

### COX2 Inhibition Synergistically Improves Efficacy of BRAF/MEK/EGFR Targeting in BRAF^{V600E} CRC In Vitro and In Vivo.

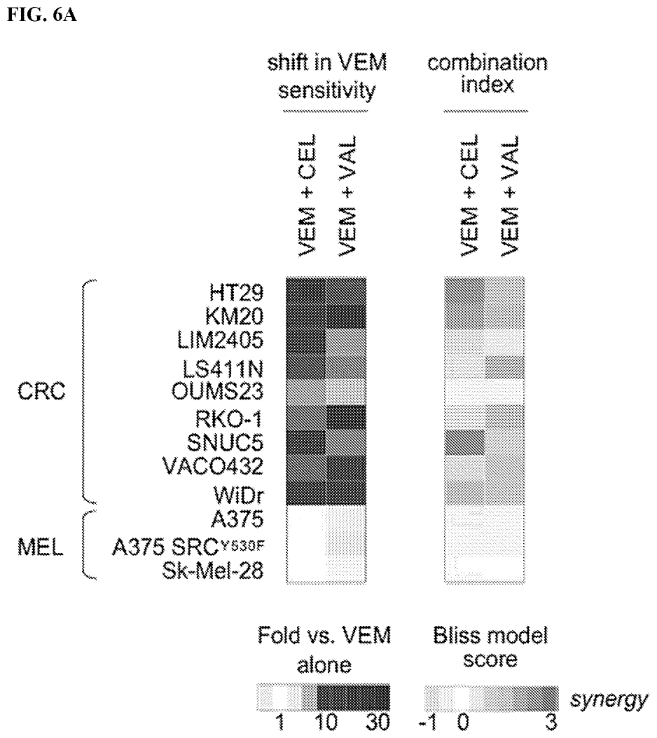

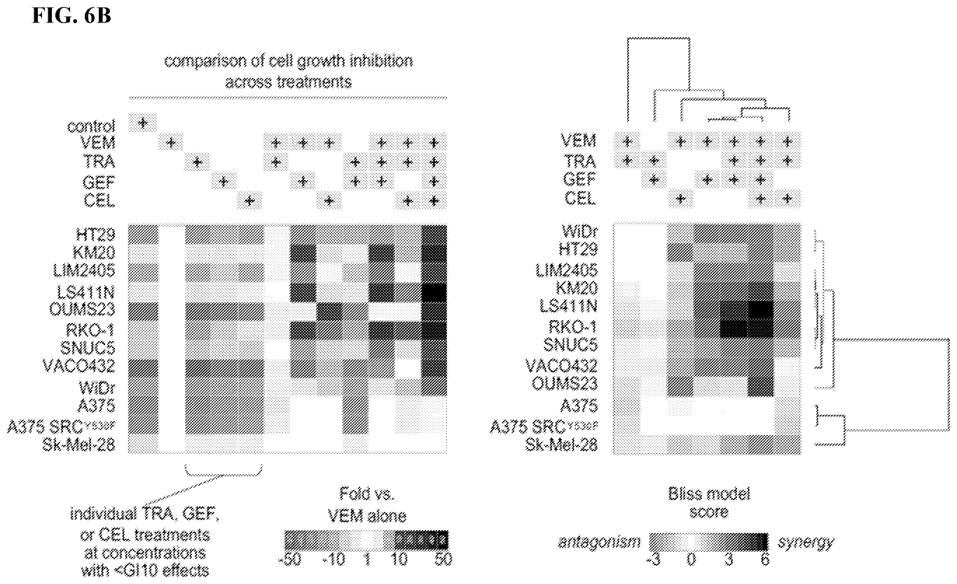

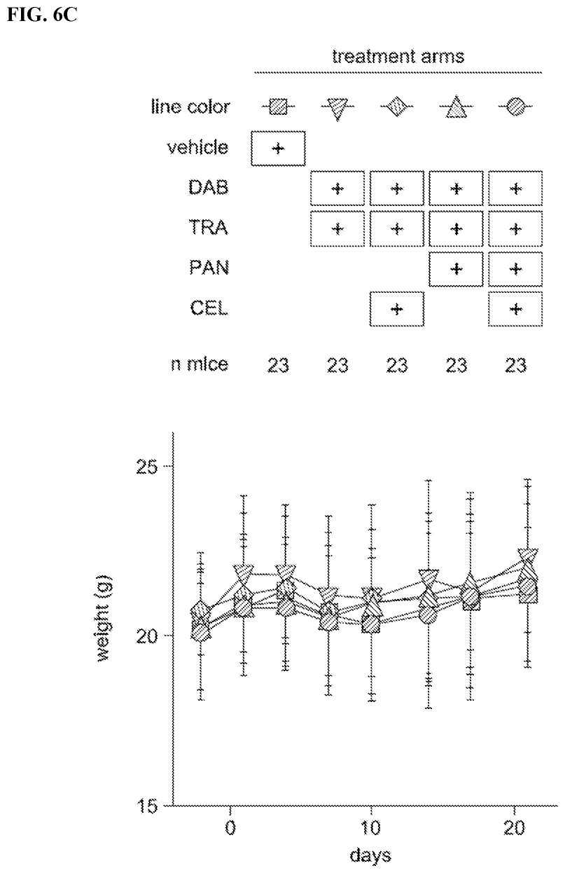

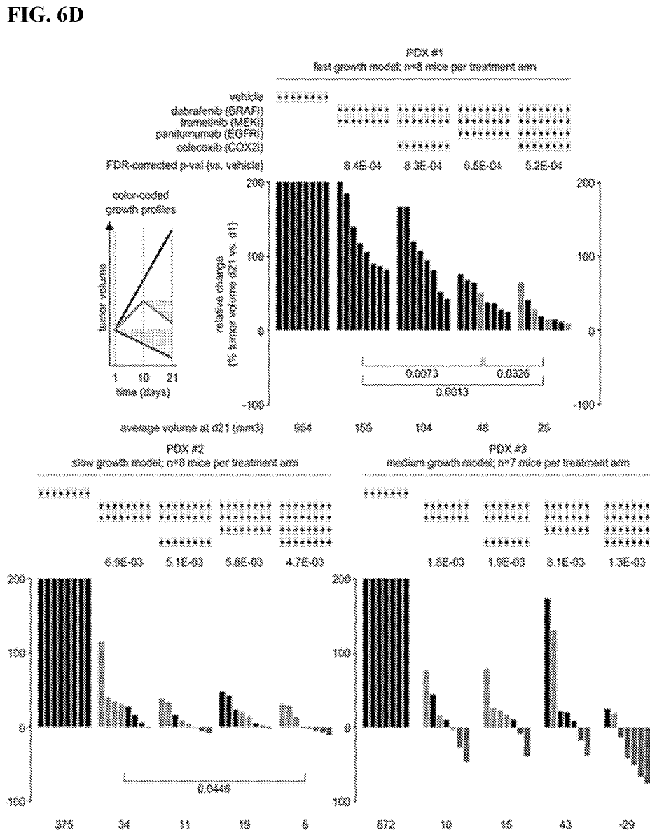

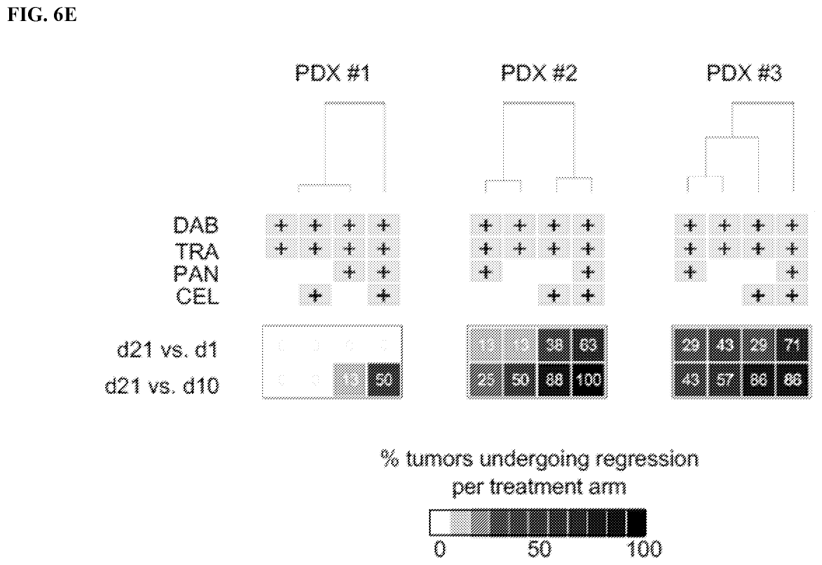

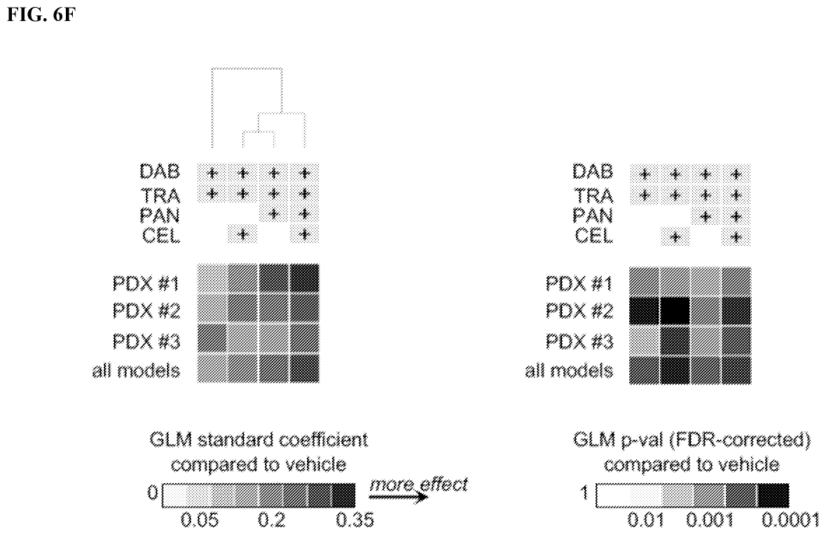

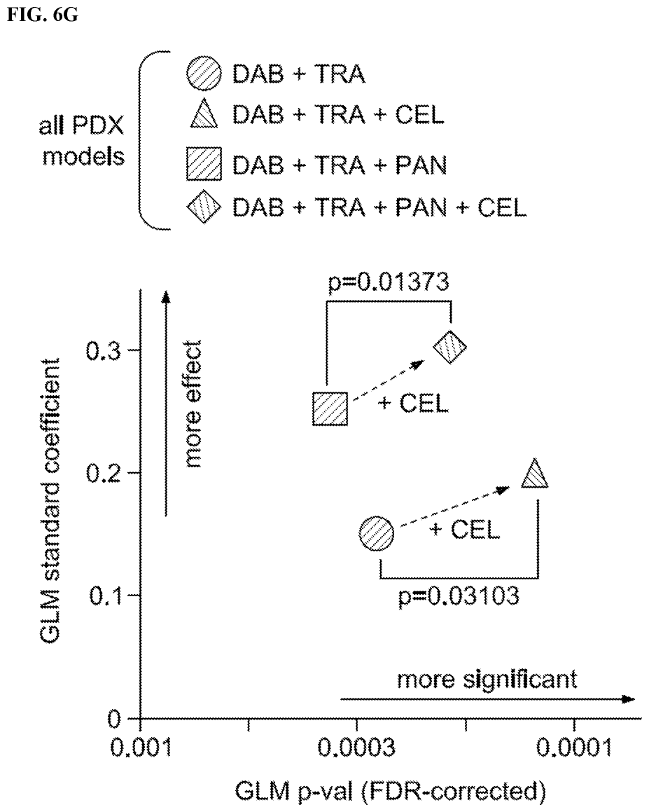

COX2 is a rational drug target, given its robust association with CRC tumor progression in patients²⁴, although no prior clinical trials have focused on BRAF^{V600E} CRC. COX2 inhibitors also represent a practical alternative to SRC-targeting therapies: the COX2 inhibitor, celecoxib, is FDA-approved, has a favorable side-effect profile and is relatively inexpensive. Thus, the logical next step was to test BRAF-targeted therapies in combination with COX2 inhibition. As proof of concept, addition of a COX2 inhibitor produced a consistent, synergistic increase in sensitivity to vemurafenib across a panel of BRAF^{V600E} CRC cell lines, which was not recapitulated in BRAF^{V600E} melanoma (FIG. 6A). Using very low individual drug concentrations of trametinib, gefitinib and celecoxib (GI10 or below), we were able to demonstrate potentiation of cell growth inhibition with combinations of up to four targeted therapies (FIG. 6B, left panel). The addition of celecoxib systematically improved the efficacy of—and synergized with—vemurafenib+trametinib±gefitinib (FIG. 6B, right panel). The most synergistic combination, across BRAF^{V600E} CRC but not melanoma cell lines, was the quadruple treatment arm.

Next, we tested whether addition of celecoxib could improve upon two clinical benchmark regimens: the dabrafenib+trametinib doublet received by the patients from whom the PDX models were derived⁴, and a triplet regimen with addition of the anti-EGFR antibody, panitumumab, which was tested in a subsequent clinical trial⁶. Toxicology studies were conducted prior to efficacy testing: mouse weight, a surrogate for drug toxicity, remained stable over the course of therapy for all inhibitor combinations (FIG. 6C). The critical finding from the efficacy studies was that addition of celecoxib to the clinical trial-tested doublet and triplet drug regimens resulted in consistently superior tumor growth inhibition in all three BRAF^{V600E} CRC PDX models (FIG. 6D). The majority of tumors treated with celecoxib in addition to dabrafenib+trametinib±panitumumab exhibited regression by the second half of the 21-day treatment course (FIGS. 6D-6E). It is notable that 78% of tumors subjected to quadruple therapy with celecoxib exhibited regression, vs. 30% of tumors regressing in the no-celecoxib triplet therapy counterpart arm. Across models, quadruple treatments resulted in the most statistically significant tumor growth inhibition (FIG. 6D; FDR-correct p-values above the bar graphs).

When applying the GLM approach across PDX models, we found that the addition of celecoxib systematically increased effect size; the greatest effect size was observed for the quadruple therapy (FIG. 6F, left panel; FIG. 6G, y-axis). Moreover, addition of celecoxib to any treatment arm resulted in statistically significant improvements in tumor growth inhibition (FIG. 6F, right panel; FIG. 6G, x-axis and arrows).

### Addition of Celecoxib to Current Standard of Care Treatment Results in Durable Tumor Growth Inhibition in BRAF^{V600E} CRC PDXs.

Findings in FIG. 6D prompted us to assess the durability of treatment effects in the two most drug resistant PDXs (PDX #1 and #2 in FIG. 6D). We measured changes in tumor volume for >50 days in mice treated with encorafenib (BRAFi)±panitumumab (EGFRi)±celecoxib (COX2i) (FIGS. 7A, 7B). We found that the triple treatment significantly improved tumor growth inhibition as compared to the dual drug combination, which is a current standard of care⁵ (p-values across measurements underneath the graph in FIGS. 7A-7B). This was confirmed by GLM analysis considering all time points and individual tumor volumes (FIGS. 7D-7E). Increased toxicity was not observed with the addition of celecoxib (FIG. 7E). These results indicate that COX2 inhibition represents a novel, low cost and low toxicity drug-repurposing strategy to overcome therapeutic resistance in BRAF^{V600E} CRC: supplementing encorafenib+panitumumab with celecoxib durably improves tumor growth inhibition.

### DISCUSSION

Despite recent optimization of targeted therapy combinations³⁻⁶, BRAF^{V600E} still predicts the worst prognosis form of mCRC. Thus, we endeavored to uncover orthogonal mediators of compensatory resistance to BRAF, MEK and EGFR targeted inhibitors tested in patients. We discovered that SRC kinases act as a nexus of adaptive, druggable, and EGFR-independent therapeutic resistance in vitro and in vivo. Our findings were reproducible across a variety of inhibitors in the same class, cell lines, and mouse models, and yet were specific to BRAF^{V600E} mCRC. The activation of SRC in response to BRAF±MEK or EGFR therapies did not contribute to MAPK signaling rebound. Instead, we found that SRC activation regulates transcriptional reprogramming via beta-Catenin activation, and is mediated by an upstream pro-inflammatory pathway involving COX2. Addition of celecoxib to inhibitor combinations tested in clinical trials consistently resulted in superior and durable tumor growth suppression in BRAF^{V600E} CRC PDX models, Our study identified unanticipated cooperative dependencies of actionable targets, yielding new strategies to overcome therapeutic resistance (summarized in FIG. 7F).

BRAF^{V600E} is a classic example of how the same activating mutation can play different roles depending on the cancer subtype-specific signaling context. BRAF inhibitors have produced an impressive response rate in BRAF^{V600E} melanoma—but not in CRC³. In CRC, synthetic lethality genetic dropout screens originally found that feedback activation of EGFR promotes intrinsic resistance to BRAF inhibition^{12-14, 25} However, modest responses in patients treated with BRAF+EGFR combination therapy underline why, in situations where predicting therapeutic response cannot be reduced to a single genetic dependency, there is a role for functional proteomic approaches designed to more comprehensively reveal crosstalk between signaling pathways, and to better dissect the dynamic processes induced by drug interventions²⁶⁻²⁹. Here, we used the HT-KAM platform to directly capture the phospho-catalytic fingerprint of kinases in biological extracts, and to identify ranked drug susceptibilities. This elucidated how the concerted rewiring of interdependent signaling pathways drives resistance to BRAF, MEK and EGFR targeted therapies. SRC was identified as a central, conserved mediator of these signaling circuits in BRAF^{V600E} CRC.

SRC kinases are a non-receptor protein tyrosine kinase (NRTK) family of essential pleiotropic mediators of signaling cascades that connect extracellular cues to intracellular programs^{17,18, 30} Knowing that SRC often acts downstream receptor protein tyrosine kinases (RTKs), including in the context of acquired resistance to RAF inhibition⁵⁹⁻⁶⁰, one might expect SRC to be activated by EGFR in response to BRAF-targeted therapy^{12, 13,25} Unexpectedly however, in BRAF^{V600E} CRC, we found that SRC kinases function as integral components of a drug-resistance circuit that is triggered independently of EGFR. Specifically, BRAF^{V600E} CRC cells adapt to targeted therapy by relying on a separate inflammatory loop that funnels through SRC. Possibly even more surprising, our in vivo and in vitro observations (FIGS. 3A-3I) indicate that SRC may have a more dominant role than EGFR in the context of BRAF^{V600E} CRC. In fact, previously discovered transactivation effects of SRC onto EGFR via intra- and extra-cellular mechanisms^{21,31} show that SRC acts as an upstream effector of EGFR. A SRC-driven transactivation mechanism would provide an alternative route to the previously noted feedback-release activation of EGFR via reduced CDC25C phosphatase activity that is initiated by therapeutic inhibition of BRAF/MEK¹³. Together, CDC25C and SRC could functionally complement each other by converging on EGFR to coordinate its activation upon BRAF/MEK-inhibition.

Although SRC kinases are known direct upstream effectors of c-RAF, which regulate the signaling activity of RAF homo-/hetero-dimers³²⁻³⁵, inhibition of SRC did not impact the ERK rebound commonly associated with RAF-therapy resistance. A SRC-driven drug-bypass mechanism might still explain how tumors can efficiently evade RAF-targeting, without acquired resistance mutations. Moreover, SRC kinases can directly promote the activity of other kinases involved in drug resistance, including AKT1³⁶, an essential mediator of EGFR-signaling. BRAF^{V600E} CRC cells may thus compensate for the strain of EGFR-targeting via SRC, releasing cells from their dependency on EGFR to adapt to BRAF+EGFR combination therapies.

In addition to these direct regulatory effects on downstream kinases, SRC kinases are also known to propagate their pro-oncogenic activities via networks of transcription factors³⁷⁻³⁹. In fact, we found that SRC phosphorylates an understudied phospho-site of beta-Catenin (Y654). Upon BRAF±EGFR inhibition, SRC-dependent phosphorylation of Y654 increases beta-Catenin's transcriptional activity, leading to a reprogramming of the transcriptional profiles of BRAF^{V600E} CRC cells. The WNT/beta-catenin signaling pathway is a key driver in the initiation and progression of CRC, differentiating it from other cancers including BRAF-mutated melanoma. Such SRC-driven re-programming mechanism could rapidly and durably re-wire signaling pathways in BRAF^{V600E} CRC cells, effectively promoting adjustment to therapeutic stress.

The signaling plasticity offered by these SRC-dependent mechanisms may usurp and/or reinforce cell-autonomous pathways that drive tumor survival while bypassing other dependencies, including under the influence of BRAF/MEK or EGFR targeted therapies. Altogether, this argues that blocking the pathways that activate SRC kinases represents a logical strategy to reinforce the inhibition of both the RAF/MEK/ERK and EGFR/PI3K/AKT axes, and to prevent the emergence of a therapeutic resilience phenotype (FIG. 7F).

FDA-approved SRC inhibitors are available, however toxicity in combination with other targeted agents is a major concern. This prompted us to determine what upstream pathways activate SRC in order to potentially leverage these mechanisms as clinically actionable targets. Our data show that PGE2-signaling drives SRC activation in BRAF^{V600E} CRC. Despite decades of work on SRC, its contribution as a regulator of cancer-related inflammation has remained largely unexplored. Here, we demonstrated that BRAF^{V600E} CRC cells overcome BRAF±MEK or EGFR therapies by upregulating a pro-survival, auto-/onco-crine, COX2/PGE2/GNAS/SRC/beta-Catenin signaling loop. This adaptive response was not observed in BRAF^{V600E} melanoma cells, highlighting how SRC is embedded in pre-existing signaling networks specific to BRAF^{V600E} CRC. Of note, >90% of CRC tumors also harbor alterations in the WNT signaling pathway, typically an initiating APC mutation⁴⁰. Kinase circuits and drug-response mechanisms are inevitably adapted to the WNT/APC-mutated background of BRAF^{V600E} CRC cells. Both the WNT and G-protein-regulated signaling networks are induced by extracellular inflammatory cues, and both share many intracellular signaling components, such as GSK3b or beta-catenin^{21, 41} This inherent predisposition of BRAF^{V600E} CRC cells to rely on inflammatory pathways to alleviate and withstand drug pressure is underscored by our finding that the SRC-relayed PGE2 signaling cascade causes therapeutic resistance (FIG. 7F).

The production of PGE2 is regulated by the COX2 enzyme. COX2 is a rational target, implicated in intestinal inflammation and, by association, CRC initiation and progression^{24, 42} Although several prior clinical trials testing SRC or COX2 inhibitors in CRC patients have failed to show meaningful clinical activity⁴³⁻⁴⁸, no trial has yet focused on BRAF^{V600E} mCRC, or evaluated these agents in combination with BRAF-targeted therapies. Recent BRAF^{V600E} mCRC clinical trials have proven the feasibility of administering three targeted therapies simultaneously⁵, however, quadruple therapy pushes the limits of acceptability—unless the fourth therapy is inexpensive and has an exceptionally favorable side-effect profile. On both accounts, COX2 is a more attractive drug target than SRC. We showed that adding celecoxib to a current standard of care treatment, encorafenib (BRAFi)+panitumumab (EGFRi)⁵, resulted in sustainable and significant tumor growth inhibition in BRAF^{V600E} CRC PDXs. Addition of the inexpensive, FDA-approved COX2 inhibitor, celecoxib, to BRAF-targeted therapies could be rapidly translated in patients with BRAF^{V600E} mCRC, with few anticipated side-effects.

We attempted to determine retrospectively whether use of celecoxib or other nonsteroidal anti-inflammatory drugs as concomitant medications conferred benefit to CRC patients who participated on clinical trials testing BRAF inhibitor-based therapies, however incomplete data collection and heterogeneous dosing precluded this analysis. Additionally, while the PDX models faithfully recapitulate patients' initial responses to targeted therapies⁶, it is unknown whether or not they can fully predict response, especially as the mice lack a functional immune system. Furthermore, we acknowledge that there may be alternative routes to adaptive resistance, not represented by our models; in which case, it may be possible to leverage the HT-KAM platform to develop predictive biomarkers to tailor therapy most effectively.

In conclusion, our results demonstrate that SRC plays a dominant role in mediating the unresponsiveness of BRAF^{V600E} colorectal tumors to BRAF inhibitors, and suggest that SRC and EGFR act in conserved, complementary, parallel circuits that drive resistance, and can be jointly targeted to restore therapeutic sensitivity. SRC activation is non-redundant with the MAPK pathway. We determined that a COX2-inflammatory pathway drives SRC activation; the effects of inhibiting SRC were recapitulated in vitro and in vivo by targeting COX2. These results argue that drug resistance can result from a combination of pathways that are upregulated, working in concert, and interdependent on each other, such that impeding their coordinated signaling activities is necessary to overcome resistance. The HT-KAM approach can identify a finite number of key cooperative dependencies, offering a curated selection of convergent targets to evaluate. Our hope is that by expanding the scope of investigation, BRAF^{V600E} mCRC will one day become like HER2-positive breast cancer, where what was once a poor prognosis subtype with few treatment choices has been transformed into an opportunity to receive effective targeted therapy options.

### Example 2: Methods

### Cells Lines, Cell Culture Conditions, Genetic Alterations.

Cell lines used in this study were purchased from ATCC or provided by Dr. R.

Bernards; (i) BRAF^{V600E} CRC cells: WiDr, SNUC5, HT29, Colo-205, RKO-1, LIM2405, KM20, LS411N, VACO432, SW1417; CRC MAP3K8^{amp}: OUMS23; (ii) KRAS^{mut} CRC cells: HCT116, LoVo; (iii) BRAF^{V600E} melanoma cells: A375, A375 SRC^{Y530F}, A375 (myr-AKT1), Sk-Mel-28, Mel888. Cells were cultured following ATCC's instructions or as previously described (13).

### Transfections.

siRNA transfections were performed using Lipofectamine 2000 according to the manufacturer's instructions (Invitrogen). Cells were processed 72 hours after siRNA transfection. Control siRNAs and siRNAs targeting CSK were obtained from Dharmacon (siCON: D-001206-13-5 and D-001206-14-20; siCSK: M-003110-02).

### Generation of Knocked Down SRC and Control Stable Cell Lines.

For stable knockdown of SRC, the oligonucleotides containing the shRNA hairpin were annealed and then ligated into the pLKO.1 vector. The two shRNA sequences used against SRC were shSRC #1 (hairpin sequence #TRCN0000199313: 5′-GCTGACAGTTTGTGGCATCTT-3′) (SEQ ID NO:1) and shSRC #2 (hairpin sequence #TRCN0000195339: 5′-CATCCTCAGGAACCAACAATT-3′) (SEQ ID NO:2). Lentiviruses were produced by the UCSF Viracore. WiDr, KM20, LIM2405 and SNUC5 cell lines stably expressing shRNAs control and against SRC (shCON, shSRC #1, shSRC #2) were produced by transduction with the corresponding lentiviruses in the presence of 8 μg/ml polybrene (Sigma-Aldrich). After incubation in growth medium for 72 hours, cells were treated with 2 μg/ml puromycin, the selection antibiotic expressed by the viral vector, to remove non-expressing cells. Puromycin-resistant cell populations were used for the various experiments described in the relevant sections.

### Generation of GNAS Lenti-CRISPR Knockout Cell Lines.

The GNAS lenti-CRISPR knockout cancer cell lines were generated by targeting exon 1 of the human GNAS locus with the CRISPR/Cas9 system as previously described (49, 50). The forward and reverse sgRNA-targeting sequences used were 5′-CACCGCTACAACATGGTCATCCGGG-3′ (SEQ ID NO:3) and 5′-AAACCCCGGATGACCATGTTGTAGC-3′ (SEQ ID NO:4), respectively. Guide sequences were provided by Aska Inoue (Tohoku University). Oligonucleotides for the sgRNAs were phosphorylated and annealed for insertion into BsmBI digested lenti-CRISPR v2 backbone (Addgene cat #52961). Lentiviruses were produced by transfecting HEK293T17 cells with enveloping, packaging, and guide DNAs at a 1:2:3 ratio. Media was collected at 48 and 72 hours post-transfection. Viral particles were concentrated by ultracentrifugation at 28,000 rpm for 4 hours at 4° C. Cancer cell lines were seeded on poly-lysine coated 6-well plates and transduced once the cells reached 70% confluence. Media was refreshed after 48 h and cells were transduced again for 48 h. Polybrene (10 μg/ml) was used to enhance the transduction efficiency. Cells were selected with 1 μg/ml of puromycin for 5 days.

### Generation of Tet-GNAS Active Mutant Cell Lines.

The GNAS active mutant was previously generated by site-directed mutagenesis of arginine 201 to cysteine (51). GNAS^{R201C} cDNA was cloned into the pENTR backbone (pDONR221, ThermoFisher Scientific, Catalog #12536017) using the Gateway cloning BP reaction according to manufacturer protocols (Invitrogen, Catalog #11789020). Activity of the GNAS^{R201C} active mutant was confirmed by cAMP responsible element (CRE) luciferase assay (Dual-Glo Luciferase Assay System, Promega Catalog #E2920) or cAMP immunoassay (R&D Systems, Cat. #KGE002B). The GNAS^{R201C}-pENTR vector was then recombined with the lentiviral vector pLVX-TetOne FLAG Puro (kindly provided by Dr. Krogan's group, UCSF) using the Gateway LR reaction according to the manufacturer protocol (Invitrogen, Cat. #11791020). Viral particles were harvested from HEK293T17 cells and concentrated by ultracentrifugation. Cells were transduced two times for 48 hours and then selected with 1 g/ml puromycin for 5 days.

### Cell Extracts.

For samples to be analyzed with the HT-KAM platform, cells at ˜85% confluency were washed three times with cold PBS and lysed with freshly prepared 1× cell lysis buffer (1 ml per 2.5×10⁶ cells) (lOX Cell Lysis Buffer, Cell Signaling; cat. #9803) complemented with 1× of Halt Protease & Phosphatase (100×, ThermoScientific; cat. #1861281). Cell lysates were collected and spun down at 14,000 rpm for 15 min at 4° C. and supernatants stored at −80° C. For samples to be analyzed by western blot, cell lysates were prepared with RIPA lysis buffer (150 mM NaCl, 0.1% SDS, 1% Nonidet P40, 1% sodium deoxycholate and 10 mM sodium phosphate, pH 7.2) supplemented with protease and phosphatase inhibitors. After clearing by centrifugation at 12,000 rpm for 10 minutes at 4° C., the lysates were analyzed as described in the respective experiments.

### Kinase Inhibitors and Cell Treatment Conditions.

Inhibitors purchased from Selleck Chemicals are: Bosutinib (SKI-606, cat #51014), Celecoxib (SC 58635, cat #S1261), Cetuximab (cat #A2000), Dabrafenib (GSK2118436, cat #S2807), Dasatinib (BMS-354825, cat #S1021), Gefitinib (ZD-1839, cat #S1025), PLX-4720 (cat #S1152), Saracatinib (AZD0530, cat #S1006), Trametinib (GSK1120212, cat #S2673), Valdecoxib (cat #S4049), Vemurafenib (PLX4032, cat #S1267). Panitumumab was provided by Amgen Oncology (Thousand Oaks, CA). Prostaglandin E₂ (PGE2) was purchased from R&D Systems (cat #2296). Drug treatment conditions (concentration and time) related to western blots, ELISA, kinase activity profiles and qRT-PCR, are provided in Table 1.

### Kinase Activity Mapping Assay.

This high throughput kinase-activity mapping (HT-KAM) platform uses arrays of peptides that act as sensors of phosphorylation activity (7). The phospho-catalytic signature of samples is established from simultaneously occurring ATP-consumption tests measured in the presence of individual peptides that are experimentally isolated from each other. Assays are run in 384 well-plates, where each experimental well contains one peptide. The final 8 μL reaction mixtures per well contain: (i) kinase assay buffer (1×KaB: 2.5 mM Tris-HCl (pH7.5), 1 mM MgCl₂, 0.01 mM Na₃VO₄, 0.5 mM □-glycerophosphate, 0.2 mM dithiothreitol (DTT), prepared daily; 10×KaB Cell Signaling cat. #9802), (ii) 250 nM ATP (prepared daily with 1×KaB; Cell Signaling cat. #9804), (iii) 200 μg/ml 11-mer peptide (lyophilized stocks originally prepared as lmg/ml in 1×KaB, 5% DMSO), and (iv) samples made from cell at ˜10 μg/ml total protein extract. Samples are kept on ice and diluted in 1×KaB<30 min before being used. Controls with no-ATP, or no-peptide, or no-sample as well as ATP standards are run side-by-side within each 384-well plate. High-throughput liquid dispensing of all reagents is performed using a Biomek® FX Laboratory Automation Workstation from Beckman Coulter. All reagents are kept on ice and plates on cold blocks until enzymatic reactions are started. Once the dispensing of the reaction mixtures is complete, the plates are incubated for 1 h at 30° C. ATP is detected using Kinase-Glo revealing reagent (Promega; cat. #V3772), which stops the activity of the kinases and produces a luminescent signal that directly correlates with the amount of remaining ATP in the samples. Luminescence is acquired using the Synergy 2 Multi-Mode Microplate Reader from BioTek. Luminescence data are inversely correlated with the amount of kinase activity. For a more detailed description of the peptide sensors design, sequence and connectivity between peptides and kinases, as well as data normalization steps and analysis, refer to: (7, 9, 10, 52, 53). The activity of kinase enzymes is derived from their respective subset of biological peptide targets included in the assay.

### Antibodies and Western Blotting.

For western-blot, samples were denatured by boiling in 1× Laemmli buffer and run on an 8% SDS-PAGE gel. After transfer onto a PVDF membrane and blocking with 3% BSA, the membranes were incubated with primary antibodies overnight at 4° C., washed 3 times with TBST (Tris-buffered saline containing 0.05% Tween-20), incubated with secondary antibodies for 1 hour at room temperature and developed using chemoluminescence (cat #32209 from Pierce).

Antibodies anti-HSP90 was procured from Santa Cruz Biotechnologies (H-114, cat #7947). Anti-phospho Src (Y419) (cat #AF2685) used for IHC was obtained from R&D Systems. Anti-phospho Src Family (Y416) (D49G4, cat #6943) used for western-blots, anti-non-phospho-Src (Y527) (cat #2107); anti-Src (32G6, cat #2123) used for western-blots and anti-Src (cat #2109) used for IHC, anti-phospho-p44/42 MAPK (ERK1/2 T202/204, cat #9101); anti-p44/42 MAPK (ERK1/2, cat #9102), anti-phospho-MAP2K1/2 (MEK1/2 T202/204, cat #9121); anti-MAP2K1/2 (MEK1/2, cat #9122) and anti-CSK (C74C1, cat #4980) were from Cell Signaling; anti-Gsalpha-Subunit (GNAS, cat #371732) was obtained from CalBiochem (now Millipore/Sigma), anti-COX2 was purchased from Spring Bioscience (cat #M3210), Gs alpha subunit, C-terminal (385-394) antibody was purchased from Calbiotech #371732). Secondary antibodies Horseradish peroxidase (HRP)-conjugated were from GE Healthcare (rabbit cat #LNA934).

In an inactive form, SRC is phosphorylated at tyrosine Y530 (Tyr530 in mammalian c-Src; Tyr527 in chicken c-Src) near the C-terminus of SRC. Phosphorylation at Y419 (Tyr416 in chicken c-Src) is associated with SRC activation. The SFK family encompasses 11 members in humans by the Manning classification (Manning et al., 2002) and the numbering for those two key tyrosines varies for some of the other family members. SRC antibodies used in western-blots cross-react to certain extent with other members of the SFK (data not shown).

### ELISA Assay.

To detect secreted PGE2, conditioned media of BRAF^{V600E} CRC cell lines treated with vemurafenib (VEM)±gefitinib (GEF) was collected. Particulates were removed by centrifugation and samples were processed according to the manufacturer protocol (R&D, Catalog KGE004B).

RNA Extraction, cDNA Synthesis and Quantitative Real-Time PCR (qRT-PCR).

RNA was purified using TRIzol followed by DNAse RNase-free digestion (Qiagen). cDNA synthesis was performed using the High Capacity cDNA Reverse Transcription kit (ABI). Primer efficiencies were assessed by serial dilutions. qRT-PCR reactions were performed in QuantStudio™ 5 Real-Time PCR System using SYBR-Green Power master mix (ABI) with default cycling conditions; results were analyzed with the QuantStudio™ 5 analysis software. All mRNA levels were assayed in quadruplicates; dissociation curves were checked and products were run in agarose gels to confirm amplification of only one product. Relative mRNA levels of beta-catenin target genes (MYC, AXIN2, ASCL2, S100A6, LEF1, NOTCH2, SP5) were calculated by the 2\hat{ }^{(ΔΔCt)} method using ACTB and UBC as controls. The sequences (5′→3′) of primers we used to measure the mRNA levels of beta-catenin target and housekeeping genes are the following: NOTCH2 Fwd: CAACCGCAATGGAGGCTATG (SEQ ID NO:5), NOTCH2 Rev: GCGAAGGCACAATCATCAATGTT (SEQ ID NO:6), MYC Fwd: GTCAAGAGGCGAACACACAAC (SEQ ID NO:7), MYC Rev: TTGGACGGACAGGATGTATGC (SEQ ID NO:8), ASCL2 Fwd: AACTTGAGCTGCTGGAGGGACA (SEQ ID NO:9), ASCL2 Rev: TCTTGGCCAGCATGGAAAACTC (SEQ ID NO:10), S100A6 Fwd: GGGAGGGTGACAAGCACAC (SEQ ID NO:11), S100A6 Rev: AGCTTCGAGCCAATGGTGAG (SEQ ID NO: 12), SP5 Fwd: AATGCTGCTGAACTGAATAGA (SEQ ID NO: 13), SP5 Rev: AACCGGTCCTAGCGAAAACC (SEQ ID NO: 14), AXIN2 Fwd: CAACACCAGGCGGAACGAA (SEQ ID NO:15), AXIN2 Rev: GCCCAATAAGGAGTGTAAGGACT (SEQ ID NO:16), LEF1 Fwd: AGAACACCCCGATGACGGA (SEQ ID NO:17), LEF1 Rev: GGCATCATTATGTACCCGGAAT (SEQ ID NO:18), ACTB Fwd: CATGTACGTTGCTATCCAGGC (SEQ ID NO:19), ACTB Rev: CTCCTTAATGTCACGCACGAT (SEQ ID NO:20), UBC Fwd: ATTTGGGTCGCGGTTCTTG (SEQ ID NO:21), UBC Rev: TGCCTTGACATTCTCGATGGT (SEQ ID NO:22). For statistical analysis of qRT-PCR results, we used a t-test, two-sample equal variance (with a two-tailed distribution), to determine the significance of differences in gene expression.

### Cell Viability Assays.

To assess the growth/survival response of cell lines to single or combinatorial drug treatments, we used CellTiter-Glo cell viability assay (Promega; cat #G7571). Cell culture and luminescence readouts were performed in 96- and 384-well plates after 3-day treatments. GI50 corresponds to the concentration of a given drug that causes 50% inhibition of cell growth (GI50). The effects of drug combinations on cell growth were assessed by calculating fold change in VEM-sensitivity and combination index (C.I.) that were experimentally measured from ≥9 individual datapoints around drugs' GI50, i.e. at GI50, 0.5×GI50, 2×GI50 concentrations of each drug per drug combination experiment. To address the particular effects of some drug treatments on some cell lines, the choice of experimental datapoints centered around drugs' GI50 effects was adjusted by including individual datapoints ranging from ≥GI25 to ≤GI75, leading to calculate fold change in VEM-sensitivity and combination index (C.I.) from 12 to 20 individual datapoints, as previously explained (7).

To calculate CI values in FIGS. 2A, 2B, 2E, FIGS. 5C, 5E, 5G and FIG. 6A, we applied the Bliss Independence model (54,55), which uses experimental profiles and avoids inaccuracies that commonly occur with dose-effect curve estimation approaches. CI is calculated as CI=−log 2 (Eab/(Ea*Eb)), where Ea and Eb correspond to the effects of drugs a and b alone at a given concentration, and Eab corresponds to the combined effects of drugs a and b at these same concentrations. In this model, CI>0 indicates synergistic effect, CI=0 indicates additivity effect, CI<0 indicates antagonistic effect.

To compare the effects of triple versus dual drug combinations in FIG. 3B, we calculated CI as follow: CI=−log 2 (Eabc/(Ea*Eb)), where Ea and Eb correspond to the effects of drugs a/VEM, b/GEF alone at a given concentration, and Eabc corresponds to the combined effects of drugs a and b at these same concentrations combined with a third drug (c/DAS) at the cell line-specific GI50 of DAS. The same method was applied to analyze data shown in FIG. 6E.

### Colony Formation Assays.

Colony formation assays were performed as previously described (13). In brief, to test the responses of CRC cells to different treatments, cells were plated in medium containing 10% FBS 24 h prior to being washed with serum-free medium, and cultured for 24 h in medium containing 0.1% serum. After low serum incubation, cells were treated with drugs for 30 min and stimulated by 10% FBS.

### Automated Immunohistochemistry Procedure.

To identify changes in protein expression in tumors from PDXs treated or not with dabrafenib and/or trametinib (FIG. 1E, 4h, FIG. 8B), and tumors from patients (FIGS. 8D, 11A), immunohistochemistry staining was preformed using the Ventana DISCOVERY ULTRA autostainer system hosted at the UCSF Histology and Biomarker Core. A critical advantage of the fully automated DISCOVERY ULTRA pipeline is that, once an IHC protocol is set, all parameters and workflow are automated and repeated identically, thus allowing for all biospecimens to be processed in the exact same way, which further allows for automated image processing and comparative analysis.

Ventana reagents where used, except where noted, according to manufacturer's instructions. Specific settings for each antibody were programed on the DISCOVERY ULTRA, as detailed below. Tissues known to express the marker of interest were included as positive control with each staining run, and during optimization the same tissue was used following the optimized protocol with primary antibody omitted to generate a background reference negative control slide.

Briefly, slides where sectioned at 4 μm thickness, mounted on positively charged slides, and air dried. To increase tissue adhesion slides were baked in oven at 60° C. for a minimum of one hour to a maximum of 24 h. De-paraffinization was done on the DISCOVERY ULTRA in three cycles of 8 min each in EZ Prep solution warmed to 72° C. Antigen retrieval was performed at high temperature, between 95° C. and 100° C., in Cell Conditioning 1 Solution (similar to EDTA) for 4 to 92 min as required by the tissue and antibody combination. Before primary antibody application, inhibitor (specifically Inhibitor CM from the DAB kit) was applied and incubated for between 8 and 20 min. If additional blocking was required to reduce background this was followed by DISCOVERY Goat Ig Block RUO product number 760-600 for 4 to 16 min. Primary antibody was diluted in Discovery Ab Diluent. Species-specific secondary antibody, either OmniMap or HQ and enzyme conjugate, was applied and incubated with low heat between 36 and 37° C. DAB from the DISCOVERY ChromoMap DAB RUO kit was selected as the chromogenic detection for which the Discovery Ultra hard codes the incubation settings. Hematoxylin nuclear counterstain (Cat #760-2021) was applied for 4 min followed by Bluing reagent for additional 4 min. Slides were washed and dehydrated according to Ventana standard operating procedure and coverslipped using 0.17 mm thick glass coverslips and Cytoseal XYL mounting media Richard-Allan Scientific, Cat. #22050262.

To detect phospho-Y419 SRC, phospho-Src (Y419) EGFR rabbit polyclonal supplied by R&D Systems (Cat #AF2685) was used at a titration of 1:50. Cell conditioning in CC1 was performed at 95° C. for 64 min. Inhibitor was applied for 16 min. The primary antibody was incubated at 37° C. for 32 min. Anti-Rabbit HQ was used as the secondary antibody and incubated at 37° C. for 16 min followed by the anti-HQ-HRP enzyme conjugate which was applied for 16 min. DAB was used as the chromogenic detection with hematoxylin selected for the counterstain (as detailed above).

To detect total SRC, TOTAL SRC (36D1) rabbit monoclonal manufactured by Cell Signaling Technology (Cat #2109) was used at a titration of 1:800. Cell conditioning in CC1 was performed at 95° C. for 32 min. Inhibitor was applied for 16 minutes. The primary antibody was incubated at 37° C. for 32 min. OmniMAP anti-rabbit was used as the secondary antibody and incubated at 37° C. for 12 min. DAB was used as the chromogenic detection with hematoxylin selected for the counterstain (as detailed above).

To detect COX2, COX2 (SP21) rabbit monoclonal antibody manufactured by Abcam (Cat #ab16708) was used at a titration of 1:100. Cell conditioning in CC1 was performed at 95° C. for 64 min. Inhibitor was applied for 16 min. The primary antibody was incubated at 37° C. for 32 min. OmniMAP anti-rabbit was used as the secondary antibody and incubated at 37° C. for 8 min. DAB was used as the chromogenic detection with hematoxylin selected for the counterstain (as detailed above).

### Automated Processing and Analysis of IHC Images.

Immunohistochemistry images were processed with the inForm Tissue Finder software (AKOYA Biosciences). The feature recognition algorithms available in inForm, automate the detection and segmentation of tissues and cells, as well as the quantification of immuno-staining intensities (FIGS. 1F, 5I, FIG. 8C). Automation provides consistent, reproducible results and enables comparative studies across specimens/samples/images.

The image processing workflow is a machine-learning process defined by, and adjusted through, an iterative sequence of user-trained modules and variables. The spectral components of each image are first unmixed using a spectral library. Next, all cells composing the immuno-stained tissue image are detected and annotated using tissue- and cell-segmentation modules. These steps locate individual cellular objects by identifying the nuclei from the spectrum corresponding to the hematoxylin stain. Based on nucleus boundaries and definable cytoplasmic/cell membrane features, the inForm software finds and draws the boundaries of each cell. This enables systematic quantification of immuno-staining intensity for each individual cell composing the tissue and across the different tissue types (e.g. tumor vs. stroma). The pattern-recognition detection/segmentation and the immuno-staining intensity scoring of tissues/cells is initially run on 3-to-5 images, and then reiterated using an additional set of 15-to-25 randomly chosen images to further finetune all parameters and validate consistency of processing across images and visualization output. Once the ‘training’ of the inForm algorithm is considered final, batch processing of all images is applied, allowing for systematic processing with identical parameters across 100's of images, and generating results fully comparable between all individual cells from all tissues and biospecimens.

To quantify protein expression at the single cell-level in tumor areas, we automated the scoring of immuno-staining intensities using a binning tool available in the inForm software. The same binning thresholds were used for each antibody (e.g. same 4-bin intensity levels across treatments and across PDXs for SRC (total) to provide comparable results between conditions and tumor cases). However, the thresholds were specifically adapted to each antibody (i.e. different 4-bin intensity levels for SRC vs. SRC pY419 vs. COX2).

It may be noted that the IHC-intensity binning process tends to underestimate both the lowest (0) and highest (+3) intensities per cell. As well, the tissue segmentation tends to miss tissue areas where cells displaying very low immuno-staining intensity (which is inherent to the rules of the algorithms used to train the software to automatically differentiate between tissue types; e.g. tumor, stroma, empty).

### Patient-Derived Xenograft (PDX) Models and BRAF^{V600E} CRC Cell Line Xenograft Studies.

PDX models were established from consenting UCSF patients' tumor biopsy samples as previously described (4, 11). All patients went on to receive dabrafenib+trametinib as part of a clinical trial (4). JAX NOD scid gamma mice bearing subcutaneous PDXs were randomized into vehicle or treatment groups when tumor volumes reached 100-150 mm³ with rolling enrollment. The mice (7 to 8 per group) were treated with vehicle (0.1% Tween-20 or 0.5% hydroxypropyl methylcellulose and 0.2% Tween-80) or targeted therapies for 21 days in the University of California San Francisco Preclinical Therapeutics Core (San Francisco, CA). Inhibitors administered by oral gavage (PO) were purchased from Selleck Chemicals (Houston, TX) and dosed daily (QD) as follows: celecoxib 50 mg/kg PO, QD; dabrafenib 30 mg/kg PO, QD; dasatinib 20 mg/kg PO, QD; gefitinib 50 mg/kg PO, QD; saracatinib 25 mg/kg PO, QD; trametinib 0.6 mg/kg PO, QD; vemurafenib 50 mg/kg PO, QD. For WiDr and KM20 cell line xenograft studies, respectively 8 and 7 mice per treatment group were dosed daily (QD) with a combination of the following inhibitors: PLX4702 (50 mg/kg/day orally), Gefitinib (50 mg/kg/day orally), Dasatinib (50 mg/kg/day orally), Saracatinib (25 mg/kg/day orally). Panitumumab was provided by Amgen Oncology (Thousand Oaks, CA) and was used at 200 μg by intraperitoneal injection twice weekly.

Mice were monitored for signs of toxicity (e.g. weight loss) and tumor size was evaluated twice per week by digital caliper measurements. The 15% body weight reduction threshold for holding drug was not met. The same procedures were followed for cell-line derived xenograft models. All protocols were approved by the Institutional Animal Care and Use Committee (IACUC).

To test the significance of the changes in tumor volume over time between treatment arms and vehicle in FIGS. 3D-3I, FIGS. 6D, 6F, 6G, FIGS. 7A, 7B, we use the following statistical tests: Student t-test, generalized Linear Model (GLM), GLM p-values corrected for false discovery rate (FDR). Besides plotting the relative tumor volume in FIGS. 3D, 3E, FIG. 6D, FIGS. 7A, 7B, we also calculated effect size measured as the GLM standard coefficient. GLM was applied to each tumor model separately, or combined. In FIG. 7B, none of the mice treated with ENC alone were available at the last two time points (euthanized due to tumor size), which is why no p-value could be calculated and is represented as an ‘x’ in the table underneath the plot.

### Patient Specimens.

Tumor specimens were used in research following patient consent and approval by the UCSF Institutional Review Board (#12-09139 and 13-2574).

### Statistics and Reproducibility.

We provide general information on how statistical analyses of data were conducted, and general information on reproducibility of experiments.

For the analysis leading to the heatmap in FIG. 1A, the average value of ATP consumption in sample-containing wells measured across 228 peptides and 14 peptide-free controls was used for internal normalization for each experimental run (i.e. mean-centering value established from 242 datapoints/wells per 384-well plate; previous explained in references (7, 9, 10)). Other normalization schemes were used for further analysis and cross-validation (i.e. (i) subset of 14 peptide-free control wells (i.e. cell extract alone), or (ii) subset of 16 Y/S/T-free peptides, or (iii) subset of 63 reference peptides). The activity per-peptide was then calculated as the difference in ATP consumption between each peptide and the internal mean. ATP consumption measurements associated with each peptide were then averaged across all biological and technical replicates. Next, phosphorylation activity profiles across all individual 228 peptides were compared between treated (VEM or VEM+GEF or VEM+CET) and control (UNT) (calculated as the difference in ATP-consumption). Finally, phospho-catalytic activity signatures measured across the 228-peptide sensors were subjected to unsupervised hierarchical clustering. Phospho-catalytic activities are coded based on the relative level of activity measured in presence of each peptide for each treatment, from for ATP consumption lower than then one measured in untreated cells, to ATP consumption for equal to the one measured in untreated cells (white), to ATP consumption higher than then one measured in untreated cells.

For the analysis to generate heatmaps in FIG. 1B (and related plots in FIGS. 1C, 1D), the activity of kinases was calculated as the average of the phosphorylation activities measured in presence of their respective biological peptide subsets (i.e., derived from values/calculations used to generate FIG. 1A). Here, we systematically converted peptide-phosphorylation profiles into kinase activity signatures for individual kinases and kinase families that were detected with >3 distinct biological peptide sensors and available in the peptide library.

In FIGS. 3D, 3E, FIG. 7C, 7D, changes in tumor volume at each time point are shown as the mean volume of all tumors per treatment arm (+/−standard error (SE)). Student t-test comparing treatment versus vehicle, or between drug treatment arms, are shown.

In FIG. 6D, changes in tumor volume converted to cumulative tumor volume, are shown for all individual tumors per treatment arm at day-21 (final time point to assess and compare the efficacy of different drug combinations). Significance comparing treatment versus vehicle at day-21 were calculated, and p-values (Student t-test) are shown in the plot.

In FIGS. 3F-3I, FIGS. 6F, 6G, FIGS. 7C, 7D, we used a generalized linear model (GLM) to analyze profiles of tumor growth. We first zero-normalized the data compared to control vehicle per model and per time point. We then ran a GLM (available in R) on the normalized data to test association of change in tumor volume between treatment and vehicle across all time points. The effect size of each treatment compared to vehicle was calculated (i.e., GLM standard coefficient) along with significance (i.e., False Discovery Rate (FDR)-corrected GLM p-values). This GLM approach and data normalization also allowed us to compare the effects of different treatments (e.g. with or without dasatinib (FIGS. 3H, 3I); with or without celecoxib (FIG. 6G, FIG. 7D)), as well as to combine the distinct models (e.g., all cell line tumor xenografts (FIG. 3F, 3H), or all PDXs (FIGS. 3G, 3I, FIGS. 6F, 6G, FIGS. 7C, 7D) to measure the overall effect of drug combinations and their significance.

Other statistical and predictive methods to compare sample groups and reproducibility between signatures include unsupervised or semi-supervised hierarchical clustering using Euclidean distance or (Absolute) Correlation (centered or uncentered) and Ward linkage or complete or average linkage to group phospho-activity signatures based on their similarities or differences; False Discovery Rate (FDR/BH)-corrected or not Student t-test, FDR-corrected or not Wilcoxon rank sum test (p-val<0.05); generalized linear model (GLM)REFERENCES.

### Tables

TABLE 1
GI50 concentrations (uM) of vemurafenib, gefitinib
and dasatinib (i.e., leading to 50% growth inhibition
after 3 days of treatment compared to control)

| cell line name | vemurafenib | gefitinib | dasatinib |
|---|---|---|---|
| HT29 | 1.75 | 25 | 8 |
| KM20 | 2 | 20 | 0.8 |
| LIM2405 | 0.15 | 30 | 1.5 |
| LS411N | 1.45 | 30 | 3 |
| OUMS23 | 1.4 | 40 | 1.9 |
| RKO1 | 1.7 | 40 | 10 |
| SNUC5 | 2.25 | 30 | 6.5 |
| VACO432 | 1.15 | 7.5 | 2 |
| WiDr | 1.5 | 10 | 0.9 |
| A375 | 0.01 | 30 | 3.5 |
| Sk-Mel-28 | 0.15 | 20 | 6.5 |

TABLE 2
Drug treatment conditions per figure panel related to protein detection
assays and gene expression (Western Blot, HT-KAM, ELISA, qRT-PCR)

| | | | Dual | | | | |
|---|---|---|---|---|---|---|---|
| Figure | Cell | Single | treatment | Dual | Treatment | | |
| panel # | line | treatment | #1 | treatment #2 | time | Media | Assay |
| Figure | WiDr | VEM @ GI50 | VEM @ | VEM @ | 8 h | 1% | High |
| 1a-b | | | GI50 + | GI50 + | | FBS | throughput |
| | | | GEF @ | CET @ 1/4 | | | Kinase |
| | | | 1/4 GI50 | GI50 | | | Activity |
| | | | | | | | Mapping |
| | | | | | | | assay (HT- |
| | | | | | | | KAM) |
| Figure | WiDr | VEM @ GI50 | VEM @, | n.a. | 20 h | 1% | western |
| 3a | | | GI50 + | | | FBS | blot |
| | | | GEF @, | | | | |
| | | | 1/4 GI50 | | | | |
| Figure | KM20 | VEM @ GI50 | VEM @ | n.a. | 20 h | 1% | western |
| 3a | | | GI50 + | | | FBS | blot |
| | | | GEF @ | | | | |
| | | | 1/4 GI50 | | | | |
| Figure | LIM24 | VEM @ GI50 | VEM @ | n.a. | 20 h | 1% | western |
| 3a | 05 | | GI50 + | | | FBS | blot |
| | | | GEF @ | | | | |
| | | | 1/4 GI50 | | | | |
| Figure | HT29 | VEM @ GI50 | VEM @ | n.a. | 20 h | 1% | western |
| 3a | | | GI50 + | | | FBS | blot |
| | | | GEF @, | | | | |
| | | | 1/4 GI50 | | | | |
| Figure | WiDr | VEM @ GI50 | VEM @ | VEM @ | 20 h | 1% | western |
| 4a | | | GI50 + | GI50 + | | FBS | blot |
| | | | GEF @ | DAS @ | | | |
| | | | 1/4 GI50 | 1/4 GI50 | | | |
| Figure | KM20 | VEM @ GI50 | VEM @ | VEM @ | 20 h | 1% | western |
| 4a | | | GI50 + | GI50 + | | FBS | blot |
| | | | GEF @ | DAS @ | | | |
| | | | 1/4 GI50 | 1/4 GI50 | | | |
| Figure | LIM24 | VEM @ GI50 | VEM @ | VEM @ | 20 h | 1% | western |
| 4a | 05 | | GI50 + | GI50 + | | FBS | blot |
| | | | GEF @ | DAS @, | | | |
| | | | 1/4 GI50 | 1/4 GI50 | | | |
| Figure | HT29 | VEM @ GI50 | VEM @ | VEM @ 1/2 | 8 h | 5% | qRT-PCR |
| 4b | | | 1/2 GI50 + | GI50 + DAS | | FBS | |
| | | | GEF @ | @ 1/2 GI50 | | | |
| | | | 1/2 GI50 | | | | |
| Figure | KM20 | VEM @ GI50 | VEM @, | VEM @ 1/2 | 8 h | 5% | qRT-PCR |
| 4b | | | 1/2 GI50 + | GI50 + | | FBS | |
| | | | GEF @ | DAS @ | | | |
| | | | 1/2 GI50 | 1/2 GI50 | | | |
| Figure | LIM24 | VEM @ GI50 | VEM @ | VEM @ 1/2 | 8 h | 5% | qRT-PCR |
| 4b | 05 | | 1/2 GI50 + | GI50 + DAS | | FBS | |
| | | | GEF @ | @ 1/2 GI50 | | | |
| | | | 1/2 GI50 | | | | |
| Figure | SNUC | VEM@ GI50 | VEM @ | VEM @ 1/2 | 8 h | 5% | qRT-PCR |
| 4b | 5 | | 1/2 GI50 + | GI50 + DAS | | FBS | |
| | | | GEF @ | @ 1/2 GI50 | | | |
| | | | 1/2 GI50 | | | | |
| Figure | LS411 | VEM @ GI50 | VEM @, | VEM @ 1/2 | 8 h | 5% | qRT-PCR |
| 4b | N | | 1/2 GI50 + | GI50 + DAS | | FBS | |
| | | | GEF @ | @ 1/2 GI50 | | | |
| | | | 1/2 GI50 | | | | |
| Figure | VACO | VEM @ GI50 | VEM @, | VEM @ 1/2 | 8 h | 5% | qRT-PCR |
| 4b | 4232 | | 1/2 GI50 + | GI50 + DAS | | FBS | |
| | | | GEF @ | @ 1/2 GI50 | | | |
| | | | 1/2 GI50 | | | | |
| Figure | RKO1 | VEM @ GI50 | VEM @ | VEM @ 1/2 | 8 h | 5% | qRT-PCR |
| 4b | | | 1/2 GI50 + | GI50 + DAS | | FBS | |
| | | | GEF @ | @ 1/2 GI50 | | | |
| | | | 1/2 GI50 | | | | |
| Figure | WiDr | VEM @ GI50 | VEM @ | VEM @ 1/2 | 8 h | 5% | qRT-PCR |
| 4b | | | 1/2 GI50 + | GI50 + | | FBS | |
| | | | GEF @ | DAS @, | | | |
| | | | 1/2 GI50 | 1/2 GI50 | | | |
| Figure | WiDr | VEM @ GI50 | VEM @ | n.a. | 24 h | 0.25% | ELISA |
| 5a | | | GI50 + | | | FBS | |
| | | | GEF @ | | | | |
| | | | 1/2 GI50 | | | | |
| Figure | KM20 | VEM @ GI50 | VEM @ | n.a. | 24 h | 0.25% | ELISA |
| 5a | | | GI50 + | | | FBS | |
| | | | GEF @ | | | | |
| | | | 1/2 GI50 | | | | |
| Figure | LIM24 | VEM @ GI50 | VEM @ | n.a. | 24 h | 0.25% | ELISA |
| 5a | 05 | | GI50 + | | | FBS | |
| | | | GEF @ | | | | |
| | | | 1/2 GI50 | | | | |
| Figure | SNUC | VEM @ GI50 | VEM @ | n.a. | 24 h | 0.25% | ELISA |
| 5a | 5 | | GI50 + | | | FBS | |
| | | | GEF @ | | | | |
| | | | 1/2 GI50 | | | | |
| Figure | WiDr | PGE2 @ | n.a. | n.a. | 8 h | 0.25% | western |
| 5b | | 50pg/mL/1.E5 | | | | FBS | blot |
| | | cells, twice | | | | | |
| Figure | KM20 | PGE2 @ | n.a. | n.a. | 8 h | 0.25% | western |
| 5b | | 50pg/mL/1.E5 | | | | FBS | blot |
| | | cells, twice | | | | | |
| Figure | LIM24 | PGE2 @ | n.a. | n.a. | 8 h | 0.25% | western |
| 5b | 05 | 50pg/mL/1.E5 | | | | FBS | blot |
| | | cells, twice | | | | | |
| Figure | HT29 | PGE2 @ | n.a. | n.a. | 8 h | 0.25% | western |
| 5b | | 50pg/mL/1.E5 | | | | FBS | blot |
| | | cells, twice | | | | | |
| Figure | WiDr | VEM @ GI50 | n.a. | n.a. | 8 h | 5% | western |
| 5f | GNAS | | | | | FBS | blot |
| (bottom | CRISP | | | | | | |
| panel) | R-KO | | | | | | |
| Figure | KM20 | VEM @ GI50 | n.a. | n.a. | 8 h | 5% | western |
| 5f | GNAS | | | | | FBS | blot |
| (bottom | CRISP | | | | | | |
| panel) | R-KO | | | | | | |
| Figure | SNUC | VEM @ GI50 | n.a. | n.a. | 8 h | 1% | western |
| 5f | 5 | | | | | FBS | blot |
| (bottom | GNAS | | | | | | |
| panel) | CRISP | | | | | | |
| | R-KO | | | | | | |
| Figure | WiDr | VEM @ GI50 | n.a. | n.a. | 8 h | 5% | western |
| 8A | | | | | | FBS | blot |
| Figure | HT29 | VEM @ GI50 | n.a. | n.a. | 7 h | 5% | western |
| 8A | | | | | | FBS | blot |
| Figure | KM20 | VEM @ GI50 | n.a. | n.a. | 8 h | 5% | western |
| 8A | | | | | | FBS | blot |
| Figure | LIM24 | VEM @ GI50 | n.a. | n.a. | 7 h | 1% | western |
| 8A | 05 | | | | | FBS | blot |
| Figure | SNUC | VEM @ GI50 | n.a. | n.a. | 8 h | 1% | western |
| 8A | 5 | | | | | FBS | blot |
| Figure | WiDr | VEM @ GI50 | VEM @ | VEM @, 1/2 | 0 h, 8 h, | 5% | western |
| 11A | | | 1/2 GI50 + | GI50 + | 24 h, 48 h, | FBS | blot |
| | | | GEF @ | DAS @ | 72 h | | |
| | | | 1/2 GI50 | 1/2 GI50 | | | |
| Figure | HT29 | VEM @ GI50 | VEM @ | VEM @, 1/2 | 0 h, 8 h, | 5% | western |
| 11A | | | 1/2 GI50 + | GI50 + DAS | 24 h, 48 h, | FBS | blot |
| | | | GEF @ | @ 1/2 GI50 | 72 h | | |
| | | | 1/2 GI50 | | | | |
| Figure | KM20 | VEM @ GI50 | VEM @ | VEM @, 1/2 | 0 h, 8 h, | 5% | western |
| 11A | | | 1/2 GI50 + | GI50 + DAS | 24 h, 48 h, | FBS | blot |
| | | | GEF @ | @, 1/2 | 72 h | | |
| | | | 1/2 GI50 | GI50 | | | |
| Figure | LIM24 | VEM @ GI50 | VEM @ | VEM @ 1/2 | 0 h, 8 h, | 5% | western |
| 11A | 05 | | 1/2 GI50 + | GI50 + DAS | 24 h, 48 h, | FBS | blot |
| | | | GEF @ | @ 1/2 GI50 | 72 h | | |
| | | | 1/2 GI50 | | | | |
| Figure | SNUC | VEM @ GI50 | VEM @ | VEM @ 1/2 | 0 h, 8 h, | 5% | western |
| 11A | 5 | | 1/2 GI50 + | GI50 + DAS | 24 h, 48 h, | FBS | blot |
| | | | GEF @ | @ 1/2 GI50 | 72 h | | |
| | | | 1/2 GI50 | | | | |
| Figure | LS411 | VEM @ GI50 | VEM @ | VEM @ 1/2 | 0 h, 8 h, | 5% | western |
| 11A | N | | 1/2 GI50 + | GI50 + DAS | 24 h, 48 h, | FBS | blot |
| | | | GEF @ | @ 1/2 GI50 | 72 h | | |
| | | | 1/2 GI50 | | | | |
| Figure | VACO | VEM @ GI50 | VEM @ | VEM @ 1/2 | 0 h, 8 h, | 5% | western |
| 11A | 4232 | | 1/2 GI50 + | GI50 + DAS | 24 h, 48 h, | FBS | blot |
| | | | GEF @ | @ 1/2 | 72 h | | |
| | | | 1/2 GI50 | GI50 | | | |
| Figure | RKO1 | VEM @ GI50 | VEM @ | VEM @ 1/2 | 0 h, 8 h, | 5% | western |
| 11A | | | 1/2 GI50 + | GI50 + DAS | 24 h, 48 h, | FBS | blot |
| | | | GEF @ | @ 1/2 | 72 h | | |
| | | | 1/2 GI50 | GI50 | | | |
| Figure | WiDr | VEM @ GI50 | VEM @ | VEM @ | 20 h | 1% | western |
| 11C | | | GI50 + | GI50 + | | FBS | blot |
| | | | GEF @ | DAS @ | | | |
| | | | 1/4 GI50 | 1/4 GI50 | | | |
| Figure | KM20 | VEM @ GI50 | VEM @ | VEM @ | 20 h | 1% | western |
| 11C | | | GI50 + | GI50 + | | FBS | blot |
| | | | GEF @, | DAS @, | | | |
| | | | 1/4 GI50 | 1/4 GI50 | | | |
| Figure | LIM24 | VEM @ GI50 | VEM @, | VEM @ | 20 h | 1% | western |
| 11C | 05 | | GI50 + | GI50 + | | FBS | blot |
| | | | GEF @ | DAS @ 1/4 | | | |
| | | | 1/4 GI50 | GI50 | | | |
| Figure | SNUC | VEM @ GI50 | VEM @ | VEM @ | 20 h | 1% | western |
| 11C | 5 | | GI50 + | GI50 + | | FBS | blot |
| | | | GEF @ | DAS @, 1/4 | | | |
| | | | 1/4 GI50 | GI50 | | | |
| Figure | WiDr | PGE2 @, | n.a. | n.a. | 8 h | 0.25% | western |
| 12A | | 50pg/mL/1.E5 | | | | FBS | blot |
| | | cells, twice | | | | | |
| Figure | KM20 | PGE2 @ | n.a. | n.a. | 8 h | 0.25% | western |
| 12A | | 50pg/mL/1.E5 | | | | FBS | blot |
| | | cells, twice | | | | | |
| Figure | VACO | PGE2 @ | n.a. | n.a. | 8 h | 0.25% | western |
| 12A | 4232 | 50pg/mL/1.E5 | | | | FBS | blot |
| | | cells, twice | | | | | |
| Figure | WiDr | VEM @ GI50 | 1.a. | n.a | 0 h, 8 h, | 5% | western |
| 12B | iGNAS | | | | 24 h, 48 h | FBS | blot |
| | (R201 | | | | | | |
| | C) | | | | | | |
| Figure | HT29 | VEM @ GI50 | 1.a. | n.a. | 0 h, 8 h, | 5% | western |
| 12B | iGNAS | | | | 24 h, 48 h | FBS | blot |
| | (R201 | | | | | | |
| | C) | | | | | | |
| Figure | LIM24 | VEM @ GI50 | n.a. | n.a. | 0 h, 8 h, | 5% | western |
| 12B | 05 | | | | 24 h, 48 h | FBS | blot |
| | iGNAS | | | | | | |
| | (R201 | | | | | | |
| | C) | | | | | | |
| Figure | WiDr | VEM @ GI50 | n.a. | n.a | 0 h, 8 h, | 5% | western |
| 12C | GNAS | | | | 24 h, 48 h | FBS | blot |
| | CRISP | | | | | | |
| | R-KO | | | | | | |
| Figure | KM20 | VEM @ GI50 | n.a. | n.a. | 0 h, 8 h, | 5% | western |
| 12C | GNAS | | | | 24 h, 48 h | FBS | blot |
| | CRISP | | | | | | |
| | R-KO | | | | | | |
| Figure | SNUC | VEM @ GI50 | n.a. | n.a. | 0 h, 8 h, | 5% | western |
| 12C | 5 | | | | 24 h, 48 h | FBS | blot |
| | GNAS | | | | | | |
| | CRISP | | | | | | |
| | R-KO | | | | | | |

TABLE 3
Primer sequences used to measure mRNA
levels of beta-catenin target geens,
and housekeeping genes

| Gene | Orientation | Sequence (5′ -> 3′) |
|---|---|---|
| NOTCH2 | Forward | CAACCGCAATGGAGGCTATG |
| | | (SEQ ID NO: 5) |
| NOTCH2 | Reverse | GCGAAGGCACAATCATCAATGTT |
| | | (SEQ ID NO: 6) |
| MYC | Forward | GTCAAGAGGCGAACACACAAC |
| | | (SEQ ID NO: 7) |
| MYC | Reverse | TTGGACGGACAGGATGTATGC |
| | | (SEQ ID NO: 8) |
| ASCL2 | Forward | AACTTGAGCTGCTGGAGGGACA |
| | | (SEQ ID NO: 9) |
| ASCL2 | Reverse | TCTTGGCCAGCATGGAAAACTC |
| | | (SEQ ID NO: 10) |
| S100A6 | Forward | GGGAGGGTGACAAGCACAC |
| | | (SEQ ID NO: 11) |
| S100A6 | Reverse | AGCTTCGAGCCAATGGTGAG |
| | | (SEQ ID NO: 12) |
| SP5 | Forward | AATGCTGCTGAACTGAATAGA |
| | | (SEQ ID NO: 13) |
| SP5 | Reverse | AACCGGTCCTAGCGAAAACC |
| | | (SEQ ID NO: 14) |
| AXIN2 | Forward | CAACACCAGGCGGAACGAA |
| | | (SEQ ID NO: 15) |
| AXIN2 | Reverse | GCCCAATAAGGAGTGTAAGGACT |
| | | (SEQ ID NO: 16) |
| LEF1 | Forward | AGAACACCCCGATGACGGA |
| | | (SEQ ID NO: 17) |
| LEF1 | Reverse | GGCATCATTATGTACCCGGAAT |
| | | (SEQ ID NO: 18) |
| ACTB | Forward | CATGTACGTTGCTATCCAGGC |
| | | (SEQ ID NO: 19) |
| ACTB | Reverse | CTCCTTAATGTCACGCACGAT |
| | | (SEQ ID NO: 20) |
| UBC | Forward | ATTTGGGTCGCGGTTCTTG |
| | | (SEQ ID NO: 21) |
| UBC | Reverse | TGCCTTGACATTCTCGATGGT |
| | | (SEQ ID NO: 22) |

### REFERENCES

1. Venderbosch S, Nagtegaal I D, Maughan T S, Smith C G, Cheadle J P, Fisher D, Kaplan R, Quirke P, Seymour M T, Richman S D, Meijer G A, Ylstra B, Heideman D A, de Haan A F, Punt C J, Koopman M. Mismatch repair status and BRAF mutation status in metastatic colorectal cancer patients: a pooled analysis of the CAIRO, CAIRO2, COIN, and FOCUS studies. Clin Cancer Res. 2014; 20(20):5322-30. doi: 10.1158/1078-0432.CCR-14-0332. PubMed PMID: 25139339; PMCID: PMC4201568.
2. Morris V, Overman M J, Jiang Z Q, Garrett C, Agarwal S, Eng C, Kee B, Fogelman D, Dasari A, Wolff R, Maru D, Kopetz S. Progression-free survival remains poor over sequential lines of systemic therapy in patients with BRAF-mutated colorectal cancer. Clin Colorectal Cancer. 2014; 13(3):164-71. doi: 10.1016/j.clcc.2014.06.001. PubMed PMID: 25069797; PMCID: PMC4266576.
3. Kopetz S, Desai J, Chan E, Hecht J R, O'Dwyer P J, Maru D, Morris V, Janku F, Dasari A, Chung W, Issa J P, Gibbs P, James B, Powis G, Nolop K B, Bhattacharya S, Saltz L. Phase II Pilot Study of Vemurafenib in Patients With Metastatic BRAF-Mutated Colorectal Cancer. J Clin Oncol. 2015; 33(34):4032-8. doi: 10.1200/JCO.2015.63.2497. PubMed PMID: 26460303; PMCID: PMC4669589.
4. Corcoran R B, Atreya C E, Falchook G S, Kwak E L, Ryan D P, Bendell J C, Hamid O, Messersmith W A, Daud A, Kurzrock R, Pierobon M, Sun P, Cunningham E, Little S, Orford K, Motwani M, Bai Y, Patel K, Venook A P, Kopetz S. Combined BRAF and MEK Inhibition With Dabrafenib and Trametinib in BRAF V600-Mutant Colorectal Cancer. J Clin Oncol. 2015; 33(34):4023-31. doi: 10.1200/JCO.2015.63.2471. PubMed PMID: 26392102; PMCID: PMC4669588.
5. Kopetz S, Grothey A, Yaeger R, Van Cutsem E, Desai J, Yoshino T, Wasan H, Ciardiello F, Loupakis F, Hong Y S, Steeghs N, Guren T K, Arkenau H T, Garcia-Alfonso P, Pfeiffer P, Orlov S, Lonardi S, Elez E, Kim T W, Schellens J H M, Guo C, Krishnan A, Dekervel J, Morris V, Calvo Ferrandiz A, Tarpgaard L S, Braun M, Gollerkeri A, Keir C, Maharry K, Pickard M, Christy-Bittel J, Anderson L, Sandor V, Tabernero J. Encorafenib, Binimetinib, and Cetuximab in BRAF V600E-Mutated Colorectal Cancer. N Engl J Med. 2019; 381(17):1632-43. doi: 10.1056/NEJMoa1908075. PubMed PMID: 31566309.
6. Corcoran R B, Andre T, Atreya C E, Schellens J H M, Yoshino T, Bendell J C, Hollebecque A, McRee A J, Siena S, Middleton G, Muro K, Gordon M S, Tabernero J, Yaeger R, O'Dwyer P J, Humblet Y, De Vos F, Jung A S, Brase J C, Jaeger S, Bettinger S, Mookerjee B, Rangwala F, Van Cutsem E. Combined BRAF, EGFR, and MEK Inhibition in Patients with BRAF(V600E)-Mutant Colorectal Cancer. Cancer Discov. 2018; 8(4):428-43. doi: 10.1158/2159-8290.CD-17-1226. PubMed PMID: 29431699; PMCID: PMC5882509.
7. Coppe J P, Mori M, Pan B, Yau C, Wolf D M, Ruiz-Saenz A, Brunen D, Prahallad A, Cornelissen-Steijger P, Kemper K, Posch C, Wang C, Dreyer C A, Krijgsman O, Lee P R E, Chen Z, Peeper D S, Moasser M M, Bernards R, van't Veer L J. Mapping phospho-catalytic dependencies of therapy-resistant tumours reveals actionable vulnerabilities. Nat Cell Biol. 2019; 21(6):778-90. doi: 10.1038/s41556-019-0328-z. PubMed PMID: 31160710.
8. Olow A, Chen Z, Niedner R H, Wolf D M, Yau C, Pankov A, Lee E P, Brown-Swigart L, van't Veer L J, Coppe J P. An Atlas of the Human Kinome Reveals the Mutational Landscape Underlying Dysregulated Phosphorylation Cascades in Cancer. Cancer Res. 2016; 76(7):1733-45. doi: 10.1158/0008-5472.CAN-15-2325-T. PubMed PMID: 26921330; PMCID: PMC4907268.
9. Mori M, Pan B, Coppe J-P. Hig-Throughput Kinase Activiy Mapping (HT-KAM) system: biochemical assay. Nature Protocol Exchange. 2019. doi: 10.1038/protex.2019.029.
10. Yau C, Wolf D M, Coppe J. High-Throughput Kinase Activity Mapping (HT-KAM) system: analysis of phospho-catalytic profiles. Nature Protocol Exchange. 2019. doi: 0.1038/protex.2019.030.
11. Rajaram S, Roth M A, Malato J, VandenBerg S, Hann B, Atreya C E, Altschuler S J, Wu L F. A multi-modal data resource for investigating topographic heterogeneity in patient-derived xenograft tumors. Sci Data. 2019; 6(1):253. doi: 10.1038/s41597-019-0225-0. PubMed PMID: 31672976; PMCID: PMC6823477.
12. Corcoran R B, Ebi H, Turke A B, Coffee E M, Nishino M, Cogdill A P, Brown R D, Della Pelle P, Dias-Santagata D, Hung K E, Flaherty K T, Piris A, Wargo J A, Settleman J, Mino-Kenudson M, Engelman J A. EGFR-mediated re-activation of MAPK signaling contributes to insensitivity of BRAF mutant colorectal cancers to RAF inhibition with vemurafenib. Cancer Discov. 2012; 2(3):227-35. Epub 2012/03/27. doi: 10.1158/2159-8290.cd-11-0341. PubMed PMID: 22448344; PMCID: PMC3308191.
13. Prahallad A, Sun C, Huang S, Di Nicolantonio F, Salazar R, Zecchin D, Beijersbergen R L, Bardelli A, Bernards R. Unresponsiveness of colon cancer to BRAF(V600E) inhibition through feedback activation of EGFR. Nature. 2012; 483(7387):100-3. doi: 10.1038/nature10868. PubMed PMID: 22281684.
14. Yang H, Higgins B, Kolinsky K, Packman K, Bradley W D, Lee R J, Schostack K, Simcox M E, Kopetz S, Heimbrook D, Lestini B, Bollag G, Su F. Antitumor activity of BRAF inhibitor vemurafenib in preclinical models of BRAF-mutant colorectal cancer. Cancer Res. 2012; 72(3):779-89. Epub 2011/12/20. doi: 10.1158/0008-5472.can-11-2941. PubMed PMID: 22180495.
15. Manning G, Whyte D B, Martinez R, Hunter T, Sudarsanam S. The protein kinase complement of the human genome. Science. 2002; 298(5600):1912-34. Epub 2002/12/10. doi: 10.1126/science. 1075762298/5600/1912 [pii]. PubMed PMID: 12471243.
16. Erpel T, Courtneidge S A. Src family protein tyrosine kinases and cellular signal transduction pathways. Curr Opin Cell Biol. 1995; 7(2):176-82. Epub 1995/04/01. doi: 0955-0674(95)80025-5 [pii]10.1016/0955-0674(95)80025-5. PubMed PMID: 7612268.
17. Parsons S J, Parsons J T. Src family kinases, key regulators of signal transduction. Oncogene. 2004; 23(48):7906-9. Epub 2004/10/19. doi: 10.1038/sj.onc.1208160. PubMed PMID: 15489908.
18. Thomas S M, Brugge J S. Cellular functions regulated by Src family kinases. Annu Rev Cell Dev Biol. 1997; 13:513-609. Epub 1997/01/01. doi: 10.1146/annurev.cellbio.13.1.513. PubMed PMID: 9442882.
19. Pai R, Soreghan B, Szabo I L, Pavelka M, Baatar D, Tarnawski A S. Prostaglandin E2 transactivates EGF receptor: a novel mechanism for promoting colon cancer growth and gastrointestinal hypertrophy. Nat Med. 2002; 8(3):289-93. doi: 10.1038/nm0302-289. PubMed PMID: 11875501.
20. Buchanan F G, Gorden D L, Matta P, Shi Q, Matrisian L M, DuBois R N. Role of beta-arrestin 1 in the metastatic progression of colorectal cancer. Proc Natl Acad Sci USA. 2006; 103(5):1492-7. doi: 10.1073/pnas.0510562103. PubMed PMID: 16432186; PMCID: PMC1360588.
21. Castellone M D, Teramoto H, Williams B O, Druey K M, Gutkind J S. Prostaglandin E2 promotes colon cancer cell growth through a Gs-axin-beta-catenin signaling axis. Science. 2005; 310(5753):1504-10. doi: 10.1126/science.1116221. PubMed PMID: 16293724.
22. Dorsam R T, Gutkind J S. G-protein-coupled receptors and cancer. Nat Rev Cancer. 2007; 7(2):79-94. doi: 10.1038/nrc2069. PubMed PMID: 17251915.
23. Wang D, Dubois R N. Eicosanoids and cancer. Nat Rev Cancer. 2010; 10(3):181-93. doi: 10.1038/nrc2809. PubMed PMID: 20168319; PMCID: PMC2898136.
24. Wang D, Dubois R N. The role of COX-2 in intestinal inflammation and colorectal cancer. Oncogene. 2010; 29(6):781-8. Epub 2009/12/01. doi: 10.1038/onc.2009.421. PubMed PMID: 19946329; PMCID: PMC3181054.
25. Montero-Conde C, Ruiz-Llorente S, Dominguez J M, Knauf J A, Viale A, Sherman E J, Ryder M, Ghossein R A, Rosen N, Fagin J A. Relief of feedback inhibition of HER3 transcription by RAF and MEK inhibitors attenuates their antitumor effects in BRAF-mutant thyroid carcinomas. Cancer Discov. 2013; 3(5):520-33. Epub 2013/02/01. doi: 10.1158/2159-8290.cd-12-0531. PubMed PMID: 23365119; PMCID: PMC3651738.
26. Petricoin E F, Zoon K C, Kohn E C, Barrett J C, Liotta L A. Clinical proteomics: translating benchside promise into bedside reality. Nature reviews Drug discovery. 2002; 1(9):683-95. Epub 2002/09/05. doi: 10.1038/nrd891. PubMed PMID: 12209149.
27. Blume-Jensen P, Hunter T. Oncogenic kinase signalling. Nature. 2001; 411(6835):355-65. Epub 2001/05/18. doi: 10.1038/35077225. PubMed PMID: 11357143.
28. Fleuren E D, Zhang L, Wu J, Daly R J. The kinome ‘at large’ in cancer. Nat Rev Cancer. 2016; 16(2):83-98. doi: 10.1038/nrc.2015.18. PubMed PMID: 26822576.
29. Konieczkowski D J, Johannessen C M, Garraway L A. A Convergence-Based Framework for Cancer Drug Resistance. Cancer Cell. 2018; 33(5):801-15. Epub 2018/05/16. doi: 10.1016/j.ccell.2018.03.025. PubMed PMID: 29763622; PMCID: PMC5957297.
30. Roskoski R, Jr. Src protein-tyrosine kinase structure, mechanism, and small molecule inhibitors. Pharmacological research. 2015; 94:9-25. Epub 2015/02/11. doi: 10.1016/j.phrs.2015.01.003. PubMed PMID: 25662515.
31. Kopetz S. Targeting S R C and epidermal growth factor receptor in colorectal cancer: rationale and progress into the clinic. Gastrointest Cancer Res. 2007; 1(4 Suppl 2):S37-41. Epub 2007/07/01. PubMed PMID: 19360146; PMCID: 2666842.
32. Fabian J R, Daar I O, Morrison D K. Critical tyrosine residues regulate the enzymatic and biological activity of Raf-1 kinase. Mol Cell Biol. 1993; 13(11):7170-9. Epub 1993/11/01. doi: 10.1128/mcb.13.11.7170. PubMed PMID: 7692235; PMCID: PMC364778.
33. Marais R, Light Y, Paterson H F, Marshall C J. Ras recruits Raf-1 to the plasma membrane for activation by tyrosine phosphorylation. Embo j. 1995; 14(13):3136-45. Epub 1995/07/03. PubMed PMID: 7542586; PMCID: PMC394375.
34. Marais R, Light Y, Paterson H F, Mason C S, Marshall C J. Differential regulation of Raf-1, A-Raf, and B-Raf by oncogenic ras and tyrosine kinases. J Biol Chem. 1997; 272(7):4378-83. Epub 1997/02/14. doi: 10.1074/jbc.272.7.4378. PubMed PMID: 9020159.
35. Hu J, Stites E C, Yu H, Germino E A, Meharena H S, Stork P J S, Kornev A P, Taylor S S, Shaw A S. Allosteric activation of functionally asymmetric RAF kinase dimers. Cell. 2013; 154(5):1036-46. Epub 2013/09/03. doi: 10.1016/j.cell.2013.07.046. PubMed PMID: 23993095; PMCID: PMC3844432.
36. Chen R, Kim O, Yang J, Sato K, Eisenmann K M, McCarthy J, Chen H, Qiu Y. Regulation of Akt/PKB activation by tyrosine phosphorylation. J Biol Chem. 2001; 276(34):31858-62. Epub 2001/07/11. doi: 10.1074/jbc.C100271200 C100271200 [pii]. PubMed PMID: 11445557.
37. Coluccia A M, Benati D, Dekhil H, De Filippo A, Lan C, Gambacorti-Passerini C. SKI-606 decreases growth and motility of colorectal cancer cells by preventing pp60(c-Src)-dependent tyrosine phosphorylation of beta-catenin and its nuclear signaling. Cancer Res. 2006; 66(4):2279-86. Epub 2006/02/21. doi: 66/4/2279 [pii]10.1158/0008-5472.CAN-05-2057. PubMed PMID: 16489032.
38. Brembeck F H, Rosario M, Birchmeier W. Balancing cell adhesion and Wnt signaling, the key role of beta-catenin. Curr Opin Genet Dev. 2006; 16(1):51-9. Epub 2005/12/27. doi: S0959-437X(05)00223-6 [pii]10.1016/j.gde.2005.12.007. PubMed PMID: 16377174.
39. Silva C M. Role of STATs as downstream signal transducers in Src family kinase-mediated tumorigenesis. Oncogene. 2004; 23(48):8017-23. Epub 2004/10/19. doi: 1208159 [pii]10.1038/sj.onc.1208159. PubMed PMID: 15489919.
40. Comprehensive molecular characterization of human colon and rectal cancer. Nature. 2012; 487(7407):330-7. Epub 2012/07/20. doi: 10.1038/nature11252. PubMed PMID: 22810696; PMCID: PMC3401966.
Cancer. 2013; 13(1):11-26. Epub 2012/12/22. doi: nrc3419 [pii]10.1038/nrc3419. PubMed PMID: 23258168.
42. Kosumi K, Hamada T, Zhang S, Liu L, da Silva A, Koh H, Twombly T S, Mima K, Morikawa T, Song M, Nowak J A, Nishihara R, Saltz L B, Niedzwiecki D, Ou F S, Zemla T, Mayer R J, Baba H, Ng K, Giannakis M, Zhang X, Wu K, Giovannucci E L, Chan A T, Fuchs C S, Meyerhardt J A, Ogino S. Prognostic association of PTGS2 (COX-2) over-expression according to BRAF mutation status in colorectal cancer: Results from two prospective cohorts and CALGB 89803 (Alliance) trial. Eur J Cancer. 2019; 111:82-93. Epub 2019/03/04. doi: 10.1016/j.ejca.2019.01.022. PubMed PMID: 30826660; PMCID: PMC6436990.
43. Sharma M R, Wroblewski K, Polite B N, Knost J A, Wallace J A, Modi S, Sleckman B G, Taber D, Vokes E E, Stadler W M, Kindler H L. Dasatinib in previously treated metastatic colorectal cancer: a phase II trial of the University of Chicago Phase II Consortium. Investigational new drugs. 2012; 30(3):1211-5. Epub 2011/05/10. doi: 10.1007/s10637-011-9681-x. PubMed PMID: 21552992; PMCID: PMC4317401.
44. Parseghian C M, Parikh N U, Wu J Y, Jiang Z Q, Henderson L, Tian F, Pastor B, Ychou M, Raghav K, Dasari A, Fogelman D R, Katsiampoura A D, Menter D G, Wolff R A, Eng C, Overman M J, Thierry A R, Gallick G E, Kopetz S. Dual Inhibition of EGFR and c-Src by Cetuximab and Dasatinib Combined with FOLFOX Chemotherapy in Patients with Metastatic Colorectal Cancer. Clin Cancer Res. 2017; 23(15):4146-54. Epub 2017/03/11. doi: 10.1158/1078-0432.ccr-16-3138. PubMed PMID: 28280091; PMCID: PMC5540760.
45. Reddy S M, Kopetz S, Morris J, Parikh N, Qiao W, Overman M J, Fogelman D, Shureiqi I, Jacobs C, Malik Z, Jimenez C A, Wolff R A, Abbruzzese J L, Gallick G, Eng C. Phase II study of saracatinib (AZD0530) in patients with previously treated metastatic colorectal cancer. Investigational new drugs. 2015; 33(4):977-84. Epub 2015/06/13. doi: 10.1007/s10637-015-0257-z. PubMed PMID: 26062928.
46. Chen E Y, Blanke C D, Haller D G, Benson A B, Dragovich T, Lenz H J, Robles C, Li H, Mori M, Mattek N, Sanborn R E, Lopez C D. A Phase II Study of Celecoxib With Irinotecan, 5-Fluorouracil, and Leucovorin in Patients With Previously Untreated Advanced or Metastatic Colorectal Cancer. American journal of clinical oncology. 2018; 41(12):1193-8. Epub 2018/05/22. doi: 10.1097/coc.0000000000000465. PubMed PMID: 29782360; PMCID: PMC6240505.
47. André T, Tournigand C, Mineur L, Fellague-Chebra R, Flesch M, Mabro M, Hebbar M, Postel Vinay S, Bidard F C, Louvet C, de Gramont A. Phase II study of an optimized 5-fluorouracil-oxaliplatin strategy (OPTIMOX2) with celecoxib in metastatic colorectal cancer: a GERCOR study. Annals of oncology: official journal of the European Society for Medical Oncology. 2007; 18(1):77-81. Epub 2006/10/13. doi: 10.1093/annonc/md1336. PubMed PMID: 17030548.
48. Maiello E, Giuliani F, Gebbia V, Di Renzo N, Pezzella G, Romito S, Mallamaci R, Lopez M, Colucci G. FOLFIRI with or without celecoxib in advanced colorectal cancer: a randomized phase II study of the Gruppo Oncologico dell'Italia Meridionale (GOIM). Annals of oncology: official journal of the European Society for Medical Oncology. 2006; 17 Suppl 7:vii55-9. Epub 2006/06/09. doi: 10.1093/annonc/md1952. PubMed PMID: 16760295.
49. Sanjana N E, Shalem O, Zhang F. Improved vectors and genome-wide libraries for CRISPR screening. Nat Methods. 2014; 11(8):783-4. doi: 10.1038/nmeth.3047. PubMed PMID: 25075903; PMCID: PMC4486245.
50. Shalem O, Sanjana N E, Hartenian E, Shi X, Scott D A, Mikkelson T, Heckl D, Ebert B L, Root D E, Doench J G, Zhang F. Genome-scale CRISPR-Cas9 knockout screening in human cells. Science. 2014; 343(6166):84-7. doi: 10.1126/science. 1247005. PubMed PMID: 24336571; PMCID: PMC4089965.
51. Iglesias-Bartolome R, Torres D, Marone R, Feng X, Martin D, Simaan M, Chen M, Weinstein L S, Taylor S S, Molinolo A A, Gutkind J S. Inactivation of a Galpha(s)-PKA tumour suppressor pathway in skin stem cells initiates basal-cell carcinogenesis. Nat Cell Biol. 2015; 17(6):793-803. doi: 10.1038/ncb3164. PubMed PMID: 25961504; PMCID: PMC4449815.
52. Pan B, Olow A, Sun Q, Mori M, Lee P, Hartog M, Wang C, Wolf D, Yau C, van't Veer L, Coppe J P. Functional detection and inhibition of the targetable oncogenic kinome of chemotherapy-treated triple negative breast cancer cells. Cancer Research. 2016; 76(S4).
53. Mori M, Chen Z, Boudreau A, van't Veer L, Coppe J P. Kinase-sensing system to identify the oncogenic phospho-fingerprint of breast cancer. Journal of Clinical Oncology. 2013; 26(S).
54. Foucquier J, Guedj M. Analysis of drug combinations: current methodological landscape. Pharmacol Res Perspect. 2015; 3(3):e00149. doi: 10.1002/prp2.149. PubMed PMID: 26171228; PMCID: PMC4492765.
55. Berenbaum M C. What is synergy?Pharmacol Rev. 1989; 41(2):93-141. PubMed PMID: 2692037.
56. Y Xi, Y Wei, B Sennino, A Ulsamer, I Kwan, A N Brumwell, K Tan, M K Aghi, D M McDonald, D M Jablons, H A Chapman. Identification of pY654-β-catenin as a critical co-factor in hypoxia-inducible factor-1α signaling and tumor responses to hypoxia. Oncogene. 2013 Oct. 17; 32(42):5048-57. doi: 10.1038/onc.2012.530. Epub 2012 Dec. 17. PMID: 23246962 PMCID: PMC3871884 DOI: 10.1038/onc.2012.530
57. Andreas Herbst, Vindi Jurinovic, Stefan Krebs, Susanne E Thieme, Helmut Blum, Burkhard Göke, Frank T Kolligs. Comprehensive analysis of β-catenin target genes in colorectal carcinoma cell lines with deregulated Wnt/μ-catenin signaling. BMC Genomics. 2014 Jan. 28; 15:74. doi: 10.1186/1471-2164-15-74. PMID: 24467841 PMCID: PMC3909937 DOI: 10.1186/1471-2164-15-74
58. Daniel L Simmons, Regina M Botting, Timothy Hla. Cyclooxygenase isozymes: the biology of prostaglandin synthesis and inhibition. Pharmacol Rev. 2004 September; 56(3):387-437. doi: 10.1124/pr.56.3.3. PMID: 15317910 DOI: 10.1124/pr.56.3.3
59. Girotti M R, Lopes F, Preece N, Niculescu-Duvaz D, Zambon A, Davies L, Whittaker S, Saturno G, Viros A, Pedersen M, Suijkerbuijk B M, Menard D, McLeary R, Johnson L, Fish L, Ejiama S, Sanchez-Laorden B, Hohloch J, Carragher N, Macleod K, Ashton G, Marusiak A A, Fusi A, Brognard J, Frame M, Lorigan P, Marais R, Springer C. Paradox-breaking RAF inhibitors that also target SRC are effective in drug-resistant BRAF mutant melanoma. Cancer Cell. 2015 Jan. 12; 27(1):85-96. doi: 10.1016/j.ccell.2014.11.006. Epub 2014 Dec. 11. PMID: 25500121
60. Girotti M R, Pedersen M, Sanchez-Laorden B, Viros A, Turajlic S, Niculescu-Duvaz D, Zambon A, Sinclair J, Hayes A, Gore M, Lorigan P, Springer C, Larkin J, Jorgensen C, Marais R. Inhibiting EGF receptor or SRC family kinase signaling overcomes BRAF inhibitor resistance in melanoma. Cancer Discov. 2013 February; 3(2):158-67. doi: 10.1158/2159-8290.CD-12-0386. Epub 2012 Dec. 14. PMID: 23242808

1. A method of treating a serine/threonine-protein kinase B-Raf (BRAF) intrinsically resistant cancer in a subject, comprising administering to the subject a combined effective amount of a BRAF axis inhibitor and a cyclooxygenase-2 (COX-2) inhibitor.

2. The method of claim 1, wherein the BRAF axis inhibitor comprises a BRAF inhibitor, a MEK inhibitor, an ERK inhibitor, or a combination thereof.

3. The method of claim 2, wherein the BRAF axis inhibitor comprises a BRAF inhibitor and an MEK inhibitor.

4. (canceled)

5. (canceled)

6. The method of claim 2, wherein the MEK inhibitor is trametinib, binimetinib, cobimetinib, selumetinib, or a combination thereof.

7. The method of claim 6, wherein the MEK inhibitor is trametinib.

8. The method of claim 2, wherein the ERK inhibitor is ulixertinib (BVD-523), ravoxertinib (GDC-0994), LY3214996, LTT462, or a combination thereof.

9. The method of claim 2, wherein the BRAF inhibitor is encorafenib, vemurafenib, dabrafenib, or a combination thereof.

10. The method of claim 9, wherein the BRAF inhibitor is vemurafenib or encorafenib.

11. The method of claim 1, wherein the COX-2 inhibitor is celecoxib, valdecoxib, ketorolac, rofecoxib, acetylsalicylic acid, choline magnesium trisalicylate, diflunisal, salsalate, fenoprofen, flurbiprofen, ibuprofen, ketoprofen, naproxen, oxaprozin, diclofenac, indomethacin, sulindac, tolmetin, meloxicam, piroxicam, meclofenamate, nefenamic acid, nabumetone, etodalac, or a combination thereof.

12. The method of claim 11, wherein the COX-2 inhibitor is celecoxib.

13. (canceled)

14. (canceled)

15. The method of claim 1, further comprising administering to the subject an epidermal growth factor receptor (EGFR) inhibitor.

16. The method of claim 15, wherein the EGFR inhibitor is panitumumab, cetuximab, gefitinib, erlotinib, neratinib, lapatinib, necitumumab, or a combination thereof.

17. The method of claim 1, further comprising administering to the subject a SRC inhibitor.

18. The method of claim 17, wherein the SRC inhibitor is dasatinib, saracatinib, bosutinib, ponatinib, imatinib, or a combination thereof.

19. The method of claim 1, wherein the BRAF intrinsically resistant cancer is colorectal cancer, thyroid cancer, glioblastoma, sarcoma, or hepto-sarcoma.

20. The method of claim 1, wherein the BRAF intrinsically resistant cancer is colorectal cancer.

21. A pharmaceutical kit comprising:
(i) a serine/threonine-protein kinase B-Raf (BRAF) axis inhibitor; and
(ii) a and a cyclooxygenase-2 (COX-2) inhibitor.

22. (canceled)

23. The pharmaceutical kit of claim 21, wherein the BRAF axis inhibitor comprises a BRAF inhibitor and a MEK inhibitor.

24. (canceled)

25. (canceled)

26. (canceled)

27. (canceled)

28. (canceled)

29. (canceled)

30. (canceled)

31. (canceled)

32. (canceled)

33. The pharmaceutical kit of claim 21, further comprising an epidermal growth factor receptor (EGFR) inhibitor.

34. (canceled)

35. The pharmaceutical kit of claim 21, further comprising a SRC inhibitor.

36. (canceled)

37. (canceled)

38. (canceled)

39. (canceled)

40. (canceled)


SEQUENCE LISTING (29 sequences)

SEQ ID NO: 1 - DNA, 21 bp, synthetic construct
  source 1..21: mol_type = other DNA
gctgacagtttgtggcatctt

SEQ ID NO: 2 - DNA, 21 bp, synthetic construct
  source 1..21: mol_type = other DNA
catcctcaggaaccaacaatt

SEQ ID NO: 3 - DNA, 25 bp, synthetic construct
  source 1..25: mol_type = other DNA
caccgctacaacatggtcatccggg

SEQ ID NO: 4 - DNA, 25 bp, synthetic construct
  source 1..25: mol_type = other DNA
aaaccccggatgaccatgttgtagc

SEQ ID NO: 5 - DNA, 25 bp, synthetic construct
  source 1..25: mol_type = other DNA
aaaccccggatgaccatgttgtagc

SEQ ID NO: 6 - DNA, 23 bp, synthetic construct
  source 1..23: mol_type = other DNA
gcgaaggcacaatcatcaatgtt

SEQ ID NO: 7 - DNA, 21 bp, synthetic construct
  source 1..21: mol_type = other DNA
gtcaagaggcgaacacacaac

SEQ ID NO: 8 - DNA, 21 bp, synthetic construct
  source 1..21: mol_type = other DNA
ttggacggacaggatgtatgc

SEQ ID NO: 9 - DNA, 22 bp, synthetic construct
  source 1..22: mol_type = other DNA
aacttgagctgctggagggaca

SEQ ID NO: 10 - DNA, 22 bp, synthetic construct
  source 1..22: mol_type = other DNA
tcttggccagcatggaaaactc

SEQ ID NO: 11 - DNA, 19 bp, synthetic construct
  source 1..19: mol_type = other DNA
gggagggtgacaagcacac

SEQ ID NO: 12 - DNA, 20 bp, synthetic construct
  source 1..20: mol_type = other DNA
agcttcgagccaatggtgag

SEQ ID NO: 13 - DNA, 21 bp, synthetic construct
  source 1..21: mol_type = other DNA
aatgctgctgaactgaataga

SEQ ID NO: 14 - DNA, 20 bp, synthetic construct
  source 1..20: mol_type = other DNA
aaccggtcctagcgaaaacc

SEQ ID NO: 15 - DNA, 19 bp, synthetic construct
  source 1..19: mol_type = other DNA
caacaccaggcggaacgaa

SEQ ID NO: 16 - DNA, 23 bp, synthetic construct
  source 1..23: mol_type = other DNA
gcccaataaggagtgtaaggact

SEQ ID NO: 17 - DNA, 19 bp, synthetic construct
  source 1..19: mol_type = other DNA
agaacaccccgatgacgga

SEQ ID NO: 18 - DNA, 22 bp, synthetic construct
  source 1..22: mol_type = other DNA
ggcatcattatgtacccggaat

SEQ ID NO: 19 - DNA, 21 bp, synthetic construct
  source 1..21: mol_type = other DNA
catgtacgttgctatccaggc

SEQ ID NO: 20 - DNA, 21 bp, synthetic construct
  source 1..21: mol_type = other DNA
ctccttaatgtcacgcacgat

SEQ ID NO: 21 - DNA, 19 bp, synthetic construct
  source 1..19: mol_type = other DNA
atttgggtcgcggttcttg

SEQ ID NO: 22 - DNA, 21 bp, synthetic construct
  source 1..21: mol_type = other DNA
tgccttgacattctcgatggt

SEQ ID NO: 23 - protein, 766 aa, Homo sapiens [sequence omitted]

SEQ ID NO: 24 - protein, 119 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 25 - protein, 107 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 26 - protein, 119 aa, synthetic construct [sequence omitted]

SEQ ID NO: 27 - protein, 107 aa, synthetic construct [sequence omitted]

SEQ ID NO: 28 - protein, 121 aa, synthetic construct [sequence omitted]

SEQ ID NO: 29 - protein, 107 aa, synthetic construct [sequence omitted]